SEQUENCE LISTING (48 sequences)

SEQ ID NO: 1 - protein, 21 aa, synthetic construct
  SITE 8: note = phosphorylation
  SITE 11: note = phosphorylation
  SITE 14: note = phosphorylation
GDRSGYSSPGSPGTPGSRSRT

SEQ ID NO: 2 - protein, 16 aa, synthetic construct
  SITE 4: note = phosphorylation
  SITE 12: note = phosphorylation
VYKSPVVSGDTSPRHL

SEQ ID NO: 3 - protein, 18 aa, synthetic construct
  SITE 11: note = phosphorylation
  SITE 16: note = phosphorylation
SSTGSIDMVDSPQLATLA

SEQ ID NO: 4 - protein, 16 aa, synthetic construct
  REGION 1..16: note = tau peptide
VYKSPVVSGDTSPRHL

SEQ ID NO: 5 - protein, 30 aa, synthetic construct
  SITE 18: note = phosphorylation
  SITE 26: note = phosphorylation
RENAKAKTDHGAEIVYKSPVVSGDTSPRHL

SEQ ID NO: 6 - protein, 16 aa, synthetic construct
  SITE 14: note = phosphorylation
RQEFEVMEDHAGTYGL

SEQ ID NO: 7 - protein, 16 aa, synthetic construct
  SITE 7: note = phosphorylation
  SITE 9: note = phosphorylation
PGSRSRTPSLPTPPTR

SEQ ID NO: 8 - protein, 16 aa, synthetic construct
  SITE 7: note = phosphorylation
  SITE 10: note = phosphorylation
GYSSPGSPGTPGSRSR

SEQ ID NO: 9 - protein, 18 aa, synthetic construct
  SITE 4: note = phosphorylation
  SITE 9: note = phosphorylation
GDTSPRHLSNVSSTGSID

SEQ ID NO: 10 - protein, 17 aa, synthetic construct
  SITE 3: note = phosphorylation
  SITE 6: note = phosphorylation
  SITE 13: note = phosphorylation
  SITE 15: note = phosphorylation
PGSPGTPGSRSRTPSLP

SEQ ID NO: 11 - protein, 12 aa, synthetic construct
  SITE 3: note = phosphorylation
HLSNVSSTGSID

SEQ ID NO: 12 - protein, 10 aa, synthetic construct
  SITE 6: note = phosphorylation
VSGDTSPRHL

SEQ ID NO: 13 - protein, 55 aa, synthetic construct
  MOD_RES 55: note = C-terminal amide
AKFVAAWTLKAAAVVRQYIKANSKFIGITELVVRFNNFTVSFWLRVPKVSASHLE

SEQ ID NO: 14 - protein, 55 aa, synthetic construct
  SITE 54: note = palmitoylated lysine
  SITE 55: note = palmitoylated lysine
  MOD_RES 55: note = C-terminal amide
AKFVAAWTLKAAAGSQYIKANSKFIGITELGSFNNFTVSFWLRVPKVSASHLEKK

SEQ ID NO: 15 - protein, 75 aa, synthetic construct
  MOD_RES 75: note = C-terminal amide
AKFVAAWTLKAAAGSQYIKANSKFIGITELGSFNNFTVSFWLRVPKVSASHLEGSLINSTKIYSYFPSVISKVNQ

SEQ ID NO: 16 - protein, 53 aa, synthetic construct
  MOD_RES 53: note = C-terminal amide
AKFVAAWTLKAAARRQYIKANSKFIGITELRRFNNFTVSFWLRVPKVSASHLE

SEQ ID NO: 17 - protein, 53 aa, synthetic construct
  MOD_RES 53: note = C-terminal amide
AKFVAAWTLKAAARKQYIKANSKFIGITELRKFNNFTVSFWLRVPKVSASHLE

SEQ ID NO: 18 - DNA, 24 bp, synthetic construct
  modified_base 1..24: mod_base = OTHER; note = phosphorothioate (ps) internucleotide linkages
  source 1..24: mol_type = other DNA
tcgtcgttttgtcgttttgtcgtt

SEQ ID NO: 19 - DNA, 22 bp, synthetic construct
  modified_base 1..22: mod_base = OTHER; note = phosphorothioate (ps) internucleotide linkages
  source 1..22: mol_type = other DNA
tgactgtgaacgttcgagatga

SEQ ID NO: 20 - DNA, 22 bp, synthetic construct
  modified_base 1..22: mod_base = OTHER; note = phosphorothioate (ps) internucleotide linkages
  source 1..22: mol_type = other DNA
tcgtcgttttcggcgcgcgccg

SEQ ID NO: 21 - DNA, 20 bp, synthetic construct
  modified_base 1..3: mod_base = OTHER; note = phosphorothioate (ps) internucleotide linkages
  modified_base 3..14: mod_base = OTHER; note = phosphodiester (po) internucleotide linkages
  modified_base 14..20: mod_base = OTHER; note = phosphorothioate (ps) internucleotide linkages
  source 1..20: mol_type = other DNA
gggggacgatcgtcgggggg

SEQ ID NO: 22 - DNA, 21 bp, synthetic construct
  modified_base 1..4: mod_base = OTHER; note = phosphorothioate (ps) internucleotide linkages
  modified_base 4..15: mod_base = OTHER; note = phosphorothioate (ps) internucleotide linkages
  modified_base 15..21: mod_base = OTHER; note = phosphorothioate (ps) internucleotide linkages
  source 1..21: mol_type = other DNA
ggggacgacgtcgtggggggg

SEQ ID NO: 23 - protein, 13 aa, synthetic construct
AKFVAAWTLKAAA

SEQ ID NO: 24 - protein, 15 aa, synthetic construct
  REGION 1..15: note = P2
QYIKANSKFIGITEL

SEQ ID NO: 25 - protein, 21 aa, synthetic construct
FNNFTVSFWLRVPKVSASHLE

SEQ ID NO: 26 - protein, 20 aa, synthetic construct
LINSTKIYSYFPSVISKVNQ

SEQ ID NO: 27 - protein, 25 aa, synthetic construct
  LIPID 1: note = palmitoylated
  SITE 2: note = palmitoylated
  SITE 10: note = phosphorylation
  SITE 13: note = phosphorylation
  SITE 16: note = phosphorylation
  LIPID 24: note = palmitoylated
  LIPID 25: note = palmitoylated
KKGDRSGYSSPGSPGTPGSRSRTKK

SEQ ID NO: 28 - protein, 20 aa, synthetic construct
  LIPID 1: note = palmitoylated
  LIPID 2: note = palmitoylated
  SITE 6: note = phosphorylation
  SITE 14: note = phosphorylation
  LIPID 19: note = palmitoylated
  LIPID 20: note = palmitoylated
KKVYKSPVVSGDTSPRHLKK

SEQ ID NO: 29 - protein, 22 aa, synthetic construct
  LIPID 1: note = palmitoylated
  LIPID 2: note = palmitoylated
  SITE 13: note = phosphorylation
  SITE 18: note = phosphorylation
  LIPID 21: note = palmitoylated
  LIPID 22: note = palmitoylated
KKSSTGSIDMVDSPQLATLAKK

SEQ ID NO: 30 - protein, 20 aa, synthetic construct
  LIPID 1: note = palmitoylated
  LIPID 2: note = palmitoylated
  LIPID 19: note = palmitoylated
  LIPID 20: note = palmitoylated
KKVYKSPVVSGDTSPRHLKK

SEQ ID NO: 31 - protein, 34 aa, synthetic construct
  LIPID 1: note = palmitoylated
  LIPID 2: note = palmitoylated
  SITE 20: note = phosphorylation
  SITE 28: note = phosphorylation
  LIPID 33: note = palmitoylated
  LIPID 34: note = palmitoylated
KKRENAKAKTDHGAEIVYKSPVVSGDTSPRHLKK

SEQ ID NO: 32 - protein, 20 aa, synthetic construct
  LIPID 1: note = palmitoylated
  LIPID 2: note = palmitoylated
  SITE 16: note = phosphorylation
  LIPID 19: note = palmitoylated
  LIPID 20: note = palmitoylated
KKRQEFEVMEDHAGTYGLKK

SEQ ID NO: 33 - protein, 20 aa, synthetic construct
  LIPID 1: note = palmitoylated
  LIPID 2: note = palmitoylated
  SITE 9: note = phosphorylation
  SITE 11: note = phosphorylation
  LIPID 19: note = palmitoylated
  LIPID 20: note = palmitoylated
KKPGSRSRTPSLPTPPTRKK

SEQ ID NO: 34 - protein, 20 aa, synthetic construct
  LIPID 1: note = palmitoylated
  LIPID 2: note = palmitoylated
  SITE 9: note = phosphorylation
  SITE 12: note = phosphorylation
  LIPID 19: note = palmitoylated
  LIPID 20: note = palmitoylated
KKGYSSPGSPGTPGSRSRKK

SEQ ID NO: 35 - protein, 22 aa, synthetic construct
  LIPID 1: note = palmitoylated
  LIPID 2: note = palmitoylated
  SITE 6: note = phosphorylation
  SITE 11: note = phosphorylation
  LIPID 21: note = palmitoylated
  LIPID 22: note = palmitoylated
KKGDTSPRHLSNVSSTGSIDKK

SEQ ID NO: 36 - protein, 21 aa, synthetic construct
  LIPID 1: note = palmitoylated
  LIPID 2: note = palmitoylated
  SITE 5: note = phosphorylation
  SITE 8: note = phosphorylation
  SITE 15: note = phosphorylation
  SITE 17: note = phosphorylation
  LIPID 20: note = palmitoylated
  LIPID 21: note = palmitoylated
KKPGSPGTPGSRSRTPSLPKK

SEQ ID NO: 37 - protein, 16 aa, synthetic construct
  LIPID 1: note = palmitoylated
  LIPID 2: note = palmitoylated
  SITE 5: note = phosphorylation
  LIPID 15: note = palmitoylated
  LIPID 16: note = palmitoylated
KKHLSNVSSTGSIDKK

SEQ ID NO: 38 - protein, 14 aa, synthetic construct
  LIPID 1: note = palmitoylated
  LIPID 2: note = palmitoylated
  SITE 8: note = phosphorylation
  LIPID 13: note = palmitoylated
  LIPID 14: note = palmitoylated
KKVSGDTSPRHLKK

SEQ ID NO: 39 - protein, 55 aa, synthetic construct; identical to SEQ ID NO: 13

SEQ ID NO: 40 - protein, 53 aa, synthetic construct
AKFVAAWTLKAAAGSQYIKANSKFIGITELGSFNNFTVSFWLRVPKVSASHLE

SEQ ID NO: 41 - protein, 75 aa, synthetic construct; identical to SEQ ID NO: 15

SEQ ID NO: 42 - protein, 53 aa, synthetic construct; identical to SEQ ID NO: 16

SEQ ID NO: 43 - protein, 53 aa, synthetic construct; identical to SEQ ID NO: 17

SEQ ID NO: 44 - protein, 57 aa, synthetic construct
  LIPID 56: note = palmitoylated
  LIPID 57: note = palmitoylated
  MOD_RES 57: note = C-terminal amide
AKFVAAWTLKAAAVVRQYIKANSKFIGITELVVRFNNFTVSFWLRVPKVSASHLEKK

SEQ ID NO: 45 - protein, 17 aa, synthetic construct
  SITE 5: note = phosphorylation
  SITE 13: note = phosphorylation
  REGION 1: note = Biotin-LC linker (Ahx)
GVYKSPVVSGDTSPRHL

SEQ ID NO: 46 - protein, 17 aa, synthetic construct
  REGION 1: note = Biotin-LC linker (Ahx)
GVYKSPVVSGDTSPRHL

SEQ ID NO: 47 - protein, 17 aa, synthetic construct
  REGION 1: note = Biotin-LC linker (Ahx)
  SITE 4: note = phosphorylation
  SITE 12: note = phosphorylation
VYKSPVVSGDTSPRHLS

SEQ ID NO: 48 - protein, 17 aa, synthetic construct
  REGION 1: note = Biotin-LC linker (Ahx)
VYKSPVVSGDTSPRHLS

(19) United States
(12) Patent Application Publication
(10) Pub. No.: US 2025/0144212 A1
(43) Pub. Date: May 8, 2025

(54) LIPOSOMES CONTAINING PHOSPHORYLATED TAU PEPTIDES FOR INDUCING SUSTAINED IMMUNE RESPONSES

(71) Applicants: Janssen Pharmaceuticals, Inc., Titusville, NJ (US); AC Immune SA, Lausanne (CH)

(72) Inventors: Andrea Pfeifer, St-Légier (CH); Andreas Muhs, Cugy (CH); Wendy Galpern, Boston, MA (US); Clara Theunis, Beerse (BE); Lennert Steukers, Pulle (BE)

(21) Appl. No.: 19/010,408

(22) Filed: Jan. 6, 2025

Related U.S. Application Data
(63) Continuation of application No. 18/343,349, filed on Jun. 28, 2023.
Application No. 18/343,349 is a continuation of application No. PCT/US2022/074902, filed on Aug. 12, 2022.
(60) Provisional application No. 63/260,227, filed on Aug. 12, 2021.
(60) Provisional application No. 63/263,541, filed on Nov. 4, 2021.
(60) Provisional application No. 63/267,975, filed on Feb. 14, 2022.

(51) Int. Cl. A61K 40/30; A61K 9/1271; A61K 39/00; A61K 40/11

(52) U.S. Cl. CPC A61K 40/30; A61K 9/1271; A61K 39/00; A61K 40/11; A61K 2039/54; A61K 2039/545; A61K 2039/55555; A61K 2039/55561; A61K 2039/55572; A61K 2039/57

(57) ABSTRACT

Methods for inducing a sustained immune response against phosphorylated Tau in humans are described. The methods include administering to the subject an effective amount of liposomes including a toll-like receptor 4 agonist, a helper T-cell epitope, a lipidated CpG oligonucleotide, and a Tau phosphopeptide presented on the surface of the liposome to thereby obtain the sustained immune response.

### CROSS-REFERENCE TO RELATED APPLICATION

This application is a continuation of U.S. patent application Ser. No. 18/343,349, filed Jun. 28, 2023, which is a continuation of International Application No. PCT/US2022/074902, filed on Aug. 12, 2022 and published as WO 2023/019241 on Feb. 16, 2023, which claims priority to each of U.S. Provisional Patent Application No. 63/260,227, filed Aug. 12, 2021, U.S. Provisional Patent Application No. 63/263,541, filed Nov. 4, 2021, and U.S. Provisional Patent Application No. 63/267,975, filed Feb. 14, 2022, the disclosure of each of which foregoing applications is incorporated herein by reference in its entirety.

### REFERENCE TO AN ELECTRONIC SEQUENCE LISTING

This application contains a sequence listing, which is submitted electronically. The contents of the electronic sequence listing (065794.7US6.xml; Size: 94,208 bytes; and Date of Creation: Jan. 6, 2025) is herein incorporated by reference in its entirety.

### FIELD OF THE INVENTION

The present invention is in the field of medicine. The invention in particular relates to a liposome that contains a phosphorylated Tau peptide for inducing a sustained antibody response against phosphorylated Tau protein (pTau) in a subject in need of preventing or treating Tauopathy, such as Alzheimer's Disease.

### BACKGROUND

Alzheimer's Disease (AD) is a progressive debilitating neurodegenerative disease that affects an estimated 44 million people worldwide (Alzheimers.net). AD therapies that are currently commercialized aim to act on the clinical symptoms, but do not target the pathogenic processes that underlie the disease (disease-modifying effect). Unfortunately, the current therapies are only minimally efficacious, and there is therefore an urgent need to develop and test additional preventive and therapeutic measures.

The hallmark pathologies for Alzheimer's Disease are an accumulation of extracellular plaques comprising notably aggregated amyloid beta protein and intracellular “tangles” or aggregations of hyperphosphorylated Tau protein. The molecular events that lead to accumulation of these proteins are poorly characterized. For amyloid, it is hypothesized that aberrant cleavage of the amyloid precursor protein leads to an accumulation of the aggregation-prone fragment comprising amino acids 1-42. For Tau, it is hypothesized that dysregulation of either kinases, phosphatases, or both, leads to aberrant phosphorylation of Tau. Once Tau becomes hyperphosphorylated it loses the ability to effectively bind and stabilize microtubules, and instead accumulates in the cytoplasm of the affected neuron. The unbound and hyperphosphorylated Tau appears to form first oligomers and then higher order aggregates, the presence of which presumably negatively affects the function of the neuron in which they form, perhaps via interruption of normal axonal transport.

In developed nations, individuals diagnosed with Alzheimer's Disease or other dementing Tauopathies are commonly treated with cholinesterase inhibitors (e.g., Aricept®) or memantine (e.g., Namenda™). These drugs, although reasonably well tolerated, have very modest efficacy. For example, Aricept® delays the worsening of symptoms for 6-12 months in approximately 50% of the treated individuals. The remainder of treatment is non-pharmacologic, and focuses on making patients more capable of managing day to day tasks as their cognitive ability declines.

The results of ADAMANT, a 24-month double-blinded, parallel-arm, randomized Phase 2 multicenter placebo-controlled trial of AADvac1, an active peptide vaccine designed to target pathological Tau in Alzheimer's Disease (EudraCT 2015-000630-30) has been recently published (Novak et al., Nature Aging vol 1: 521-534, 2021). The AADvac1 contains a synthetic peptide derived from amino acids 294 to 305 of the Tau sequence coupled to keyhole limpet hemocyanin (KLH) through an N-terminal cysteine. Eleven doses of AADvac1 were administered to patients with mild AD dementia at 40 μg per dose over the course of the trial. Although the vaccine induced high levels of IgG antibodies, no significant effects were found in cognitive and functional tests on the whole study sample (Id.).

ACI-35, a vaccine using a synthetic peptide based on human p-Tau396/404 was shown to have improved motor abilities and extended survival of mice carrying a P301L mutation (Theunis et al., PLOS ONE. 2013. 8(8): e72301). In a Phase 1b study, ACI-35 was well-tolerated and elicited an antibody response, with a limited boostability.

There is a need for a safe and effective treatment for neuronal degenerative disease, such as Alzheimer's Disease.

### SUMMARY OF THE INVENTION

The invention is based on findings from clinical studies of an improved liposomal vaccine comprising a phosphorylated Tau peptide presented on the surface of the liposome. The vaccine induced potent and sustained immune response, such as sustained antibody responses against pTau and the antibody responses could be boosted by booster shots.

Accordingly, in one general aspect, the invention provides a method of inducing an antibody response against a phosphorylated Tau protein (pTau) in a human subject in need thereof, comprising administering to the subject an effective amount of a liposome containing:
(1) a Tau phosphopeptide consisting of an amino acid sequence selected from the group consisting of SEQ ID NO:27 to SEQ ID NO:29 and SEQ ID NO:31 to SEQ ID NO:38 at an amount of 25-750 nmoles, such as 300 μg to 1800 μg, per dose;
(2) a toll-like receptor 4 agonist comprising monophosphoryl lipid A;
(3) a helper T-cell epitope having an amino acid sequence selected from the group consisting of SEQ ID NO:13 to SEQ ID NO:17, SEQ ID NO:23 to SEQ ID NO:26, and SEQ ID NO: 39 to SEQ ID NO:44; and
(4) a CpG oligonucleotide having a nucleotide sequence selected from the group consisting of SEQ ID NO:18 to SEQ ID NO:22,
   wherein:
the Tau phosphopeptide is presented on the surface of the liposome, and
the antibody response lasts at least 6 weeks, such as at least 6, 7, 8, 9, 10 weeks after the initial administration of the effective amount of the liposome to the human subject.

In some embodiments, the effective amount of the liposome comprises:
(1) the Tau phosphopeptide consisting of the amino acid sequence of SEQ ID NO: 28 at the amount of 300 μg to 1800 μg per dose;
(2) the toll-like receptor 4 agonist at an amount of 100 μg to 585 μg per dose;
(3) the helper T-cell epitope at an amount of 75 μg to 550 μg per dose; and
(4) the CpG oligonucleotide at an amount of 100 μg to 1000 μg per dose.

In some embodiment, the CpG oligonucleotide has one or more phosphorothioate internucleotide linkages, and the CpG oligonucleotide is covalently linked to at least one lipophilic group, optionally via a PEG linker.

In some embodiments, the Tau phosphopeptide is administered at an amount of about 25 nmoles to about 750 nmoles per dose, such as about 29.7 nmoles to about 742.5 nmoles per dose, preferably about 90 nmoles to about 715 nmoles, such as about 89.1 nmoles to about 712.8 nmoles per dose, or about 90 nmoles to about 535 nmoles per dose, such as about 89.1 nmoles to about 534.6 nmoles per dose, or about 90 nmoles to about 275 nmoles per dose, such as about 89.1 nmoles to about 267.3 nmoles per dose. In certain embodiments, the Tau phosphopeptide consists of an amino acid sequence selected from the group consisting of SEQ ID NO:27 to SEQ ID NO: 29 and SEQ ID NO:31 to SEQ ID NO:38, preferably consists of an amino acid sequence of SEQ ID NO:28. In one embodiment, the tetrapalmitoylated Tau phosphopeptide is administered at an amount of 100 μg to 2500 μg per dose, corresponding to 29.7 nmoles to 742.5 nmoles per dose, preferably 300 μg to 2400 μg per dose, corresponding to 89.1 nmoles to 712.8 nmols per dose, such as 300 μg, 900 μg, 1800 μg or 2400 μg per dose, corresponding to 89.1 nmoles, 267.3 nmoles, 534.6 nmoles or 712.8 nmoles per dose.

In certain embodiments, the effective amount of liposomes comprises the toll-like receptor 4 agonist at an amount of 30 μg to 900 μg, preferably 100 μg to 585 μg, per dose. In certain embodiments, the effective amount of liposomes comprises the toll-like receptor agonist monophosphoryl hexa-acyl Lipid A, 3-deacyl at an amount of 30 μg to 900 μg, preferably 100 μg to 585 μg, per dose.

In certain embodiments, the effective amount of liposomes comprises the helper T-cell epitope at an amount of 25 μg to 625 μg, preferably 75 μg to 550 μg, such as 75 μg to 450 μg, per dose. In certain embodiments, the effective amount of liposomes comprises a T50 helper T-cell epitope consisting of the amino acid sequence of SEQ ID NO: 13 at an amount of 25 μg to 625 μg, preferably 75μg to 450 μg, per dose. In certain embodiments, the effective amount of liposomes comprises the helper T-cell epitope at an amount of about 2 nmoles to about 110 nmoles per dose, such as about 4.02 nmoles to about 100.44 nmoles per dose, or about 4 nmoles to about 75 nmoles per dose, such as about 4.02 nmoles to about 72.32 nmoles per dose, or about 10 nmoles to about 105 nmoles per dose, such as about 12.06 nmoles to about 100.44 nmoles per dose, or about 70 to about 105 nmoles per dose, such as about 72.32 nmoles to about 100.44 nmoles per dose. In certain embodiments, the effective amount of liposomes comprises a T50 helper T-cell epitope consisting of the amino acid sequence of SEQ ID NO: 13 at an amount of about 3 nmoles to about 105 nmoles per dose, preferably about 10 nmoles to about 105 nmoles per dose, such as about 12.06 nmoles to about 100.44 nmoles per dose. In one embodiment, the effective amount of liposomes comprises the helper T-cell epitope at an amount of 2 to 5 nmoles per dose, e.g., 2, 3, 4 or 5 nmoles per dose or any value in between, such as about 3.82, 3.92, 4.02 or 4.12 nmoles per dose. In another embodiment, the effective amount of liposomes comprises the helper T-cell epitope at an amount of 10 to 15 nmoles per dose, such as 10, 11, 12, 13, 14 or 15 nmoles per dose, or any value in between, such as 11.86, 11.96, 12.06, 12.16 nmoles per dose. In another embodiment, the effective amount of liposomes comprises the helper T-cell epitope at an amount of 70 to 75 nmoles per dose, such as 70, 71, 72, 73, 74 or 75 nmoles per dose, or any value in between, such as 72.02, 72.12, 72.22, 72.32, 72.42 nmoles per dose. In yet another embodiment, the effective amount of liposomes comprises the helper T-cell epitope at an amount of 98 to 103 nmoles per dose, such as 98, 99, 100, 101, 102 or 103 nmoles per dose, or any value in between, such as 100.24, 100.34, 100.44, 100.54 or 100.64 nmoles per dose.

In certain embodiments, the effective amount of liposomes comprises the lipidated CpG oligonucleotide at an amount of 50 μg to 1250 μg, preferably 100 μg to 1000 μg, such as 150 μg to 800 μg, per dose. In certain embodiments, the effective amount of liposomes comprises a CpG oligonucleotide consisting of the nucleotide sequence of SEQ ID NO:18 at an amount of 50 μg to 1250 μg, preferably 150 μg to 800 μg, per dose.

In certain embodiments, the liposomes are administered subcutaneously.

In certain embodiments, the liposomes are administered intramuscularly.

In certain embodiments, the liposome further comprises one or more lipids selected from the group consisting of 1,2-dimyristoyl-sn-glycero-3-phosphocholine (DMPC), 1,2-dimyristoyl-sn-glycero-3-phosphoryl-3′-rac-glycerol (DMPG), and cholesterol.

In certain embodiments, the liposome comprises:
(1) the Tau phosphopeptide having the amino acid sequence of SEQ ID NO:28;
(2) the toll-like receptor 4 agonist comprising monophosphoryl hexa-acyl Lipid A, 3-deacyl;
(3) the helper T-cell epitope comprising the amino acid sequence of SEQ ID NO: 39;
(4) the lipidated CpG oligonucleotide comprising the nucleotide sequence of SEQ ID NO: 18; and
(5) at least one lipid selected from the group consisting of 1,2-dimyristoyl-sn-glycero-3-phosphocholine (DMPC), 1,2-dimyristoyl-sn-glycero-3-phosphoryl-3′-rac-glycerol (DMPG), and cholesterol.

According to an embodiment of the application, the antibody response comprises a specific IgG antibody response directed against the pTau. Preferably, the specific IgG antibody response has an anti-pTau IgG titer at least 50, 60, 70, 80, 90, 100, or more times higher than that of a placebo control.

In another embodiment of the application, the antibody response comprises a specific IgM antibody response directed against the pTau and a class switch of the specific IgM antibody response to a specific IgG antibody response directed against the pTau.

In yet another embodiment of the application, the antibody response comprises an IgG antibody response that preferentially recognizes the pTau over non-phosphorylated Tau protein. Preferably, the ratio of the anti-pTau IgG titer to the anti-Tau IgG titer is at least 5, 10, 15, 20, 25, 30, 35, 40, 45, 50, 55, 60, 65 or 70, or more.

In another embodiment of the application, the antibody response comprises an IgG antibody response against an enriched Paired Helical Filament (ePHF). Preferably, the IgG antibody response has an anti-ePHF IgG titer at least 2, 3, 4, 5, 6, 7, 8, 9, 10, 11, 12, 13, 14, 15, 16, 17, 18, 19, 20 or more times higher than that of a placebo control. More preferably, the anti-ePHF IgG has an increased binding avidity to the pathological ePHF Tau for at least 6 weeks after the initial administration of the effective amount of the liposome or after a boosting administration as measured at least 2 weeks after the boosting administration, preferably the anti-ePHF IgG has an avidity index of at least 0.3, 0.4, 0.5, 0.6, or 0.7.

According to embodiments of the application, the antibody response can be boosted by a booster administration.

In one embodiment, a method of the application further comprises administering to the subject a second dose of the effective amount of liposome 4 to 12 weeks, such as 8 weeks, after the initial administration of the effective amount of liposome. The antibody response is boosted as measured at least 2 weeks after the administration of the second dose of the effective amount of liposome. Preferably, the antibody response is boosted at least 10%, 20%, 30%, 40%, 50%, 60%, 70%, 80%, 90%, 100% or more as measured at least 2 weeks after the administration of the second dose of the effective amount of liposome.

In another embodiment, a method of the application further comprises administering to the subject a third dose of the effective amount of liposome 20 to 28 weeks, such as 24 weeks, after the initial administration of the effective amount of liposome. The antibody response is boosted as measured at least 2 weeks after the administration of the third dose of the effective amount of liposome, preferably the antibody response is increased at least 10%, 20%, 30%, 40%, 50%, 60%, 70%, 80%, 90%, 100% or more as measured at least 2 weeks after the administration of the third dose of the effective amount of liposome.

In yet another embodiment, a method of the application further comprises administering to the subject a fourth dose of the effective amount of liposome 44 to 52 weeks, such as 48 weeks, after the initial administration of the effective amount of liposome. The antibody response is boosted at least 2 weeks after the administration of the fourth dose of the effective amount of liposome, preferably the antibody response is increased at least 10%, 20%, 30%, 40%, 50%, 60%, 70%, 80%, 90%, 100% or more by the administration of the fourth dose of the effective amount of liposome.

In certain embodiments, the human subject is in need of clearance of aggregates of Tau. In certain embodiments, the subject is in need of a prevention or treatment of Alzheimer's Disease, such as preclinical Alzheimer's Disease, early Alzheimer's Disease, mild cognitive impairment (MCI) due to Alzheimer's Disease, mild Alzheimer's Disease, or mild to moderate Alzheimer's Disease. In other embodiments, the subject is amyloid positive in the brain but does not yet show significant cognitive impairment.

The invention also relates to a vaccine combination for use in inducing an immune response, such as an antibody response against a phospho-Tau protein (pTau), in a human subject in need thereof, wherein the vaccine combination comprises a primer vaccine and a booster vaccine according to embodiments of the invention, and the immune response lasts at least 10 weeks, such as at least 10, 15, 20, 25, 30, 35, 40, 45, 50 weeks or more after the administration of the primer vaccine to the human subject.

Further aspects, features and advantages of the present invention will be better appreciated upon a reading of the following detailed description of the invention and claims.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

FIG. 1 is an overview of the study design for cohort 1 (ACI-35.030 or placebo) in a Phase 1b/2a study (NCT04445831).

FIG. 2 shows the geometric mean of anti-pTau IgG titers in patients with early Alzheimer's Disease who were treated with placebo or ACI-35.030 at 300 μg or 900 μg doses at weeks 0, 8, and 24.

FIG. 3 shows the geometric mean of anti-Tau (non-phosphorylated) IgG titers in patients with early Alzheimer's Disease who were treated with placebo or ACI-35.030 at 300 μg or 900 μg doses at weeks 0, 8, and 24.

FIG. 4 shows the geometric mean of anti-ePHF IgG titers in patients with early Alzheimer's Disease who were treated with placebo or ACI-35.030 at 300 μg or 900 μg doses at weeks 0, 8, and 24.

FIG. 5 shows the geometric mean of anti-Tau (non-phosphorylated) IgG titers in patients with early Alzheimer's Disease who were treated with placebo or ACI-35.030 at 300 μg, 900 μg or 1800 μg doses.

FIG. 6 shows the geometric mean of anti-pTau IgG titers in patients with early Alzheimer's Disease who were treated with placebo or ACI-35.030 at 300 μg, 900 μg or 1800 μg doses.

FIG. 7 shows the geometric mean of anti-ePHF IgG titers in patients with early Alzheimer's Disease who were treated with placebo or ACI-35.030 at 300 μg, 900 μg or 1800 μg doses.

FIGS. 8A and 8B show the epitope recognition profile of antibodies induced in patients with early Alzheimer's Disease who were treated with placebo or ACI-35.030 at a 900 μg dose at weeks 0, 8, and 24.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The invention provides a method of inducing anti-phosphorylated Tau antibodies without inducing a severe adverse event considered either possibly or probably related to the study vaccine, such as encephalitis, in a human subject in need thereof. In particular embodiments, the method comprises administering to the subject an effective amount of liposomes comprising a Tau phosphopeptide presented on the surface of the liposome and a toll-like receptor 4 agonist.

As used herein, the term “anti-phosphorylated Tau antibody” refers to an antibody that binds to Tau that has been phosphorylated on an amino acid residue at one or more locations of the amino acid sequence of Tau. The phosphorylated amino acid residues can be, e.g., serine (Ser), threonine (Thr) or tyrosine (Tyr). The site on phosphorylated Tau to which the anti-phosphorylated Tau antibody binds is preferably a site that is specifically phosphorylated in neurodegenerative diseases such as Alzheimer's Disease. Examples of sites of phosphorylated Tau to which the anti-phosphorylated Tau antibody binds include, for example, Tyr18, Ser199, Ser202, Thr205, Thr212, Ser214, Ser396, Ser404, Ser409, Ser422, Thr427. As used throughout the present application, the amino acid positions are given in reference to the sequence of human microtubule-associated protein tau isoform 2 having the amino acid sequence represented in GenBank Accession No. NP_005901.2.

The ability to induce anti-phosphorylated Tau antibodies upon administration can be determined by testing a biological sample (e.g., blood, plasma, serum, PBMCs, urine, saliva, feces, CSF or lymph fluid) from the subject for the presence of antibodies, e.g., IgG or IgM antibodies, directed to the immunogenic Tau peptide(s) administered in the pharmaceutical composition (see for example Harlow, 1989, Antibodies, Cold Spring Harbor Press). For example, titers of antibodies produced in response to administration of a composition providing an immunogen can be measured by enzyme-linked immunosorbent assay (ELISA), other ELISA-based assays (e.g., MSD-Meso Scale Discovery), dot blots, SDS-PAGE gels, ELISPOT or Antibody-Dependent Cellular Phagocytosis (ADCP) Assay.

As used herein, the term “adverse event” (AE) refers to any untoward medical occurrence in a patient administered a pharmaceutical product and which does not necessarily have a causal relationship with the treatment. According to embodiments of the invention, AEs are rated on a 3-point scale of increasing severity using the following definitions: mild (grade 1), referring to an AE that is easily tolerated by the subject, which causes minimal discomfort and does not interfere with everyday activities; moderate (grade 2), referring to an AE that is sufficiently discomforting to interfere with normal everyday activities and intervention may be needed; severe (grade 3), referring to an AE that prevents normal everyday activities, and treatment or other intervention is usually needed. A serious AE (SAE) can be any AE occurring at any dose that results in any of the following outcomes: death, where death is an outcome, not an event; life-threatening, referring to an event in which the patient is at risk of death at the time of the event; it does not refer to an event which could hypothetically have caused death had it been more severe; in patient hospitalization, i.e., an unplanned, overnight hospitalization, or prolongation of an existing hospitalization; persistent or significant incapacity or substantial disruption of the ability to conduct normal life functions; congenital anomaly/birth defect; important medical event (as deemed by the investigator) that may jeopardize the patients or may require medical or surgical intervention to prevent one of the other outcomes listed above (e.g., intensive treatment in an emergency room or at home for allergic bronchospasm or blood dyscrasias or convulsions that do not result in hospitalization). Hospitalization is official admission to a hospital. Hospitalization or prolongation of a hospitalization constitutes criteria for an AE to be serious; however, it is not in itself considered an SAE. In the absence of an AE, hospitalization or prolongation of hospitalization should not be reported as a SAE by the participating investigator. This can be the case, in the following situations: the hospitalization or prolongation of hospitalization is needed for a procedure required by the protocol; or the hospitalization or prolongation of hospitalization is a part of a routine procedure followed by the center (e.g., stent removal after surgery). This should be recorded in the study file. Hospitalization for elective treatment of a pre-existing condition that did not worsen during the study is not considered an AE.

Complications that occur during hospitalization are AEs. If a complication prolongs hospitalization, or meets any of the other SAE criteria, then the event is an SAE.

As used herein, the term “encephalitis” refers to an inflammation of the brain which can result from infectious and non-infectious causes. As used herein, the term “meningoencephalitis” refers to a condition characterized by infection or inflammation of the brain meninges and of the brain. The diagnosis of encephalitis or meningoencephalitis can be determined by techniques known to those skilled in the art in view of the present disclosure, for example, by clinical, neurological and psychiatric examinations, biological sampling including blood and CSF samplings, MRI scanning and electroencephalography (EEG).

As used herein, the term “liposome” refers generally to a lipid vesicle that is made of materials having high lipid content, e.g., phospholipids, cholesterol. The lipids of these vesicles are generally organized in the form of lipid bilayers. The lipid bilayers generally encapsulate a volume which is either interspersed between multiple onion-like shells of lipid bilayers, forming multilamellar lipid vesicles (MLVs) or contained within an amorphous central cavity. Lipid vesicles having an amorphous central cavity are unilamellar lipid vesicles, i.e., those with a single peripheral bilayer surrounding the cavity. Large unilamellar vesicles (LUVs) generally have a diameter of 100 nm to few micrometer, such as 100-200 nm or larger, while small unilamellar lipid vesicles (SUV) generally have a diameter of less than 100 nm, such as 20-100 nm, typically 15-30 nm.

As used herein, the term “Tau” or “Tau protein”, also known as microtubule-associated protein Tau, MAPT, neurofibrillary tangle protein, paired helical filament-Tau, PHF-Tau, MAPTL, MTBT1, refers to an abundant central and peripheral nervous system protein having multiple isoforms. In the human central nervous system (CNS), six major Tau isoforms ranging in size from 352 to 441 amino acids in length exist due to alternative splicing (Hanger et al., Trends Mol Med. 15:112-9, 2009). Examples of Tau include, but are not limited to, Tau isoforms in the CNS, such as the 441-amino acid longest Tau isoform (4R2N), also named microtubule-associated protein tau isoform 2, that has four repeats and two inserts, such as the human Tau isoform 2 having the amino acid sequence represented in GenBank Accession No. NP_005901.2. Other examples of Tau include the 352-amino acid long shortest (fetal) isoform (3R0N), also named microtubule-associated protein tau isoform 4, that has three repeats and no inserts, such as the human Tau isoform 4 having the amino acid sequence represented in GenBank Accession No. NP_058525.1. Examples of Tau also include the “big Tau” isoform expressed in peripheral nerves that contains 300 additional residues (exon 4a). Friedhoff et al., Biochimica et Biophysica Acta 1502 (2000) 122-132. Examples of Tau include a human big Tau that is a 758 amino acid-long protein encoded by an mRNA transcript 6762 nucleotides long (NM_016835.4), or isoforms thereof. The amino acid sequence of the exemplified human big Tau is represented in GenBank Accession No. NP_058519.3. As used herein, the term “Tau” includes homologs of Tau from species other than human, such as Macaca Fascicularis (cynomolgus monkey), rhesus monkeys or Pan troglodytes (chimpanzee). As used herein, the term “Tau” includes proteins comprising mutations, e.g., point mutations, fragments, insertions, deletions and splice variants of full-length wild type Tau. The term “Tau” also encompasses post-translational modifications of the Tau amino acid sequence. Post-translational modifications include, but are not limited to, phosphorylation.

As used herein, the term “peptide” or “polypeptide” refers to a polymer composed of amino acid residues, related naturally occurring structural variants, and synthetic non-naturally occurring analogs thereof linked via peptide bonds. The term refers to a peptide of any size, structure, or function. Typically, a peptide is at least three amino acids long. A peptide can be naturally occurring, recombinant, or synthetic, or any combination thereof. Synthetic peptides can be synthesized, for example, using an automated polypeptide synthesizer. Examples of Tau peptides include any peptide of Tau protein of about 5 to about 30 amino acids in length, preferably of about 10 to about 25 amino acids in length, more preferably of about 16 to about 21 amino acids in length. In the present disclosure, peptides are listed from N to C terminus using the standard three or one letter amino acid abbreviation, wherein phosphoresidues are indicated with “p”. Examples of Tau peptides useful in the invention include, but are not limited to, Tau peptides comprising the amino acid sequence of any of SEQ ID NOs: 1-12, or Tau peptides having an amino acid sequence that is at least 75%, 80%, 85%, 90% or 95% identical to the amino acid sequence of any of SEQ ID NOs: 1-12.

As used herein, the term “phosphopeptide” or “phospho-epitope” refers to a peptide that is phosphorylated at one or more amino acid residues. Examples of Tau phosphopeptides include any Tau peptide comprising one or more phosphorylated amino acid residues.

The Tau peptides of the present invention can be synthesized by solid phase peptide synthesis or by recombinant expression systems. Automatic peptide synthesizers are commercially available from numerous suppliers, such as Applied Biosystems (Foster City, Calif.). Recombinant expression systems can include bacteria, such as E. coli, yeast, insect cells, or mammalian cells. Procedures for recombinant expression are described by Sambrook et al., Molecular Cloning: A Laboratory Manual (C.S.H.P. Press, NY 2d ed., 1989).

According to particular embodiments, the liposome comprises one or more Tau peptides. According to particular embodiments, the Tau peptides in the liposome can be the same or different. Any suitable Tau peptide known to those skilled in the art can be used in the invention in view of the present disclosure. According to particular embodiments, one or more of the Tau peptides comprise the amino acid sequence of one of SEQ ID NOs: 1-12. In other embodiments, one or more of the Tau peptides comprise an amino acid sequence that is at least 75%, 80%, 85%, 90% or 95% identical to the amino acid sequence of one of SEQ ID NOs: 1-12, wherein none of the amino acid residues are phosphorylated, or one or more amino acid residues are phosphorylated.

According to particular embodiments, one or more of the Tau peptides are Tau phosphopeptides. According to particular embodiments, the one or more Tau phosphopeptides comprise the amino acid sequence of one of SEQ ID NOs: 1-3 or 5-12, or an amino acid sequence that is at least 75%, 80%, 85%, 90% or 95% identical to the amino acid sequence of one of SEQ ID NOs: 1-3 or 5-12, wherein one or more of the indicated amino acid residues are phosphorylated. Preferably, the Tau phosphopeptide comprises the amino acid sequence of one of SEQ ID Nos: 1-3. The Tau peptide can have the C-terminus amidated.

According to embodiments of the application, a Tau peptide is presented on the surface of the liposome. A Tau peptide, preferably a Tau phosphopeptide, can be presented on the surface of the liposome using methods known in the art in view of the present disclosure. See, for example, the relevant disclosure in U.S. Pat. Nos. 8,647,631 and 9,687,447, and International Patent Application No. PCT/US18/57286, the content of which is incorporated herein by reference. According to particular embodiments, the one or more Tau peptides, including phosphopeptides, further comprise one or more modifications, such as palmitoylation or dodecyl modification to allow the Tau peptides to be presented on the surface of the liposome. Additional amino acid residues, such as Lys, Cys, or sometimes Ser or Thr, can be added to the Tau peptide to facilitate the modification. It was reported that the position of lipid anchors induces different conformations of the peptide sequence (Hickman et al., J. Biol. Chem. vol. 286, No. 16, pp. 13966-13976, Apr. 22, 2011). While not wishing to be bound by theory, it is believed that adding hydrophobic moieties at both termini may increase the pathological beta-sheet conformation of the Tau peptide. Thus, the one or more Tau peptides further comprise hydrophobic moieties at both termini. The modified Tau peptide can have the C-terminus amidated. Preferably, a Tau peptide presented on the surface of the liposome consists of the amino acid sequence of one of SEQ ID NO:27 to SEQ ID NO:29 and SEQ ID NO:31 to SEQ ID NO: 38.

Examples of tau liposomes useful for the present invention include, but are not limited, tau liposomes described in U.S. Pat. Nos. 8,647,631 and 9,687,447, and International Patent Application No. PCT/US18/57286, the disclose of each is herein incorporated by reference in its entirety.

As used herein, the term “effective amount” refers to an amount of an active ingredient or component that elicits the desired biological or medicinal response in a subject. Selection of a particular effective dose can be determined (e.g., via clinical trials) by those skilled in the art based upon the consideration of several factors, including the disease to be treated or prevented, the symptoms involved, the patient's body mass, the patient's immune status and other factors known by the skilled artisan. The precise dose to be employed in the formulation will also depend on the mode of administration, route of administration, target site, physiological state of the patient, other medications administered and the severity of disease, and should be decided according to the judgment of the practitioner and each patient's circumstances. For example, the effective amount of tau phosphopeptide also depends on whether adjuvant is also administered, with higher dosages being required in the absence of adjuvant. Effective doses can be extrapolated from dose-response curves derived from in vitro or animal model test systems.

According to embodiments of the application, an effective amount of liposomes comprises an amount of Tau phosphopeptide that is sufficient to increase a level of anti-phosphorylated Tau antibodies, without inducing a severe adverse event, such as encephalitis. In particular embodiments, an effective amount of liposomes comprises a Tau phosphopeptide at an amount of about 25 nmoles to about 750 nmoles per dose, such as about 29.7 nmoles to about 742.5 nmoles per dose, preferably about 90 nmoles to about 715 nmoles per dose, such as about 89.1 nmoles to about 712.8 nmoles per dose, or about 90 nmoles to about 535 nmoles per dose, such as about 89.1 nmoles to about 534.6 nmoles per dose, or about 90 nmoles to about 275 nmoles per dose, such as about 89.1 nmoles to about 267.3 nmoles per dose. The amount of Tau phosphopeptide administered can also be expressed by weight. For example, 29.7 nmoles per dose corresponds to 100 μg per dose of a tetrapalmitoylated Tau phosphopeptide consisting of the amino acid sequence of SEQ ID NO: 28, 742.5 nmoles per dose corresponds to 2500 μg per dose of a tetrapalmitoylated Tau phosphopeptide consisting of the amino acid sequence of SEQ ID NO: 28, 89.1 nmoles per dose corresponds to 300 μg per dose of a tetrapalmitoylated Tau phosphopeptide consisting of the amino acid sequence of SEQ ID NO: 28, 712.8 nmoles per dose corresponds to 2400 μg per dose of a tetrapalmitoylated Tau phosphopeptide consisting of the amino acid sequence of SEQ ID NO: 28, and 534.6 nmoles per dose corresponds to 1800 μg per dose of a tetrapalmitoylated Tau phosphopeptide consisting of the amino acid sequence of SEQ ID NO: 28. The tetrapalmitoylated Tau phosphopeptide has four lipidic chains that allow the presentation of the Tau phosphopeptide on the surface of the liposomes. The doses of 300, 900, 1800 μg of tetrapalmitoylated Tau phosphopeptide consisting of the amino acid sequence of SEQ ID NO: 28 correspond to 169, 508, 1016 μg, respectively of the corresponding “naked” peptide without any of the lipidic chains.

According to embodiments of the application, an effective amount of liposomes comprises a Tau phosphopeptide at an amount of about 25 nmoles to about 750 nmoles per dose, such as about 25 nmoles, about 30 nmoles, about 35 nmoles, about 40 nmoles, about 45 nmoles, about 50 nmoles, about 55 nmoles, about 60 nmoles, about 65 nmoles, about 70 nmoles, about 75 nmoles, about 80 nmoles, about 85 nmoles, about 90 nmoles, about 95 nmoles, about 100 nmoles, about 125 nmoles, about 150 nmoles, about 175 nmoles, about 200 nmoles, about 225 nmoles, about 250 nmoles, about 275 nmoles, about 300 nmoles, about 325 nmoles, about 350 nmoles, about 375 nmoles, about 400 nmoles, about 425 nmoles, about 450 nmoles, about 475 nmoles, about 500 nmoles, about 525 nmoles, about 550 nmoles, about 575 nmoles, about 600 nmoles, about 625 nmoles, about 650 nmoles, about 675 nmoles, about 700 nmoles, about 725 nmoles, about 750 nmoles per dose of a Tau phosphopeptide comprising the amino acid sequence of one of SEQ ID NOs: 1-3 or 5-12. Preferably, the Tau phosphopeptide consists of the amino acid sequence of one of SEQ ID NO:27 to SEQ ID NO:29 and SEQ ID NO:31 to SEQ ID NO: 38. More preferably, the Tau phosphopeptide consists of the amino acid sequence of SEQ ID NO: 28.

According to embodiments of the application, an effective amount of liposomes comprises a tetrapalmitoylated Tau phosphopeptide at an amount of 100 μg to 2500 μg, 300 μg to 2400 μg, 300 μg to 1800 μg, or 300 μg to 900 μg per dose, such as 100 μg, 150 μg, 200 μg, 250 μg, 300 μg, 400 μg, 500 μg, 600 μg, 700 μg, 800 μg, 900 μg, 1000 μg, 1100 μg, 1200 μg, 1300 μg, 1400 μg, 1500 μg, 1600 μg, 1700 μg, 1800 μg, 1900 μg, 2000 μg, 2100 μg, 2200 μg, 2300 μg, 2400 μg, or 2500 μg per dose.

According to embodiments of the application, the Tau phosphopeptide is presented on the surface of the liposomes. According to embodiments of the application, the Tau phosphopeptide comprises the amino acid sequence of one of SEQ ID NOs: 1-3 or 5-12. Preferably, the Tau phosphopeptide consists of the amino acid sequence of one of SEQ ID NO:27 to SEQ ID NO:29 and SEQ ID NO:31 to SEQ ID NO:38. More preferably, the Tau phosphopeptide consists of the amino acid sequence of SEQ ID NO:28.

According to other embodiments of the application, an effective amount of liposomes further comprises a toll-like receptor 4 agonist at an amount of 30 μg to 900 μg, preferably 100 μg to 585 μg, per dose. For example, the effective amount of liposomes can comprise a toll-like receptor 4 agonist at an amount of 30 μg, 50 μg, 100 μg, 150 μg, 200 μg, 250 μg, 300 μg, 330 μg, 360 μg, 390 μg, 420 μg, 450 μg, 480 μg, 500 μg, 520 μg, 540 μg, 560 μg, 580 μg, 600 μg, 700 μg, 800 μg or 900 μg per dose.

According to embodiments of the application, the toll-like receptor 4 comprises 3D-(6-acyl) PHAD®.

According to other embodiments of the application, an effective amount of liposomes further comprises a helper T-cell epitope at an amount of 25 μg to 625 μg, preferably 75 μg to 550 μg, such as 75 μg to 450 μg, 80 μg to 540 μg, 82.5 μg to 535 μg, 85 μg to 530 μg, 87.5 μg to 525 μg, or 90 μg to 520 μg, per dose. For example, the effective amount of liposomes can comprise a helper T-cell epitope at an amount of 25 μg, 50 μg, 70 μg, 72.5 μg, 75 μg, 77.5 μg, 80 μg, 82.5 μg, 85 μg, 87.5 μg, 90 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 550 μg, 575 μg, 600 μg, or 625 μg per dose.

According to other embodiments of the application, an effective amount of liposomes further comprises a helper T-cell epitope at an amount of about 3 nmoles to about 105 nmoles per dose, such as about 4 nmoles, about 5 nmoles, about 6 nmoles, about 7 nmoles, about 8 nmoles, about 9 nmoles, about 10 nmoles, about 15 nmoles, about 20 nmoles, about 25 nmoles, about 30 nmoles, about 35 nmoles, about 40 nmoles, about 45 nmoles, about 50 nmoles, about 55 nmoles, about 60 nmoles, about 65 nmoles, about 70 nmoles, about 75 nmoles, about 80 nmoles, about 85 nmoles, about 90 nmoles, about 95 nmoles, about 100 nmoles, or about 105 nmoles per dose.

According to embodiments of the application, the helper T-cell epitope is a T50 helper T-cell epitope consisting of the amino acid sequence of SEQ ID NO: 13, a T46 helper T-cell epitope consisting of the amino acid sequence of SEQ ID NO: 14, a T48 helper T-cell epitope consisting of the amino acid sequence of SEQ ID NO: 15, a T51 helper T-cell epitope consisting of the amino acid sequence of SEQ ID NO: 16, or a T52 helper T-cell epitope consisting of the amino acid sequence of SEQ ID NO: 17, preferably the helper T-cell epitope is a T50 helper T-cell epitope consisting of the amino acid sequence of SEQ ID NO: 13.

In certain embodiments, an effective amount of liposomes further comprises a lipidated CpG oligonucleotide at an amount of 50 μg to 1250 μg, preferably 100 μg to 1000 μg, such as 150 μg to 800 μg, 150-900 μg, 125 μg to 950 μg or 150 μg to 850 μg per dose. For example, the effective amount of liposomes can comprise a lipidated CpG oligonucleotide at an amount of 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1200 μg, or 1250 μg per dose.

According to embodiments of the application, the lipidated CpG oligonucleotide is a CpG oligonucleotide comprising a nucleotide sequence of one of SEQ ID NOs: 18-22, preferably the lipidated CpG oligonucleotide is a CpG oligonucleotide comprising a nucleotide sequence of SEQ ID NO: 18. According to embodiments of the application, the lipidated CpG oligonucleotide is a CpG oligonucleotide comprising a nucleotide sequence of SEQ ID NO: 18 which has one or more phosphorothioate internucleotide linkages and is covalently linked to cholesterol via a linker comprising polyethylene glycol (PEG).

According to embodiments, the effective amount of liposomes comprise 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1200 μg, or 1250 μg per dose of the CpG oligonucleotide covalently linked to cholesterol via the PEG linker.

As used herein a “sustained immune response” or a “sustainable immune response” refers to an immune response that lasts at least six weeks after the initial administration of an effective amount of a liposome. According to embodiments of the application, a “sustained immune response” is a sustained antibody response that lasts at least six weeks, at least 12 week, at least 24 weeks, at least 36 weeks, at least 48 weeks, at least 60 weeks, at least 72 weeks or longer, and the antibody response can be characterized by the presence of anti-phosphorylated Tau IgG, anti-phosphorylated Tau IgM, or anti-ePHF. Anti-phosphorylated Tau IgG, anti-phosphorylated Tau IgM and anti-ePHF can be detected and measured by any method known to one of skill in the art, including those described herein.

As used herein an “antibody response that lasts” refers to an antibody response that is maintained at a level equal to or higher than a defined threshold level during a specified period of time after the initial administration of an effective amount of a liposome, and the defined threshold level is higher than a baseline level measured before the initial administration of the effective amount of the liposome. In some embodiments, the baseline level is determined based on the average measured level of antibody titers before the initial administration, preferably two measurements are performed. In one embodiment, the antibody response comprises a specific IgG antibody response directed against the pTau, and the defined threshold level is at least 1.5 or more times of the baseline level, such as at least 1.5, 1.6, 1.7, 1.8, 1.9, 2.0 or more times of the baseline level. In another embodiment, the antibody response comprises an IgG immune response against ePHF, and the defined threshold level is at least 2.0 or more times of the baseline level, such as at least 2.0, 2.1, 2.2, 2.3, 2.4, 2.5 or more times of the baseline level.

According to particular embodiments, the human subject is in need of treatment of a neurodegenerative disease, disorder, or condition.

As used herein a “neurodegenerative disease, disorder, or condition” includes any neurodegenerative disease, disorder, or condition known to those skilled in the art in view of the present disclosure. Examples of neurodegenerative diseases, disorders, or conditions include neurodegenerative diseases or disorders caused by or associated with the formation of neurofibrillary lesions, such as Tau-associated diseases, disorders or conditions, referred to as Tauopathies. According to particular embodiments, the neurodegenerative disease, disorder, or condition includes any of the diseases or disorders which show co-existence of Tau and amyloid pathologies including, but not is limited to, Alzheimer's Disease, Parkinson's Disease, Creutzfeldt-Jacob disease, Dementia pugilistica, Down Syndrome, Gerstmann-Sträussler-Scheinker disease, inclusion body myositis, prion protein cerebral amyloid angiopathy, traumatic brain injury, amyotrophic lateral sclerosis, parkinsonism-dementia complex of Guam, Non-Guamanian motor neuron disease with neurofibrillary tangles, argyrophilic grain dementia, corticobasal degeneration, Dementia Lewy Amyotrophic Lateral sclerosis, diffuse neurofibrillary tangles with calcification, frontotemporal dementia, preferably frontotemporal dementia with parkinsonism linked to chromosome 17 (FTDP-17), frontotemporal lobar dementia, Hallervorden-Spatz disease, multiple system atrophy, Niemann-Pick disease type C, Pick's disease, progressive subcortical gliosis, progressive supranuclear palsy, Subacute sclerosing panencephalitis, Tangle only dementia, Postencephalitic Parkinsonism, Myotonic dystrophy, chronic traumatic encephalopathy (CTE), Primary age-related Tauopathy (PART), cerebral angiopathy or Lewy body dementia (LBD). According to particular embodiments, the neurodegenerative disease, disorder, or condition is Alzheimer's Disease or another Tauopathy. According to preferred embodiments, the neurodegenerative disease, disorder, or condition is Alzheimer's Disease.

The clinical course of Alzheimer's Disease can be divided into stages, with progressive patterns of cognitive and functional impairments. The stages can be defined using grading scales known in the art including, e.g., NIA-AA Research Framework. See, e.g., Dubois et al., Alzheimer's & Dementia 12 (2016) 292-323, Dubois et al., Lancet Neurol 2014; 13: 614-29, Jack et al., Alzheimer's & Dementia 14 (2018) 535-562, the content of each of which is hereby incorporated by references in its entirety.

According to preferred embodiments, the neurodegenerative disease, disorder, or condition is early Alzheimer's Disease, mild cognitive impairment (MCI) due to Alzheimer's Disease, mild Alzheimer's Disease, or mild to moderate Alzheimer's Disease.

In some embodiments, the subject in need of a treatment is amyloid positive in the brain but does not yet show significant cognitive impairment. The amyloid deposition in the brain can be detected using methods known in the art, such as PET scan, immunoprecipitation mass spectrometry or other methods.

As used herein, the term “toll-like receptor” or “TLR” refers to a class of pattern recognition receptor (PRR) proteins that play a key role in the innate immune response. TLRs recognize pathogen-associated molecular patterns (PAMPs) from microbial pathogens, such as bacteria, fungi, parasites and viruses, which can be distinguished from host molecules. TLRs are membrane-spanning proteins that typically function as dimers and are expressed by cells involved in the innate immune response, including antigen-presenting dendritic cells and phagocytic macrophages. There are at least ten human TLR family members, TLR1 to TLR10, and at least twelve murine TLR family members, TLR1 to TLR9 and TLR11 to TLR13, and they differ in the types of antigens they recognize. For example, TLR4 recognizes lipopolysaccharides (LPS), a component present in many Gram-negative bacteria, as well as viral proteins, polysaccharide, and endogenous proteins such as low-density lipoprotein, beta-defensins and heat shock protein; and TLR9 is a nucleotide-sensing TLR which is activated by unmethylated cytosine-phosphate-guanine (CpG) single-stranded or double-stranded dinucleotides, which are abundant in prokaryotic genomes but rare in vertebrate genomes. Activation of TLRs leads to a series of signaling events resulting in the production of type I interferons (IFNs), inflammatory cytokines, and chemokines, and the induction of immune responses. Eventually, this inflammation also activates the adaptive immune system, which then results in the clearance of the invading pathogens and the infected cells.

As used herein, the term “agonist” refers to a molecule that binds to one or more TLRs and induces a receptor mediated response. For example, an agonist can induce, stimulate, increase, activate, facilitate, enhance, or up regulate the activity of the receptor. Such activities are referred to as “agonistic activities.” For example, a TLR4 or TLR9 agonist can activate or increase cell signaling through the bound receptor. Agonists include, but are not limited to nucleic acids, small molecules, proteins, carbohydrates, lipids or any other molecules that bind or interact with receptors. Agonists can mimic the activity of a natural receptor ligand. Agonists can be homologous to these natural receptor ligands with respect to sequence, conformation, charge or other characteristics such that they can be recognized by the receptors. This recognition can result in physiologic and/or biochemical changes within the cell, such that the cell reacts to the presence of the agonist in the same manner as if the natural receptor ligand were present. According to particular embodiments, the toll-like receptor agonist is at least one of a toll-like receptor 4 agonist and a toll-like receptor 9 agonist.

As used herein, the terms “induce” and “stimulate” and variations thereof refer to any measurable increase in cellular activity. Induction of an immune response can include, for example, activation, proliferation, or maturation of a population of immune cells, increasing the production of a cytokine, and/or another indicator of increased immune function. In certain embodiments, induction of an immune response can include increasing the proliferation of B cells, producing antigen-specific antibodies, increasing the proliferation of antigen-specific T cells, improving dendritic cell antigen presentation and/or an increasing expression of certain cytokines, chemokines and co-stimulatory markers.

As used herein, the term “toll-like receptor 4 agonist” refers to any compound that acts as an agonist of TLR4. Any suitable toll-like receptor 4 agonist known to those skilled in the art in view of the present disclosure can be used in the invention. Examples of toll-like receptor 4 ligand useful for the invention include TLR4 agonist, including, but not limited to, monophosphoryl lipid A (MPLA). As used herein, the term “monophosphoryl lipid A” or “MPLA” refers to a modified form of lipid A, which is the biologically active part of Gram-negative bacterial lipopolysaccharide (LPS) endotoxin. MPLA is less toxic than LPS while maintaining the immunostimulatory activity. As a vaccine adjuvant, MPLA stimulates both cellular and humoral responses to the vaccine antigen. Examples of MPLA include, but are not limited to, 3-O-desacyl-4′-monophosphoryl lipid A, Monophosphoryl Hexa-acyl Lipid A, 3-Deacyl (Synthetic) (also referred to as 3D-(6-acyl) PHAD®), monophosphoryl 3-deacyl lipid A, and structurally related variants thereof. MPLA useful for the invention can be obtained using methods known in the art, or from a commercial source, such as 3D-(6-acyl) PHAD®, PHAD®, PHAD®-504, 3D-PHAD® from Avanti Polar Lipids (Alabaster, Alabama, USA) or MPL™ from various commercial sources. According to particular embodiments, the toll-like receptor 4 agonist is MPLA. According to particular embodiments, the liposome comprising a Tau phosphopeptide and a toll-like receptor 4 agonist also comprises a helper T-cell epitope that is capable of binding most or all HLA DR (Human Leukocyte Antigen-antigen D Related) molecules. The helper T-cell epitope is then able to activate CD4⁺ T-cells and provides essential maturation and survival signals to the Tau-specific B-cells. The Tau liposomes can be used to generate high-quality antibodies against the pTau antigen in homologous or heterologous immunization schemes, with the liposome used in the prime and/or in the boost.

As used herein, the term “helper T-cell epitope” refers to a polypeptide comprising an epitope that is capable of recognition by a helper T-cell. Examples of helper T-cell epitopes include, but are not limited to, tetanus toxoid (e.g., the P2 and P30 epitopes, also named, respectively as T2 and T30), Hepatitis B surface antigen, cholera toxin B, diphtheria toxoid, measles virus F protein, Chlamydia trachomatis major outer membrane protein, Plasmodium falciparum circumsporozite T, P. falciparum CS antigen, Schistosoma mansoni triose phosphate isomerase, Bordetella pertussis, Clostridium tetani, Pertusaria trachythallina, Escherichia coli TraT, and Influenza virus hemagglutinin (HA).

Any suitable helper T-cell epitope known to those skilled in the art can be used in the invention in view of the present disclosure. According to particular embodiments, the helper T-cell epitope comprises at least one amino acid sequence selected from the group consisting of SEQ ID NO:23 to SEQ ID NO:26. Preferably, the helper T-cell epitope comprises two or more of the amino acid sequences of SEQ ID NO:23 to SEQ ID NO:26 fused together via a linker, such as a peptide linker comprising one or more amino acids, e.g., Val (V), Ala (A), Arg (R), Gly (G), Ser(S), Lys (K). The length of the linker can vary, preferably 1-5 amino acids. Preferably, the helper T-cell epitope comprises three or more of the amino acid sequences of SEQ ID NO:23 to SEQ ID NO:26 fused together via one or more linkers selected from the group consisting of VVR, GS, RR, RK. The helper T-cell epitope can have its C-terminus amidated.

According to embodiments of the application, the helper T-cell epitopes can be incorporated on the liposomal surface, e.g. anchored by a covalently bound hydrophobic moiety wherein said hydrophobic moiety is an alkyl group, a fatty acid, a triglyceride, diglyceride, steroid, sphingolipid, glycolipid or a phospholipid, particularly an alkyl group or a fatty acid, particularly with a carbon backbone of at least 3 carbon atoms, particularly of at least 4 carbon atoms, particularly of at least 6 carbon atoms, particularly of at least 8 carbon atoms, particularly of at least 12 carbon atoms, particularly of at least 16 carbon atoms. In one embodiment of the invention, the hydrophobic moiety is palmitic acid. Alternatively, the helper T-cell epitopes can be encapsulated in the liposomes. According to particular embodiments, the helper T-cell epitope is encapsulated in the liposome.

The helper T-cell epitope can be modified for its desired location in the liposomes using methods known in the art in view of the present disclosure. According to particular embodiments, the helper T-cell epitope useful for the invention comprises an amino acid sequence of one of SEQ ID NO:39 to SEQ ID NO:44. Preferably, the helper T cell epitope consists of an amino acid sequence selected from the group consisting of SEQ ID NO:13 to SEQ ID NO: 17.

According to particular embodiments, the liposome comprising a Tau phosphopeptide and a toll-like receptor 4 agonist also comprises a toll-like receptor 9 agonist. As used herein, the term “toll-like receptor 9 agonist” refers to any compound that acts as an agonist of TLR9. Any suitable toll-like receptor 9 agonist known to those skilled in the art in view of the present disclosure can be used in the invention. Examples of toll-like receptor 9 ligand useful for the invention include TLR9 agonist including, but not limited to, CpG oligonucleotides.

As used herein, the term “CpG oligonucleotide”, “CpG oligodeoxynucleotide” or “CpG ODN” refers to an oligonucleotide comprising at least one CpG motif. As used herein, “oligonucleotide,” “oligodeoxynucleotide” or “ODN” refers to a polynucleotide formed from a plurality of linked nucleotide units. Such oligonucleotides can be obtained from existing nucleic acid sources or can be produced by synthetic methods. As used herein, the term “CpG motif” refers to a nucleotide sequence which contains unmethylated cytosine-phosphate-guanine (CpG) dinucleotides (i.e., a cytosine (C) followed by a guanine (G)) linked by a phosphate bond or a phosphodiester backbone or other internucleotide linkages.

According to particular embodiments, the CpG oligonucleotide is lipidated, i.e., conjugated (covalently linked) to a lipid moiety.

As used herein, a “lipid moiety” refers to a moiety containing a lipophilic structure. Lipid moieties, such as an alkyl group, a fatty acid, a triglyceride, diglyceride, steroid, sphingolipid, glycolipid or a phospholipid, particularly a sterol such as cholesterol, or fatty acids, when attached to highly hydrophilic molecules, such as nucleic acids, can substantially enhance plasma protein binding and consequently circulation half-life of the hydrophilic molecules. In addition, binding to certain plasma proteins, such as lipoproteins, has been shown to increase uptake in specific tissues expressing the corresponding lipoprotein receptors (e.g., LDL-receptor HDL-receptor or the scavenger receptor SR-B1). In particular, a lipid moiety conjugated to the phosphopeptides and/or CpG oligonucleotide allows anchoring the said peptides and/or oligonucleotides into the membrane of a liposome via a hydrophobic moiety.

According to particular embodiments, in view of the present disclosure, the CpG oligonucleotide can comprise any suitable internucleotide linkages.

As used herein, the term “internucleotide linkage” refers to a chemical linkage to join two nucleotides through their sugars consisting of a phosphorous atom and a charged or neutral group between adjacent nucleosides. Examples of internucleotide linkage include phosphodiester (po), phosphorothioate (ps), phosphorodithioate (ps2), methylphosphonate (mp), and methylphosphorothioate (rp). Phosphorothioate, phosphorodithioate, methylphosphonate and methylphosphorothioate are stabilizing internucleotide linkages, while phosphodiester is a naturally-occurring internucleotide linkage. Oligonucleotide phosphorothioates are typically synthesized as a random racemic mixture of Rp and Sp phosphorothioate linkages.

Any suitable CpG oligonucleotide known to those skilled in the art can be used in the invention in view of the present disclosure. Examples of such CpG oligonucleotides include, but are not limited to CpG2006 (also known as CpG 7909) (SEQ ID NO: 18), CpG 1018 (SEQ ID NO: 19), CpG2395 (SEQ ID NO: 20), CpG2216 (SEQ ID NO: 21) or CpG2336 (SEQ ID NO: 22).

A CpG oligonucleotide can be lipidated using methods known in the art in view of the present disclosure. In some embodiments, the CpG oligonucleotide is covalently linked to a cholesterol molecule directly. In some embodiments, the 3′ terminus of a CpG oligonucleotide is covalently linked to a cholesterol molecule through a phosphate bond, optionally via a PEG linker. In some embodiments, the 5′ terminus of a CpG oligonucleotide is covalently linked to a cholesterol molecule through a phosphate bond, optionally via a PEG linker. Other lipophilic moiety can also be covalently linked to the 5′ or 3′ terminus of a CpG oligonucleotide. For example, a CpG oligonucleotide can be covalently linked to a lipid anchor of the same length as the phospholipids from liposome: one palmitic acid chain (using Pal-OH or similar, activated for coupling) or two palmitic acids (e.g., using 1,2-dipalmitoyl-sn-glycero-3-phosphoethanolamine-N-(succinyl) or similar, activated for coupling), optionally via a PEG linker. See, e.g., relevant disclosure in U.S. Pat. No. 7,741,297, the content of which is incorporated herein by reference. The length of PEG can vary, from example, from 1 to 5 PEG units.

Other linkers can also be used to covalently connect a CpG oligonucleotide to a lipophilic moiety (such as a cholesterol molecule), examples of which include, but are not limited to an alkyl spacer having 3 to 12 carbons. A short linker compatible with oligonucleotide chemistry is needed as aminodiol. In some embodiment, no linker is used for the covalent bonding. See e.g., Ries et al., “Convenient synthesis and application of versatile nucleic acid lipid membrane anchors in the assembly and fusion of liposomes,” Org. Biomol. Chem., 2015, 13, 9673, the relevant disclosure of which is incorporated herein by reference.

According to particular embodiments, lipidated CpG oligonucleotide useful for the invention comprises a nucleotide sequence selected from the group consisting of SEQ ID NO:18 to SEQ ID NO:22, wherein the nucleotide sequence comprises one or more phosphorothioate internucleotide linkages, and the nucleotide sequence is covalently linked to at least one cholesterol via a linker. According to preferred embodiments, the lipidated CpG oligonucleotide comprises a nucleotide sequence of SEQ ID NO: 18, has one or more phosphorothioate internucleotide linkages, and is covalently linked to cholesterol. Any suitable linkers can be used to covalently link a CpG oligonucleotide to a cholesterol molecule. Preferably, the linker comprises polyethylene glycol (PEG).

According to particular embodiments, the liposome further comprises one or more lipids selected from the group consisting of 1,2-dimyristoyl-sn-glycero-3-phosphocholine (DMPC), 1,2-dimyristoyl-sn-glycero-3-phosphoryl-3′-rac-glycerol (DMPG), and cholesterol.

According to particular embodiments, the liposome further comprises a buffer. Any suitable buffer known to those skilled in the art in view of the present disclosure can be used in the invention. In one embodiment, the liposome comprises a phosphate-buffered saline. According to particular embodiments, the buffer comprises histidine and sucrose.

An exemplary liposome used in the present invention comprises a Tau tetrapalmitoylated phosphopeptide (pTau Peptide T3, SEQ ID NO: 28) that is presented on the surface of the liposome via two palmitic acids at each terminus of the Tau peptide; A TLR-9 ligand comprising lipidated CpG (Adjuvant CpG7909 (CpG2006); SEQ ID NO: 18) incorporated into the liposome membrane via a cholesterol molecule that is covalently linked to the CpG via a PEG linker; a TLR-4 ligand (Monophosphoryl lipid A (e.g., 3D-(6-acyl) PHAD®)) incorporated into the membrane; an encapsulated helper T-cell epitope (PAN-DR binder T50; SEQ ID NO: 13); and 1,2-dimyristoyl-sn-glycero-3-phospho-choline (DMPC), 1,2-dimyristoyl-sn-glycero-3-[phospho-rac-(1-glycerol)] sodium salt (DMPG) and cholesterol as lipid components of the liposome.

Liposomes of the invention can be made using methods known in the art in view of the present disclosure. The optimal ratios of each component of the liposomes can be determined by techniques known to those skilled in the art in view of the present disclosure.

The liposomes can be administered by suitable means for prophylactic and/or therapeutic treatment. According to preferred embodiments, the liposomes are administered by subcutaneous or intramuscular injection. Intramuscular injection is most typically performed in the arm or leg muscles.

In one general aspect, the invention relates to pharmaceutical compositions comprising a therapeutically effective amount of liposome, together with a pharmaceutically acceptable excipient and/or carrier. Pharmaceutically acceptable excipients and/or carriers are well known in the art (see Remington's Pharmaceutical Science (15th ed.), Mack Publishing Company, Easton, Pa., 1980). The preferred formulation of the pharmaceutical composition depends on the intended mode of administration and therapeutic application. The compositions can include pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or non-toxic, non-therapeutic, non-immunogenic stabilizers, and the like. It will be understood that the characteristics of the carrier, excipient or diluent will depend on the route of administration for a particular application.

The target antigen for the vaccine is located in the brain, and the brain is separated from the circulation by a specialized cellular structure called the blood-brain barrier (BBB). The BBB restricts passage of substances from the circulation into the brain. This prevents the entry of toxins, microbes, etc. into the central nervous system. The BBB also has the potentially less desirable effect of preventing the efficient entry of immune mediators (such as antibodies) into the interstitial and cerebrospinal fluid that surrounds the brain.

Approximately 0.1% of antibodies that are present in the systemic circulation cross the BBB and enter the brain. This suggests that systemic titers induced by a vaccine targeting a CNS antigen must be at least 1000 times greater than the minimal effective titer to be efficacious in the brain. The minimum titers of antibodies in serum which are needed to trigger efficacy are not readily apparent. Additionally, not only the quantity but also the quality of the immune response (e.g., avidity) must be considered for a safe and effective immunotherapy targeting a CNS disorder, such as a neurodegenerative disease, disorder, or condition.

The avidity of an antibody can be measured by avidity index using methods known in the art in view of the present disclosure. The titers of antibodies against a particular antigen are measured at two different concentrations of the coated antigen: one is the saturated concentration, where all antibodies can bind to the antigen and another one is at a low concentration, where only antibodies with the highest binding capacity can bind to the antigen. As used herein, “avidity index” refers to the ratio of the levels of antibody titers measured at the low-and the high-density coating of the antigen. For example, avidity of antibodies against an antigen, such as ePHF or pTau, can be measured at different time points after an immunization or following different immunizations, to evaluate whether the avidity (as measured by the avidity index) increases over time. As used herein, antibodies with an “increased avidity” or “increased binding avidity” to an antigen refers to antibodies with an increased avidity index to the antigen over time during the course of a treatment or immunization. An increased avidity suggests a potential affinity maturation of the antibodies.

According to particular embodiments, the pharmaceutical compositions of the present invention therefore further comprise one or more suitable adjuvants to achieve the desired immune response in the subject. Suitable adjuvants can be administered before, after, or concurrent with administration of the liposome. Preferred adjuvants augment the intrinsic response to an immunogen without causing conformational changes in the immunogen that affect the qualitative form of the response. Examples of adjuvants are the aluminum salts (alum), such as aluminum hydroxide, aluminum phosphate, and aluminum sulfate. Such adjuvants can be used with or without other specific immunostimulating agents, such as MPLA Class (3 De-O-acylated monophosphoryl lipid A (MPL™), monophosphoryl hexa-acyl Lipid A 3-deacyl synthetic (3D-(6-acyl) PHAD®, PHAD™, PHAD®-504, 3D-PHAD®) lipid A), polymeric or monomeric amino acids, such as polyglutamic acid or polylysine. Such adjuvants can be used with or without other specific immunostimulating agents, such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′ dipalmitoyl-sn-glycero-3-hydroxyphosphoryloxy)-ethylamine (MTP-PE), N-acetylglucsaminyl-N-acetylmuramyl-L-Al-D-isoglu-L-Ala-dipalmitoxy propylamide (DTP-DPP) Theramide™), or other bacterial cell wall components. Oil-in-water emulsions include MF59 (see WO 90/14837), containing 5% Squalene, 0.5% Tween 80, and 0.5% Span 85 (optionally containing various amounts of MTP-PE) formulated into submicron particles using a microfluidizer; SAF, containing 10% Squalene, 0.4% Tween 80, 5% pluronic-blocked polymer L121, and thr-MDP, either microfluidized into a submicron emulsion or vortexed to generate a larger particle size emulsion; and the Ribi™ adjuvant system (RAS) (Ribi ImmunoChem, Hamilton, Mont.) 0.2% Tween 80, and one or more bacterial cell wall components selected from the group consisting of monophosphoryl lipid A (MPL™), trehalose dimycolate (TDM), and cell wall skeleton (CWS), preferably MPL™+CWS (Detox™). Other adjuvants include Complete Freund's Adjuvant (CFA), and cytokines, such as interleukins (IL-1, IL-2, and IL-12), macrophage colony stimulating factor (M-CSF), and tumor necrosis factor (TNF).

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject. In some embodiment of the invention, a pharmaceutical composition according to the invention, can be used in combination with a biologically active substance such as, for example, known compounds used in the medication of tauopathies and/or of amyloidosis, a group of diseases and disorders associated with amyloid or amyloid-like protein such as the amyloid β protein involved in Alzheimer's Disease. The other biologically active compound may include neutron-transmission enhancers, psychotherapeutic drugs, acetylcholine esterase inhibitors, calcium channel blockers, biogenic amines, benzodiazepine tranquillizers, acetylcholine synthesis, storage or release enhancers, acetylcholine postsynaptic receptor agonists, monoamine oxidase-A or -B inhibitors, N-methyl-0-aspartate glutamate receptor antagonists, non-steroidal anti-inflammatory drugs, antioxidants, and serotonergic receptor antagonists. In particular, the other biologically active compound may be selected from the group consisting of compounds against oxidative stress, anti-apoptotic compounds, metal chelators, inhibitors of DNA repair such as pirenzepin and metabolites, 3-amino-1-propanesulfonic acid (3APS), 1,3-propanedisulfonate (1,3PDS), secretase activators, and v-secretase inhibitors, tau proteins, neurotransmitter, β-sheet breakers, anti-inflammatory molecules, or cholinesterase inhibitors (ChEls) such as tacrine, rivastigmine, donepezil, and/or galantamine and other drugs and nutritive supplements, together with an therapeutic vaccine according to the invention and, optionally, a pharmaceutically acceptable carrier and/or a diluent and/or an excipient. In a further embodiment, the other biologically active compound may comprise niacin or memantine together with a liposome according to the invention and, optionally, a pharmaceutically acceptable carrier and/or a diluent and/or an excipient. In still another embodiment of the invention, other compounds comprises “atypical antipsychotics” such as, for example clozapine, ziprasidone, risperidone, aripiprazole or olanzapine for the treatment of positive and negative psychotic symptoms including hallucinations, delusions, thought disorders (manifested by marked incoherence, derailment, tangentiality), and bizarre or disorganized behavior, as well as anhedonia, flattened affect, apathy, and social withdrawal, together with a liposome of the invention. Other compounds that can be suitably used in combination with the pharmaceutical composition according to the invention are described, for example, in WO 2004/058258 (see especially pages 16 and 17) including therapeutic drug targets (page 36-39), alkanesulfonic acids and alkanolsulfuric acid (pages 39-51), cholinesterase inhibitors (pages 51-56), NMDA receptor antagonists (pages 56-58), estrogens (pages 58-59), non-steroidal anti-inflammatory drugs (pages 60-61), antioxidants (pages 61-62), peroxisome proliferators-activated receptors (PPAR) agonists (pages 63-67), cholesterol-lowering agents (pages 68-75); amyloid inhibitors (pages 75-77), amyloid formation inhibitors (pages 77-78), metal chelators (pages 78-79), anti-psychotics and anti-depressants (pages 80-82), nutritional supplements (pages 83-89) and compounds increasing the availability of biologically active substances in the brain (see pages 89-93) and prodrugs (pages 93 and 94), which document is incorporated herein by reference, but especially the compounds mentioned on the pages indicated above.

The timing of administrations can vary significantly from once a day, to once a year, to once a decade. A typical regimen consists of an immunization followed by booster injections at time intervals, such as 1 to 24-week intervals. Another regimen consists of an immunization followed by booster injections 1, 2, 4, 6, 8, 10 and 12 months later. Another regimen entails an injection every two months for life. Alternatively, booster injections can be on an irregular basis as indicated by monitoring of immune response.

It is readily appreciated by those skilled in the art that the regimen for the priming and boosting administrations can be adjusted based on the measured immune responses after the administrations. For example, the boosting compositions are generally administered weeks or months after administration of the priming composition, for example, about 1 week, or 2 weeks, or 3 weeks, or 4 weeks, or 8 weeks, or 16 weeks, or 20 weeks, or 24 weeks, or 28 weeks, or 32 weeks, or 36 weeks, or 40 weeks, or 44 weeks, or 48 weeks, or 52 weeks, or 56 weeks, or 60 weeks, or 64 weeks, or 68 weeks, or 72 weeks, or 76 weeks, or one to two years after administration of the priming composition.

According to particular aspects, one or more boosting immunizations can be administered. The antigens in the respective priming and boosting compositions, however many boosting compositions are employed, need not be identical, but should share antigenic determinants or be substantially similar to each other.

As known to those skilled in the art, immunogenicity, boostability and sustainability are important considerations for the effectiveness of a vaccine. It is discovered in the present invention that the administration of an effective amount of a liposome described herein is able to induce a potent antibody response against pTau in a patient in need thereof, such as a patient in need of treating an Alzheimer's Disease (e.g., mild to moderate Alzheimer's Disease or early Alzheimer's Disease) or mild cognitive impairment (MCI) due to Alzheimer's Disease. The antibody response is sustainable, e.g., lasting at least 6 weeks after the initial administration of the liposome. The antibody response is also boosted by one or more subsequent boosting administrations. As used herein, “boosted” in the context of an antibody response refers to the antibody response that is maintained or enhanced after a subsequent administration as measured at least two weeks after the administration of the subsequent administration. For example, an antibody response is “boosted” by a subsequent administration, if there is an increase of the antibody titer when measured 2 weeks after the subsequent administration as compared with the antibody titer before the subsequent administration.

Pharmaceutical compositions of the present invention can be formulated according to methods known in the art in view of the present disclosure. The optimal ratios of each component in the compositions can be determined by techniques known to those skilled in the art in view of the present disclosure.

In a preferred embodiment of the present invention, administration of a Tau peptide, via administration of a pharmaceutical composition according to an embodiment of the invention, induces an active immune response in the subject, such as an antibody response to the Tau peptide and to the pathological form of Tau, thereby facilitating the clearance of related Tau aggregates, slowing the progression of Tau-pathology related behavior and/or treating the underlying Tauopathy.

Tau is a human “self” protein. This means that, in principle, all lymphocytes bearing a receptor specific for tau should have been deleted during development (central tolerance) or rendered unresponsive by a peripheral tolerance mechanism. This problem has proved to be a significant roadblock to the development of vaccines against self or “altered self” proteins (e.g., tumor antigens). Generating high-quality antibodies against an antigen (self or infectious) requires the action of not only B lymphocytes, which produce the antibody, but also of CD4⁺ T “helper” lymphocytes. CD4⁺ T-cells provide critical survival and maturation signals to B lymphocytes, and CD4⁺ T-cell deficient animals are profoundly immunosuppressed. CD4⁺ T-cells are also subject to tolerance mechanisms, and an additional roadblock to generating strong anti-self (e.g., anti-tau) antibody responses is that tau-reactive CD4⁺ T-cells are also likely to be rare to non-existent in the human/animal repertoire.

In accordance with this aspect of the present invention, an immune response involves the development of a beneficial humoral (antibody mediated) response directed against the Tau peptide and a cellular (mediated by antigen-specific T cells or their secretion products) response directed against the T-cell epitope or the immunogenic carrier.

As used herein, a Tau-pathology related behavioral phenotype includes, without limitation, cognitive impairments, early personality change and disinhibition, apathy, abulia, mutism, apraxia, perseveration, stereotyped movements/behaviors, hyperorality, disorganization, inability to plan or organize sequential tasks, selfishness/callousness, antisocial traits, a lack of empathy, halting, agrammatic speech with frequent paraphasic errors but relatively preserved comprehension, impaired comprehension and word-finding deficits, slowly progressive gait instability, retropulsions, freezing, frequent falls, non-levodopa responsive axial rigidity, supranuclear gaze palsy, square wave jerks, slow vertical saccades, pseudobulbar palsy, limb apraxia, dystonia, cortical sensory loss, and tremor.

In carrying out the methods of the present invention, according to particular embodiments of the invention, it is preferable to select a subject having or at risk of having Alzheimer's Disease or other Tauopathy, a subject having Tau aggregates in the brain, or a subject exhibiting a tangle related behavioral phenotype prior to administering the immunogenic peptides or antibodies of the present invention. Subjects amenable to treatment include individuals at risk of disease but not showing symptoms, as well as patients presently showing symptoms. In the case of Alzheimer's Disease, virtually anyone is at risk of suffering from Alzheimer's Disease. Therefore, the present methods can be administered prophylactically to the general population without the need for any assessment of the risk of the subject patient. The present methods are especially useful for individuals who have a known genetic risk of Alzheimer's disease for the prevention or treatment of the disease. Such individuals include those having relatives who have experienced the disease, and those whose risk is determined by analysis of genetic or biochemical markers. In preferred embodiments, the subject is in need of a treatment of Alzheimer's Disease, preferably early Alzheimer's Disease, mild cognitive impairment (MCI) due to Alzheimer's Disease, mild Alzheimer's Disease, or mild to moderate Alzheimer's Disease. In another preferred embodiments, the subject is in need of a prevention of Alzheimer's Disease, preferably preclinical Alzheimer's Disease, early Alzheimer's Disease, mild cognitive impairment (MCI) due to Alzheimer's Disease, mild Alzheimer's Disease, or mild to moderate Alzheimer's Disease. The preclinical Alzheimer's Disease is a stage before early Alzheimer's Disease.

In asymptomatic patients, treatment can begin at any age (e.g., 10, 20, 30 years of age). Usually, however, it is not necessary to begin treatment until a patient reaches 40, 50, 60, or 70 years of age. Treatment typically entails multiple dosages over a period of time. Treatment can be monitored by assaying antibody, or activated T-cell or B-cell responses to the therapeutic agent over time. If the response decreases, a booster dosage is indicated.

In prophylactic applications, pharmaceutical compositions containing the Tau peptides are administered to a patient susceptible to, or otherwise at risk of, Alzheimer's Disease or other Tauopathy in an amount sufficient to eliminate or reduce the risk, lessen the severity, or delay the outset of the disease, including biochemical, histologic and/or behavioral symptoms of the disease, its complications and intermediate pathological phenotypes presented during development of the disease. In therapeutic applications, pharmaceutical compositions containing a Tau peptide are administered to a patient suspected of, or already suffering from, such a disease in an amount sufficient to cure, or at least partially arrest, the symptoms of the disease (biochemical, histologic and/or behavioral), including its complications and intermediate pathological phenotypes in development of the disease.

The composition can, if desired, be presented in a kit, pack or dispenser, which can contain one or more unit dosage forms containing the active ingredient. The kit, for example, can comprise metal or plastic foil, such as a blister pack. The kit, pack, or dispenser can be accompanied by instructions for administration.

### Embodiments

The invention provides also the following non-limiting embodiments.

Embodiment 1. A method of inducing an immune response, such as an antibody response against a phosphorylated Tau protein (pTau), in a human subject in need thereof, comprising administering to the subject an effective amount of a liposome comprising:
(1) a Tau phosphopeptide consisting of the amino acid sequence of SEQ ID NO:27 to SEQ ID NO:29 and SEQ ID NO:31 to SEQ ID NO:38 at an amount of 300 μg to 1800 μg per dose;
(2) a toll-like receptor 4 agonist comprising monophosphoryl lipid A;
(3) a helper T-cell epitope having an amino acid sequence selected from the group consisting of SEQ ID NO:13 to SEQ ID NO:17, SEQ ID NO:23 to SEQ ID NO:26, and SEQ ID NO: 39 to SEQ ID NO:44; and
(4) a CpG oligonucleotide having a nucleotide sequence selected from the group consisting of SEQ ID NO:18 to SEQ ID NO:22,
wherein:
the Tau phosphopeptide is presented on the surface of the liposome, and
the antibody response lasts at least 6 weeks after the initial administration of the effective amount of the liposome to the human subject.

Embodiment 2: The method of Embodiment 1, wherein the effective amount of the liposome comprises:
(1) the Tau phosphopeptide consisting of the amino acid sequence of SEQ ID NO: 28 at the amount of 300 μg to 1800 μg per dose;
(2) the toll-like receptor 4 agonist at an amount of 100 μg to 585 μg per dose;
(3) the helper T-cell epitope at an amount of 75 μg to 450 μg per dose; and
(4) the CpG oligonucleotide at an amount of 150 μg to 800 μg per dose.

Embodiment 3. The method of Embodiment 1 or 2, wherein the effective amount of the liposome comprises 300 μg, 900 μg or 1800 μg per dose of the Tau phosphopeptide.

Embodiment 3a. The method of Embodiment 1 or 2, wherein the effective amount of the liposome comprises 300 μg per dose of the Tau phosphopeptide.

Embodiment 3b. The method of Embodiment 1 or 2, wherein the effective amount of the liposome comprises 900 μg per dose of the Tau phosphopeptide.

Embodiment 3c. The method of Embodiment 1 or 2, wherein the effective amount of the liposome comprises 1800 μg per dose of the Tau phosphopeptide.

Embodiment 4. The method of any one of Embodiments 1-3c, wherein the effective amount of the liposome is administered subcutaneously.

Embodiment 5. The method of any one of Embodiments 1-3c, wherein the effective amount of the liposome is administered intramuscularly.

Embodiment 6. The method of any one of Embodiments 1-5, wherein the CpG oligonucleotide has one or more phosphorothioate internucleotide linkages, and the CpG oligonucleotide is covalently linked to at least one lipophilic group, optionally via a PEG linker.

Embodiment 7. The method of any one of Embodiments 1-6, wherein the Tau phosphopeptide consists of the amino acid sequence of SEQ ID NO:28, the toll-like receptor 4 agonist comprises monophosphoryl hexa-acyl Lipid A, 3-deacyl, the helper T-cell epitope comprises the amino acid sequence of SEQ ID NO: 39, the CpG oligonucleotide comprises the nucleotide sequence of SEQ ID NO: 18, and the liposome further comprises at least one lipid selected from the group consisting of 1,2-dimyristoyl-sn-glycero-3-phosphocholine (DMPC), 1,2-dimyristoyl-sn-glycero-3-phosphoryl-3′-rac-glycerol (DMPG), and cholesterol.

Embodiment 8. The method of any one of Embodiments 1-7, wherein the antibody response has conformation specificity against pathological Tau protein, which increases over time after the initial administration of the effective amount of the liposome to the human subject, preferably wherein the antibody response comprises a specific IgG antibody response directed against the pTau, preferably the specific IgG antibody response has an anti-pTau IgG titer at least 50, 60, 70, 80, 90, 100 or more times higher than that of a placebo control.

Embodiment 9. The method of any one of Embodiments 1-8, wherein the antibody response induces a class switch of a specific IgM antibody response to a specific IgG antibody response directed against the pTau, with indication for memory building.

Embodiment 10. The method of any one of Embodiments 1-9, wherein the antibody response comprises an IgG immune response that preferentially recognizes the pTau over non-phosphorylated Tau protein, preferably the ratio of the anti-pTau IgG titer to the anti-Tau IgG titer is at least 5, 10, 15, 20, 25, 30, 35, 40, 45, 50, 55, 60, 65 or 70.

Embodiment 10a. The method of any one of Embodiments 1-10, wherein the IgG immune response against pTau is maintained over time.

Embodiment 10b. The method of any one of Embodiments 1-10a, wherein the IgG immune response against non-phosphorylated Tau protein becomes lower over time.

Embodiment 11. The method of any one of Embodiments 1-10b, wherein the antibody response comprises an IgG immune response against an enriched Paired Helical Filament (ePHF).

Embodiment 12. The method of Embodiment 11, wherein the IgG immune response has an anti-ePHF IgG titer at least 2, 3, 4, 5, 6, 7, 8, 9, 10, 11, 12, 13, 14, 15, 16, 17, 18, 19, 20 or more times higher than that of a placebo control.

Embodiment 12a. The method of Embodiments 11 or 12, wherein the IgG immune response matures toward a stronger preference for binding to ePHF while concomitantly lowering antibody titers towards the non-phosphorylated Tau.

Embodiment 12b. The method of Embodiments 11 or 12, wherein the IgG immune response has a higher IgG titer towards ePHF than an IgG titer towards non-phosphorylated Tau.

Embodiment 13. The method of Embodiments 11, 12, 12a or 12b, wherein the anti-ePHF IgG has an increased binding avidity to the pathological ePHF Tau for at least 6 weeks, 8 weeks, 10 weeks, 12 weeks, 14 weeks, 16 weeks, 18 weeks, 20 weeks, 22 weeks, 24 weeks or longer after the initial administration of the effective amount of the liposome, preferably the anti-ePHF IgG has an avidity index of at least 0.3, 0.4, 0.5, 0.6, or 0.7.

Embodiment 14. The method of any one of Embodiments 1-13, further comprising administering to the subject a second dose of the effective amount of liposome 4 to 12 weeks, such as 8 weeks, after the initial administration of the effective amount of liposome.

Embodiment 14a. The method of Embodiment 14, wherein the effective amount of liposome comprises 300 μg per dose of the Tau phosphopeptide for each of the initial administration and the second dose, and the second dose is administered to the subject 8 weeks after the initial administration.

Embodiment 14b. The method of Embodiment 14, wherein the effective amount of liposome comprises 900 μg per dose of the Tau phosphopeptide for each of the initial administration and the second dose, and the second dose is administered to the subject 8 weeks after the initial administration.

Embodiment 14c. The method of Embodiment 14, wherein the effective amount of liposome comprises 1800 μg per dose of the Tau phosphopeptide for each of the initial administration and the second dose, and the second dose is administered to the subject 8 weeks after the initial administration.

Embodiment 15. The method of any one of Embodiments 14-14c, wherein the antibody response comprising the IgG immune response against pTau is boosted after the administration of the second dose of the effective amount of liposome, preferably the antibody response is increased at least 10%, 20%, 30%, 40%, 50%, 60%, 70%, 80%, 90%, 100% or more as measured at least 2 weeks after the administration of the second dose of the effective amount of liposome.

Embodiment 15a. The method of Embodiment 15, wherein the anti-ePHF IgG response is boosted after the administration of the second dose of the effective amount of liposome as measured at least 2 weeks after the administration of the second dose of the effective amount of liposome.

Embodiment 16. The method of any one of Embodiments 14-15a, further comprising administering to the subject a third dose of the effective amount of liposome 20 to 28 weeks, such as 24 weeks, after the initial administration of the effective amount of liposome.

Embodiment 16a. The method of Embodiment 16, wherein the effective amount of liposome comprises 300 μg per dose of the Tau phosphopeptide for each of the initial administration, the second dose and the third dose, and the second dose and the third dose are respectively administered to the subject 8 weeks and 24 weeks after the initial administration.

Embodiment 16b. The method of Embodiment 16, wherein the effective amount of liposome comprises 900 μg per dose of the Tau phosphopeptide for each of the initial administration, the second dose and the third dose, and the second dose and the third dose are respectively administered to the subject 8 weeks and 24 weeks after the initial administration.

Embodiment 16c. The method of Embodiment 16, wherein the effective amount of liposome comprises 1800 μg per dose of the Tau phosphopeptide for each of the initial administration, the second dose and the third dose, and the second dose and the third dose are respectively administered to the subject 8 weeks and 24 weeks after the initial administration.

Embodiment 17. The method of any one of Embodiments 16-16c, wherein the antibody response comprising the IgG immune response against pTau is boosted after the administration of the third dose of the effective amount of the liposome, preferably the antibody response is increased at least 10%, 20%, 30%, 40%, 50%, 60%, 70%, 80%, 90%, 100% or more as measured at least 2 weeks after the administration of the third dose of the effective amount of liposome.

Embodiment 17a. The method of Embodiment 17, wherein the anti-ePHF IgG response is boosted after the administration of the third dose of the effective amount of liposome as measured at least 2 weeks after the administration of the second dose of the effective amount of liposome.

Embodiment 18. The method of any one of Embodiments 16-17a, further comprising administering to the subject a fourth dose of the effective amount of liposome 44 to 52 weeks, such as 48 weeks, after the initial administration of the effective amount of liposome.

Embodiment 18a. The method of Embodiment 18, wherein the effective amount of liposome comprises 300 μg per dose of the Tau phosphopeptide for each of the initial administration, the second, third and fourth doses, and the second, third and fourth doses are respectively administered to the subject 8 weeks, 24 weeks and 48 weeks after the initial administration.

Embodiment 18b. The method of Embodiment 18, wherein the effective amount of liposome comprises 900 μg per dose of the Tau phosphopeptide for each of the initial administration, the second, third and fourth doses, and the second, third and fourth doses are respectively administered to the subject 8 weeks, 24 weeks and 48 weeks after the initial administration.

Embodiment 18c. The method of Embodiment 18, wherein the effective amount of liposome comprises 1800 μg per dose of the Tau phosphopeptide for each of the initial administration, the second, third and fourth doses, and the second, third and fourth doses are respectively administered to the subject 8 weeks, 24 weeks and 48 weeks after the initial administration.

Embodiment 19. The method of Embodiment 18, wherein the antibody response comprising the IgG immune response against pTau is boosted after the administration of the fourth dose of the effective amount of liposome, preferably the antibody response is increased at least 10%, 20%, 30%, 40%, 50%, 60%, 70%, 80%, 90%, 100% or more as measured at least 2 weeks by the administration of the fourth dose of the effective amount of liposome.

Embodiment 19a. The method of Embodiment 19, wherein the anti-ePHF IgG response is boosted after the administration of the fourth dose of the effective amount of liposome as measured at least 2 weeks after the administration of the second dose of the effective amount of liposome.

Embodiment 20. A method of inducing a sustained immune response against a phosphorylated Tau protein (pTau) in a human subject in need thereof, comprising:
i. intramuscularly administering to the subject a primer vaccine comprising an effective amount of a liposome; and
ii. intramuscularly administering to the subject a first booster vaccine comprising the effective amount of the liposome 6-10 weeks after the administration of the primer vaccine,
   wherein:
the sustained immune response lasts at least about 20 weeks after the administration of the primer vaccine;
the liposome comprises:
   (1) a Tau phosphopeptide consisting of the amino acid sequence of SEQ ID NO: 28, and the Tau phosphopeptide is presented on the surface of the liposome;
   (2) a toll-like receptor 4 agonist comprising monophosphoryl lipid A;
   (3) a helper T-cell epitope having an amino acid sequence selected from the group consisting of SEQ ID NOs:23, 24, 25, and 26; and
   (4) a CpG oligonucleotide having a nucleotide sequence selected from the group consisting of SEQ ID NO:18 to SEQ ID NO:22; and
the effective amount of the liposome comprises:
   (1) the Tau phosphopeptide at an amount of 300 μg to 1800 μg per dose;
   (2) the toll-like receptor 4 agonist at an amount of 100 μg to 585 μg per dose;
   (3) the helper T-cell epitope at an amount of 75 μg to 550 μg per dose, such as 75 μg to 450 μg, 80 μg to 540 μg, 82.5 μg to 535 μg, 85 μg to 530 μg, 87.5 μg to 525 μg, or 90 μg to 520 μg per dose; and
   (4) the CpG oligonucleotide at an amount of 100 μg to 1000 μg, such as 150-800 μg, 125 μg to 950 μg, 150 μg to 900 μg, or 150 μg to 850 μg per dose.

Embodiment 20a. The method of Embodiment 20, wherein the helper T-cell epitope has an amino acid sequence selected from the group consisting of SEQ ID NOs:39, 40, 41, 42, and 43.

Embodiment 20b. The method of Embodiment 20, wherein the helper T-cell epitope has an amino acid sequence selected from the group consisting of SEQ ID NOs: 13, 14, 15, 16, 17, and 44.

Embodiment 20c. The method of any one of Embodiments 20-20b, wherein the lipidated CpG oligonucleotide has the nucleotide sequence of SEQ ID NO:18 and the CpG oligonucleotide is covalently linked to at least one cholesterol via a linker.

Embodiment 20d. The method of any one of Embodiments 20-20c, wherein the first booster vaccine is administered 8 weeks after the administration of the primer vaccine, and the sustained immune response lasts at least about 24 weeks after the administration of the primer vaccine.

Embodiment 20e. The method of any one of Embodiments 20-20d, further comprising intramuscularly administering to the subject a second booster vaccine comprising the effective amount of the liposome 22-26 weeks after the administration of the primer vaccine, and the sustained immune response lasts at least about 36 weeks after the administration of the primer vaccine.

Embodiment 20f. The method of Embodiment 20e, wherein the second booster vaccine is administered 24 weeks after the administration of the primer vaccine, and the sustained immune response lasts at least about 48 weeks after the administration of the primer vaccine.

Embodiment 20g. The method of Embodiment 20e or 20f, further comprising intramuscularly administering to the subject a third booster vaccine comprising the effective amount of the liposome 45-50 weeks after the administration of the primer vaccine, and the sustained immune response lasts at least about 60 weeks after the administration of the primer vaccine.

Embodiment 20h. The method of Embodiment 20g, wherein the third booster vaccine is administered 48 weeks after the administration of the primer vaccine, and the sustained immune response lasts at least about 72 weeks after the administration of the primer vaccine.

Embodiment 20i. The method of any one of Embodiments 20-20h, wherein the effective amount of the liposome comprises 300 μg per dose of the Tau phosphopeptide.

Embodiment 20j. The method of any one of Embodiments 20-20h, wherein the effective amount of the liposome comprises 900 μg per dose of the Tau phosphopeptide.

Embodiment 20k. The method of any one of Embodiments 20-20h, wherein the effective amount of the liposome comprises 1800 μg per dose of the Tau phosphopeptide.

Embodiment 20k1. The method of any one of Embodiments 20i-20k, wherein the effective amount of the liposome comprises 80 μg to 540 μg per dose of the helper T-cell epitope, and 125 μg to 950 μg per dose of the CpG oligonucleotide.

Embodiment 20k2. The method of any one of Embodiments 20i-20k, wherein the effective amount of the liposome comprises 82.5 μg to 535 μg per dose of the helper T-cell epitope, and 125 μg to 950 μg per dose of the CpG oligonucleotide.

Embodiment 20k3. The method of any one of Embodiments 20i-20k, wherein the effective amount of the liposome comprises 87.5 μg to 525 μg per dose of the helper T-cell epitope, and 150 μg to 900 μg per dose of the CpG oligonucleotide.

Embodiment 20k4. The method of any one of Embodiments 20i-20k, wherein the effective amount of the liposome comprises 85 μg to 530 μg per dose of the helper T-cell epitope, and 150 μg to 900 μg per dose of the CpG oligonucleotide.

Embodiment 20l. The method of any one of Embodiments 20-20k4, wherein the sustained immune response comprises an IgG immune response that preferentially recognizes the pTau over non-phosphorylated Tau protein, preferably the ratio of the anti-pTau IgG titer to the anti-Tau IgG titer is at least 5, 10, 15, 20, 25, 30, 35, 40, 45, 50, 55, 60, 65 or 70.

Embodiment 20m. The method of any one of Embodiments 20-20l, wherein the sustained immune response comprises an IgG immune response against enriched Paired Helical Filament (ePHF) having an anti-ePHF IgG titer at least 2, 3, 4, 5, 6, 7, 8, 9, 10, 11, 12, 13, 14, 15, 16, 17, 18, 19, 20 or more times higher than that of a placebo control.

Embodiment 21. The method of any one of Embodiments 1-20m, wherein the subject is in need of clearance of aggregates of Tau.

Embodiment 22. The method of any one of Embodiments 1-21, wherein the subject is in need of a treatment of Alzheimer's Disease, such as preclinical Alzheimer's Disease, mild to moderate Alzheimer's Disease or early Alzheimer's Disease, mild cognitive impairment (MCI) due to Alzheimer's Disease.

Embodiment 23. The method of any one of Embodiments 1-21, wherein the subject is in need of a prevention of Alzheimer's Disease, such as preclinical Alzheimer's Disease, mild to moderate Alzheimer's Disease or early Alzheimer's Disease, mild cognitive impairment (MCI) due to Alzheimer's Disease.

Embodiment 24. The method of any one of Embodiments 1-23, wherein the immune response comprises an anti-phosphorylated Tau antibody that binds specifically to an epitope comprising phosphorylated Ser396.

Embodiment 25. The method of Embodiment 24, wherein the epitope further comprises phosphorylated Ser404.

### EXAMPLES

The following examples of the invention are to further illustrate the nature of the invention. It should be understood that the following examples do not limit the invention and that the scope of the invention is to be determined by the appended claims.

The experimental methods used in the following examples, unless otherwise indicated, are all ordinary methods. The reagents used in the following embodiments, unless otherwise indicated, are all purchased from ordinary reagent suppliers.

In all following examples, ACI-35.030 is a liposome formulation according to

embodiments of the invention that contains a phosphorylated Tau peptide having the amino acid sequence of SEQ ID NO: 28, MPLA (3D-(6-acyl) PHAD®), DMPC, DMPG, cholesterol, a helper T-cell epitope of SEQ ID NO:13, a CpG2006 oligonucleotide covalently linked to a cholesterol group via a PEG linker, and a buffer, and ACI-35 is a liposome formulation according to embodiments of the invention that contains a phosphorylated Tau peptide having the amino acid sequence of SEQ ID NO: 28, MPLA, DMPC, DMPG, cholesterol, and a buffer.

### Example 1 Clinical Study on the Safety and Efficacy of ACI-35.030 in Humans

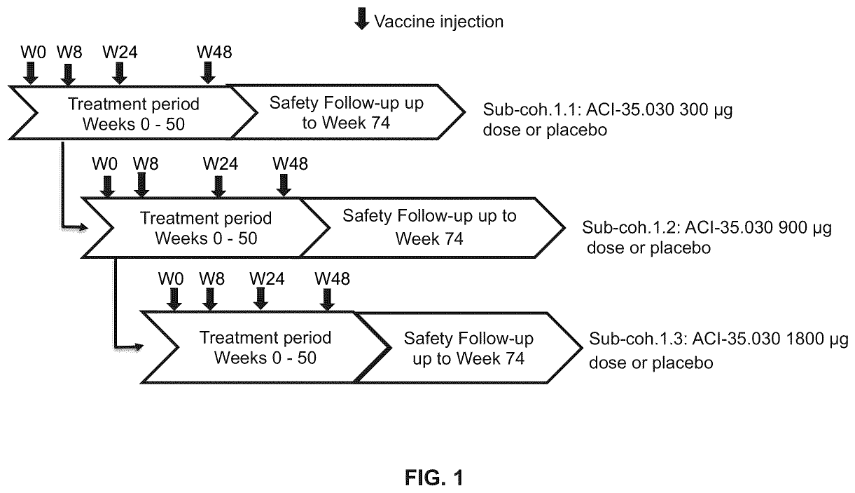

The safety, tolerability and immunogenicity of ACI-35.030 vaccine (ACI-35.030) is evaluated in a clinical Phase 1b/2a multicenter, double blind, randomized, placebo-controlled study conducted in patients with early AD (mild cognitive impairment (MCI) due to AD or mild AD) in Europe (ACI-35-1802 study). See also, study number NCT04445831 in clinicaltrials.gov. A summary of the design of the study is shown in FIG. 1.

Objective: To assess ACI-35.030 at up to 3 dosages in patients with early AD (e.g., mild cognitive impairment (MCI) due to AD or mild AD), for the safety and tolerability in patients with early AD, and for the induction of an antibody response against the abnormal form of Tau protein, including induction of anti-phospho-Tau antibodies (e.g., that bind to anti-pTau and ePHF Tau) in serum, in a time frame of 74 weeks.

Secondary objectives: To further assess the immunogenicity of study vaccines by assessing, e.g., the induction of IgG titers against Tau and of IgM titers against pTau and Tau in serum; and to assess the avidity of antibodies elicited by immunization, in a time frame of 74 weeks.

Exploratory objectives: To explore the effect of study vaccines on putative biomarkers of the progression of AD, e.g., blood and/or CSF concentrations of total Tau and pTau proteins; to explore the effect of study vaccines on the activation of T-cell in blood; to explore the effect of study vaccines on blood inflammatory cytokines (e.g., IL-1β, IL-2, IL-6, IL-8, IL-10, IFN-γ, and TNF-α); to explore the effect of study vaccines on behavior, cognitive and functional performance, each in a time frame of 74 weeks.

Methods: Each of 3 sub-cohorts consists of patients receiving placebo or different dosages of ACI-35.030, referred to by the amount of pTau Peptide T3 in the composition (300 μg, 900 μg or 1800 μg of tetrapalmitoylated phosphopeptide pTau Peptide T3, SEQ ID NO: 28) spread over 48 weeks (dose administrations at weeks 0, 8, 24 and 48), followed by a 24-week follow-up period.

Forty-one patients were randomized into the 3 sub-cohorts to receive either ACI-35.030 or placebo in each sub-cohort (active/placebo ratio 3:1). Doses were administered intramuscularly.

A safety assessment was/is performed immediately after each dosing and 48 to 72 hours thereafter by telephone call for all study patients. In each sub-cohort, the first dosing of the first 4 patients was/is performed once the safety assessment at 48 to 72 hours of the previous patient has been performed to confirm there is no clinically relevant safety issue related to study vaccine, according to the site principal investigator.

All treated patients have a follow-up period of 24 weeks after the end of the treatment period. During this period, patients are asked to attend a first follow-up visit 19 weeks after the last administration and a last visit at the end of the follow-up period (26 weeks after the last administration). Patients' safety is monitored throughout the study with regular review of safety data by an independent Data and Safety Monitoring Board (DSMB).

Interim analyses were/are carried out as follows:

The first interim analyses were conducted in all sub-cohorts conducted in cohort 1 once all subjects in their respective sub-cohorts have completed visit 4 (Week 10), i.e., 2 weeks after the second injection. The objective was to review safety, tolerability, and immunogenicity data for ACI-35.030 up to this time point.

The second interim analyses were/are conducted in sub-cohorts 1.1, 1.2, and 1.3 once all subjects in their respective sub-cohort have completed visit 6 (Week 26), i.e., 2 weeks after the third injection. The objective is to decide to expand potentially cohort 1 in order to collect additional safety/tolerability data at the dose presenting the most favorable profile in terms of immunogenicity, safety and tolerability.

The third interim analysis was performed at the end of the treatment period (i.e., 2 weeks after the 4th injection). The objective is to review the safety/tolerability and immunogenicity data up to this time point, including data from patients of sub-cohort expansion if applicable. Biomarker results can be included as supportive exploratory data. The results are compared with those subsequently obtained for other cohorts in order to select, among all study cohorts, the best strategy for further clinical development.

The fourth interim analysis was/is performed at the end of the follow-up period, i.e., once all cohort 1 patients have completed visit 11 (Week 74). The objective is the same as in the fourth interim analysis and the results are subsequently compared across all cohorts.

The study population is 50-75 years of age (male and female) with a diagnosis of mild AD or MCI due to AD according to NIA-AA criteria.

Inclusion criteria are as follows:
1. Male or female with age from 50 and up to 75 years old inclusive.
2. Mild Cognitive Impairment (MCI) due to AD or mild AD according to NIA-AA criteria and a Clinical Dementia Rating scale (CDR) global score of 0.5 or 1.
3. Mini mental state examination (MMSE) score of 22 or above.
4. Levels of CSF amyloid beta 42 (Aβ42) and phosphorylated Tau at screening consistent with NIA-AA 2018 criteria for AD pathology. In borderline cases for CSF Aß42 levels, other results may be considered to help determine amyloid positivity e.g., the Aß42/Aß40 ratio and, on a case by case basis, a history of positive amyloid PET scan or positive CSF Aß42 level. Results from CSF sampling performed within 3 months prior to screening are acceptable on a case by case basis provided that they are consistent with the presence of amyloid pathology and that the corresponding CSF sample can be used in the study for testing.
5. Patients either not taking any marketed treatment for AD or receiving a stable dose of an acetylcholinesterase inhibitor and/or memantine for at least 3 months prior to baseline.
6. Patients cared for by a reliable informant or caregiver to assure compliance, assist with clinical assessments and report safety issues.
7. Women must be post-menopausal for at least one year and/or surgically sterilized. Women of childbearing potential or not post-menopausal must have a negative pregnancy test at screening and be willing to use highly effective methods of contraception from the screening visit until the end of their participation. Urine pregnancy re-test will be performed throughout the treatment period to determine if the subject can continue receiving the study vaccine. Male patients with partners of childbearing potential must be willing to use appropriate contraceptive measures during the study.
8. Patient who in the opinion of the investigator is able to understand and provide written informed consent.
9. Patients and informant or caregiver must be fluent in one of the languages of the study and able to comply with all study procedures, including lumbar punctures.

Exclusion criteria are as follows:
1. Participation in previous clinical trials for AD and/or for neurological disorders using active immunization unless there is documented evidence that the patient was treated with placebo only and the placebo formulation is not expected to induce any specific immune response.
2. Participation in previous clinical trials for AD and/or for neurological disorders using any passive immunization within the past 6 months (or 5 half-lives of the investigational antibody, whichever is longer) months prior to screening unless there is documented evidence that the subject was treated with placebo only and the placebo is not expected to induce any specific immune response.
3. Participation in previous clinical trials for AD and/or for neurological disorders using any small molecule drug including BACE-1 inhibitors within the past 3 months prior to screening.
4. Concomitant participation to any other clinical trial using experimental or approved medications or therapies.
5. Presence of positive anti-nuclear antibody (ANA) titers at a dilution of at least 1/160 in patients without clinical symptoms of auto-immune disease.
6. Current or past history of auto-immune disease, or clinical symptoms consistent with the presence of auto-immune disease.
7. Immune suppression including but not limited to the use of immunosuppressant drugs or systemic steroids unless they have been prescribed transiently more than 3 months prior to screening.
8. History of severe allergic reaction (e.g., anaphylaxis) including but not limited to severe allergic reaction to previous vaccines and/or medications.
9. Prior history of clinically significant hypoglycemic episodes.
10. Drug or alcohol abuse or dependence currently met or within the past five years according to Diagnostic and Statistical Manual of Mental Disorders-V (DSM-V) criteria.
11. Any clinically significant medical condition likely to interfere with the evaluation of safety and tolerability of the study treatment and/or the adherence to the full study visit schedule.
12. Any clinically significant medical condition likely to impact on the immune system and/or expected to potentially impair the immunization potential of the study vaccine in patients (e.g., any history of acquired or innate immunodepressive disorder).
13. Use of hydralazine, procainamide, quinidine, isoniazide, TNF-inhibitors, minocycline within the last 12 months prior to screening.
14. Use of diltiazem unless on a stable dose for at least 3 months prior to screening.
15. Significant risk of suicide defined, using the Columbia-Suicide Severity Rating Scale, as the subject answering: “yes” to suicidal ideation questions 4 or 5 or answering: “yes” to suicidal behavior within the past 12 months.
16. Concomitant psychiatric or neurologic disorder other than those considered to be related to AD (e.g., head injury with loss of consciousness, symptomatic stroke, Parkinson's disease, severe carotid occlusive disease, TIAs).
17. History or presence of uncontrolled seizures. If history of seizures, they must be well controlled with no occurrence of seizures within 2 years prior to baseline. The use of anti-epileptic medications is permitted if at stable dose for at least 3 months prior to screening.
18. History of meningoencephalitis within the past 10 years prior to screening.
19. Patients with a history of hemorrhagic and/or non-hemorrhagic stroke.
20. Presence or history of peripheral neuropathy.
21. History of inflammatory neurological disorders with potential for CNS involvement.
22. Screening MRI scan showing structural evidence of alternative pathology not consistent with AD which could cause the patient's symptoms. Evidence of space occupying lesions other than benign meningioma of less than 1 cm diameter, more than two lacunar infarcts or one single infarct larger than 1 cm in diameter or any single area of superficial siderosis or evidence of a prior macro-hemorrhage≥10 mm. Microbleeds on T2*MRI are allowed up to a maximum of 10, regardless of the location.
23. MRI examination cannot be done for any reason, including but not limited to metal implants contraindicated for MRI studies and/or severe claustrophobia.
24. Significant hearing or visual impairment or other issues judged relevant by the investigator preventing to comply with the protocol and to perform the outcome measures.
25. Clinically significant infections or major surgical operation within 3 months prior to screening. Planned surgery anticipated to occur during participation in the study must be reviewed and approved by the medical monitor at screening.
26. Any vaccine received within the past 2 weeks before baseline, including influenza vaccine.
27. Clinically significant arrhythmias or other clinically significant abnormalities on ECG at screening.
28. Myocardial infarction within one year prior to baseline, unstable angina pectoris, or significant coronary artery disease.
29. Patients with a history of cancer within the past 5 years other than treated squamous cell carcinoma, basal cell carcinoma and melanoma in situ, or in-situ prostate cancer or in-situ breast cancer which have been fully removed and are considered cured.
30. In the opinion of the site investigator, clinically significant deviations from normal values for hematologic parameters, liver function tests, and other biochemical measures, that are judged to be clinically significant.
31. Female subjects being pregnant as confirmed by serum testing at screening or planning to be pregnant or lactating.
32. Patient receiving any anticoagulant drug or antiplatelet drug, except aspirin at doses lower than 100 mg daily (in order to avoid risk of bleeding during scheduled or unscheduled lumbar puncture).
33. Patients receiving antipsychotic drugs unless on stable low doses for the treatment of insomnia.
34. Patients who have donated blood or blood products during the 30 days prior to screening or who plan to donate blood while participating in the study.
35. Positive VDRL (Venereal Disease Research Laboratory) consistent with active syphilis at screening.
36. Patients with a positive HIV test at screening.
37. Patients with active hepatitis B and/or C as measured by testing at screening.
38. Patients with creatinine greater than 1.5× upper limit of normal, abnormal thyroid function tests or clinically significant reduction in serum B12 or folate levels (note: all oral doses of thyroid replacement agents, B12 or folate have to be stable for at least 3 months prior to screening).

### Patient Demographics

The study is ongoing. The patient demographics for sub-cohorts 1.1 and 1.2 (as of the cut-off date of the end of September 2021) are summarized in Table 1.

TABLE 1

| | Sub-cohort 1.1 | Sub-cohort 1.2 |
|---|---|---|
| Age (Years) {Range} | 65.3 {61-75} | 65 {51-71} |
| Sex (F/M) | 5/3 | 4/4 |
| MMSE (Mean) {Range} | 26.3 {22/29} | 26.4 {24/29} |

| | |
|---|---|
| Ethnicity | All patients white non-Hispanic |

### Results/Conclusions

The following primary endpoints were/are assessed:

Safety and tolerability—adverse events, immediate and delayed reactogenicity (e.g., anaphylaxis, local and systemic reactogenicity, including pain, redness, immune-complex disease, swelling, fever); global assessment of tolerability; suicidal ideation (C-SSRS); behavior (NPI); cognitive and functional assessments (RBANS, CDR-SB) to assess safety; vital signs; MRI imaging; electrocardiogram; routine hematology and biochemistry evaluation in blood and urine; evaluation of autoimmune antibodies including anti-DNA antibodies in blood; inflammatory markers in blood and CSF.

Immune response—anti-pTau IgG titers in serum (geometric mean, change from baseline, responder rate, peak and area under the curve).

The following secondary endpoints were/are assessed:

Immune response—anti-Tau IgG, anti-pTau IgM, anti-ePHF IgG and anti-Tau IgM titers in serum (geometric mean, change from baseline, responder rate, peak and area under the curve), determination of IgG response profile by avidity testing.

The following exploratory endpoints were/are assessed:

Change from baseline of biomarkers titers in blood and/or CSF (e.g., total Tau and pTau proteins), change from baseline in T-cell activation level in blood, change from baseline of inflammatory cytokine (e.g., IL-1B, IL-2, IL-6, IL-8, IL-10, IFN-γ, and TNF-α) titers in blood, change from baseline in suicidal ideation (C-SSRS), behavior (NPI), cognitive and functional performance (RBANS, CDR-SB) scores.

The study is ongoing. Three (3) sub-cohorts have received ACI-35.030 at the dosage level of 300 μg, 900 μg or 1800 μg of tetrapalmitoylated phosphopeptide pTau Peptide T3 (SEQ ID NO: 28), and placebo as in Table 2.

TABLE 2
Design of the clinical study.

| | | | Dose (T3 | | Number of | |
|---|---|---|---|---|---|---|
| | Sub- | Study | peptide | | early AD | Route of |
| Cohort | cohort | treatment | μg) | | subjects # | administration |
| 1 | 1.1 | ACI-35.030 | 300 | μg | 6 | Intramuscular |
| | | Placebo | 0 | μg | 2 | (i.m.) |
| | | (saline) | | | | |
| | 1.2 | ACI-35.030 | 900 | μg | 18 or 19 | |
| | | Placebo | 0 | μg | 6 or 7 | |
| | | (saline) | | | | |
| | 1.3 | ACI-35.030 | 1800 | μg | 6 | |
| | | Placebo | 0 | μg | 2 | |
| | | (saline) | | | | |

Dose administered 4 times at following intervals: Week 0, 8, 24, and 48 for each sub-cohort.
Blood samples for antibody determination withdrawn at following timepoints: Screening, Weeks 0 (pre-dose), 2, 8, 10, 15*, 20*, 24, 26, 31*, 36, 42*, 48, 50, 67 and 74.
(*): additional timepoints added for certain subjects;
# Includes MCI due to AD as well as mild AD subjects
Note:
There are 25 subjects in sub-cohort 1.2 treated with ACI-35.030 or placebo. The study is still blinded.

The interim safety and tolerability results as of the cut-off date of Jun. 20, 2022 indicated that no safety or tolerability issues were identified for ACI-35.030. Among sub-cohorts 1.1, 1.2 and 1.3 in cohort 1, there were no withdrawals due to adverse events. Additionally, the patients examined to-date exhibited no CNS inflammation or other significant changes reported on MRI.

An increased anti-pTau-specific IgG titer 2 weeks after administration of ACI-35.030 relative to baseline was observed in the serum of 100% of the subjects after the first administration of 300 μg, 900 μg or 1800 μg of tetrapalmitoylated phosphopeptide pTau Peptide T3 (SEQ ID NO: 28). This anti-pTau IgG response showed a preference for pTau over non-pTau peptide in all actively treated early AD subjects, and the antibody response was boosted by the additional administrations of ACI-35.030, as shown by increased anti-pTau-specific IgG titers and/or increased anti-ePHF IgG titers measured 2 weeks after the additional administrations of ACI-35.030. No antibody response was observed in subjects receiving the placebo, except one single limited elevation of IgM and IgG anti-pTau titers after the study treatment period at week 67 in one subject in sub-cohort 1.2 receiving placebo, for which the responses were close to the threshold set for defining a responder.

### Anti-pTau IgG Response of ACI-35.030 in Humans

Specific IgG antibody responses directed against the phosphorylated Tau peptide (pTau) induced by ACI-35.030 vaccine in the three sub-cohorts of Table 2 were measured by MSD.

Table 3 shows the anti-pTau IgG titers and responder rate (ITT population) following immunization with either ACI-35.030 at the dosage level of 300 μg of tetrapalmitoylated phosphopeptide pTau Peptide T3 (ACI-35.030 300 μg) or placebo in sub-cohort 1.1.

TABLE 3

| | Sub-cohort 1.1 | | | |
|---|---|---|---|---|
| | ACI-35.030 300 μg | | | |
| Treatment | (n = 6) | | Placebo (n = 2) | |
| groups | Antibody | | Antibody | |
| (number of | titers | Responder | titers | Responder |
| subjects) | (AU/mL) | rate^{♦} | (AU/mL) | rate^{♦} |
| Baseline † | | | | |
| Geom. Mean | 2048 (2.05) | — | 744 (1.56) | — |
| (Geom. Std) | | | | |
| Min; max | 939; 7980 | | 544; 1018 | |
| 95% CI | 1155; 3632 | | 402; 1376 | |
| Week 2 | | | | |
| Geom. Mean | 80292 (2.59) | 6/6 | 732 (1.29) | 0/2 |
| (Geom. Std) | | | | |
| Min; max | 27300; 336000 | | 613; 875 | |
| 95% CI | 37517; 171835 | | 517; 1038 | |
| Week 8 | | | | |
| Geom. Mean | 49361 (3.27) | 6/6 | 744 (1.35) | 0/2 |
| (Geom. Std) | | | | |
| Min; max | 9880; 119000 | | 602; 920 | |
| 95% CI | 19121; 127425 | | 491; 1128 | |
| Week 10 | | | | |
| Geom. Mean | 110984 (4.8) | 6/6 | 633 (1.50) | 0/2 |
| (Geom. Std) | | | | |
| Min; max | 12900; 864000 | | 475; 843 | |
| 95% CI | 31649; 389197 | | 361; 1110 | |
| Week 24 * | | | | |
| Geom. Mean | — | — | — | — |
| (Geom. Std) | | | | |
| Min; max | — | | — | |
| 95% CI | — | | — | |
| Week 26 * | | | | |
| Geom. Mean | — | — | — | — |
| (Geom. Std) | | | | |
| Min; max | — | | — | |
| 95% CI | — | | — | |
| Week 36 | | | | |
| Geom. Mean | 8207 (3.07) | 4/6 | 209 (2.84) | 0/2 |
| (Geom. Std) | | | | |
| Min; max | 1540; 30000 | | 100; 437 | |
| 95% CI | 3347, 20124 | | 49, 887 | |
| Week 48 # | | | | |
| Geom. Mean | 2397 (4.13) # | — | # | — |
| (Geom. Std) | | | | |
| Min; max | 246; 10400 | | | |
| 95% CI | 771, 7448 | | | |
| Week 50 # | | | | |
| Geom. Mean | 13514 (21.84) | — | # | — |
| (Geom. Std) | | | | |
| Min; max | 216; 490000 | | | |
| 95% CI | 1146, 159367 | | | |
| Week 67 # | | | | |
| Geom. Mean | 5030 (5.66) # | NA | # | NA |
| (Geom. Std) | | | | |
| Min; max | — | | | |
| 95% CI | 1256; 20142 | | | |
| Week 74 # | | | | |
| Geom. Mean | 4017 (4.76) # | NA | # | NA |
| (Geom. Std) | | | | |
| Min; max | — | | | |
| 95% CI | 1152; 14008 | | | |

* Due to the Covid-19 pandemic, 7/8 subjects did not receive an immunization of ACI-35.030 (300 μg dose) or placebo at week 24 and hence the data of n = 1 were not reported to avoid potential unblinding. Likewise, no blood sampling was performed at week 26.
# For sub-cohort 1.1, data at weeks 48, 50, 67 and 74 are pooled between active and placebo to avoid potential unblinding, following withdrawal of 2 subjects from the study.
NA = Data not yet available
^{♦}A responder is defined as a subject with an antibody response above the positivity threshold. A post-baseline value is considered positive if ≥ an analytical threshold × baseline. The analytical threshold is defined from samples from human donors (obtained during the validation of each assay). In particular, a responder has anti-pTau IgG titers ≥ 1.81 × baseline.
† Baseline antibody titer value is the mean value of the titers measured at screening and visit 1 (including unscheduled visits) provided that they occur prior to the first injection.

The anti-pTau IgG titers and responder rate (ITT population) following immunization with either ACI-35.030 at the dosage level of 900 μg of tetrapalmitoylated phosphopeptide pTau Peptide T3 (ACI-35.030 900 μg) or placebo in sub-cohort 1.2 are summarized in Table 4.

TABLE 4

| | Sub-cohort 1.2 | | | |
|---|---|---|---|---|
| | ACI-35.030 900 μg | | | |
| Treatment | (n = 6) | | Placebo (n = 2) | |
| groups | Antibody | | Antibody | |
| (number of | titers | Responder | titers | Responder |
| subjects) | (AU/mL) | rate^{♦} | (AU/mL) | rate^{♦} |
| Baseline † | | | | |
| Geom. Mean (Geom. Std) | 1079 (1.93) | — | 778 (1.00) | — |
| Min; max | 542; 3155 | | 776; 780 | |
| 95% CI | 637; 1828 | | 774; 781 | |
| Week 2 | | | | |
| Geom. Mean (Geom. Std) | 321012 (6.81) | 6/6 | 850 (1.10) | 0/2 |
| Min; max | 55600; 9990000 | | 797; 907 | |
| 95% CI | 69198; 1489177 | | 749; 965 | |
| Week 8 | | | | |
| Geom. Mean (Geom. Std) | 141063 (4.52) | 6/6 | 920 (1.00) | 0/2 |
| Min; max | 26600; 1730000 | | 918; 922 | |
| 95% CI | 42200; 471528 | | 916; 924 | |
| Week 10 | | | | |
| Geom. Mean (Geom. Std) | 272400 (2.97) | 6/6 | 673 (1.38) | 0/2 |
| Min; max | 81800; 1510000 | | 535; 847 | |
| 95% CI | 114118; 650220 | | 429; 1056 | |
| Week 24 | | | | |
| Geom. Mean (Geom. Std) | 44635 (2.85) | 6/6 | 934 (1.34) | 0/2 |
| Min; max | 12400; 221000 | | 759; 1150 | |
| 95% CI | 19335; 103040 | | 622; 1404 | |
| Week 26 | | | | |
| Geom. Mean (Geom. Std) | 113771 (1.96) | 6/6 | 1064 (1.08) | 0/2 |
| Min; max | 33500; 234000 | | 1010; 1120 | |
| 95% CI | 66338; 195121 | | 961; 1177 | |
| Week 36 | | | | |
| Geom. Mean (Geom. Std) | 46785 (2.31) | 6/6 | 911 (1.08) | 0/2 |
| Min; max | 12000; 137000 | | 864; 961 | |
| 95% CI | 23966; 91329 | | 821; 1011 | |
| Week 48 | | | | |
| Geom. Mean (Geom. Std) | 29963 (2.21) | 6/6 | 812 (1.13) | 0/2 |
| Min; max | 8270; 80300 | | 743; 887 | |
| 95% CI | 15908; 56435 | | 682; 966 | |
| Week 50 | | | | |
| Geom. Mean (Geom. Std) | 59710 (1.84) | 6/6 | 697 (1.32) | 0/2 |
| Min; max | 31000; 153000 | | 574; 847 | |
| 95% CI | 36666; 97237 | | 476; 1021 | |
| Week 67 | | | | |
| Geom. Mean (Geom. Std) | 24727 (2.00) | 6/6 | 1102 (1.47) | 1/2 |
| Min; max | 11200; 56600 | | 838; 1450 | |
| 95% CI | 14175; 43134 | | 644; 1887 | |
| Week 74 | | | | |
| Geom. Mean (Geom. Std) | 21991 (2.08) | 6/6 | 1187 (1.10) | 0/2 |
| Min; max | 8720; 52900 | | 1110; 1270 | |
| 95% CI | 12264; 39434 | | 1041; 1355 | |

^{♦}A responder is defined as a subject with an antibody response above the positivity threshold. A post-baseline value is considered positive if ≥ an analytical threshold × baseline. The analytical threshold is defined from samples from human donors (obtained during the validation of each assay). In particular, a responder has anti-pTau IgG titers ≥ 1.81 × baseline.
† Baseline antibody titer value is the mean value of the titers measured at screening and visit 1 (including unscheduled visits) provided that they occur prior the date and time of the first injection.

The anti-pTau IgG titers and responder rate (ITT population) following immunization with either ACI-35.030 at the dosage level of 1800 μg of tetrapalmitoylated phosphopeptide pTau Peptide T3 (ACI-35.030 1800 μg) or placebo in sub-cohort 1.3 are summarized in Table 5.

TABLE 5

| | Sub-cohort 1.3 | | | |
|---|---|---|---|---|
| | ACI-35.030 1800 μg | | Placebo | |
| | (n = 6) | | (n = 2) | |
| | Antibody | | Antibody | |
| Treatment groups | titers | Responder | titers | Responder |
| (number of subjects) | (AU/mL) | rate^{♦} | (AU/mL) | rate^{♦} |
| Baseline † | | | | |
| Geom. Mean (Geom. Std) | 676 (1.55) | — | 866 (1.04) | — |
| Min; max | 421; 1130 | | 841; 892 | |
| 95% CI | 476; 961 | | 817; 917 | |
| Week 2 | | | | |
| Geom. Mean (Geom. Std) | 187981 (2.22) | 6/6 | 772 (1.09) | 0/2 |
| Min; max | 71600; 438000 | | 726; 820 | |
| 95% CI | 99157; 356371 | | 685; 869 | |
| Week 8 | | | | |
| Geom. Mean (Geom. Std) | 66036 (2.03) | 6/6 | 719 (1.24) | 0/2 |
| Min; max | 17000; 116000 | | 619; 835 | |
| 95% CI | 37536; 116175 | | 536; 964 | |
| Week 10 | | | | |
| Geom. Mean (Geom. Std) | 135520 (2.21) | 6/6 | 748 (1.07) | 0/2 |
| Min; max | 32700; 337000 | | 715; 782 | |
| 95% CI | 71680; 256219 | | 685; 816 | |
| Week 24 | | | | |
| Geom. Mean (Geom. Std) | 19853 (2.32) | 6/6 | 946 (1.19) | 0/2 |
| Min; max | 4890; 41100 | | 837; 1070 | |
| 95% CI | 38983; 10111 | | 744; 1204 | |
| Week 26 | | | | |
| Geom. Mean (Geom. Std) | 60105 (2.82) | 6/6 | 1070 (1.3) | 0/2 |
| Min; max | 21100; 211000 | | 887; 1290 | |
| 95% CI | 26254; 137604 | | 741; 1544 | |
| Week 31 # | | | | |
| Geom. Mean (Geom. Std) | 30328 # | 100% # | NA | NA |
| Min; max | | | | |
| 95% CI | | | | |
| Week 36 | | | | |
| Geom. Mean (Geom. Std) | 19811 (2.57) | 6/6 | 530 (1.20) | 0/2 |
| Min; max | 5450; 42000 | | 466; 602 | |
| 95% CI | 9322; 42099 | | 412; 681 | |
| Week 42 | | | | |
| Geom. Mean (Geom. Std) | 15349 (2.44) | 6/6 | 712 (1.71) | 0/2 |
| Min; max | 4440; 30200 | | 487; 1040 | |
| 95% CI | 7521; 31327 | | 338; 1497 | |
| Week 48 # | | | | |
| Geom. Mean (Geom. Std) | 10848 # | 100% # | NA | NA |
| Min; max | | | | |
| 95% CI | | | | |
| Week 50 # | | | | |
| Geom. Mean (Geom. Std) | 63736 # | 100% # | NA | NA |
| Min; max | | | | |
| 95% CI | | | | |
| Week 67 | | | | |
| Geom. Mean (Geom. Std) | NA | NA | NA | NA |
| Min; max | | | | |
| 95% CI | | | | |
| Week 74 | | | | |
| Geom. Mean (Geom. Std) | NA | NA | NA | NA |
| Min; max | | | | |
| 95% CI | | | | |

NA = Data not yet available
^{♦}A responder is defined as the number of subjects with an antibody response above the positivity threshold. A post-baseline value is considered positive if ≥ an analytical threshold × baseline. The analytical threshold is defined from samples from human donors (obtained during the validation of each assay). In particular, a responder has anti-pTau IgG titers ≥ 1.81 × baseline
† Baseline antibody titer value is the mean value of the titers measured at screening and visit 1 (including unscheduled visits) provided that they occur prior the date and time of the first injection.
# For sub-cohort 1.3, data at weeks 31, 48 and 50 data from subjects on active treatment are pooled to avoid potential unblinding.

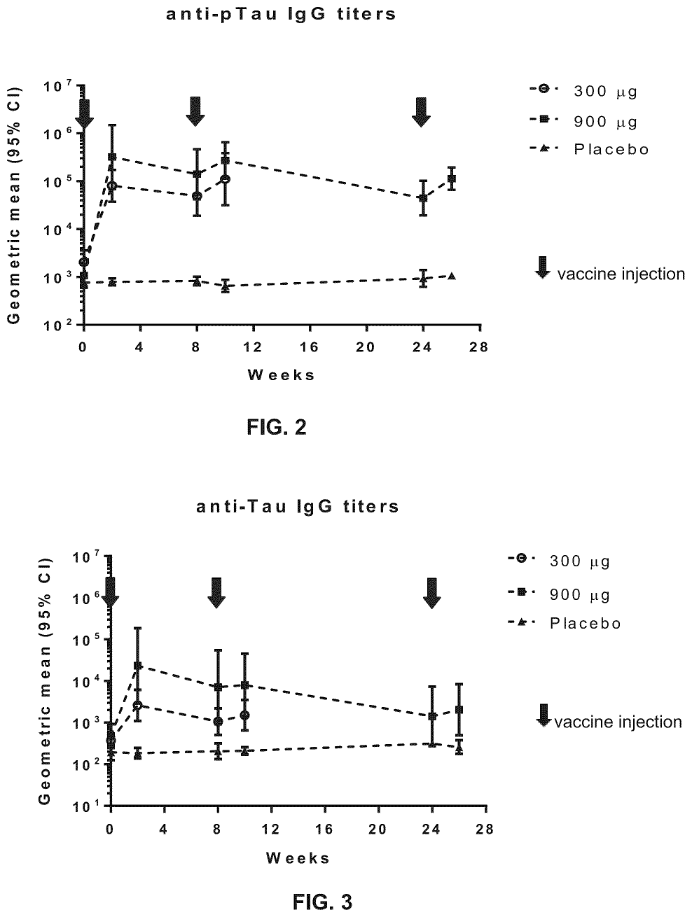

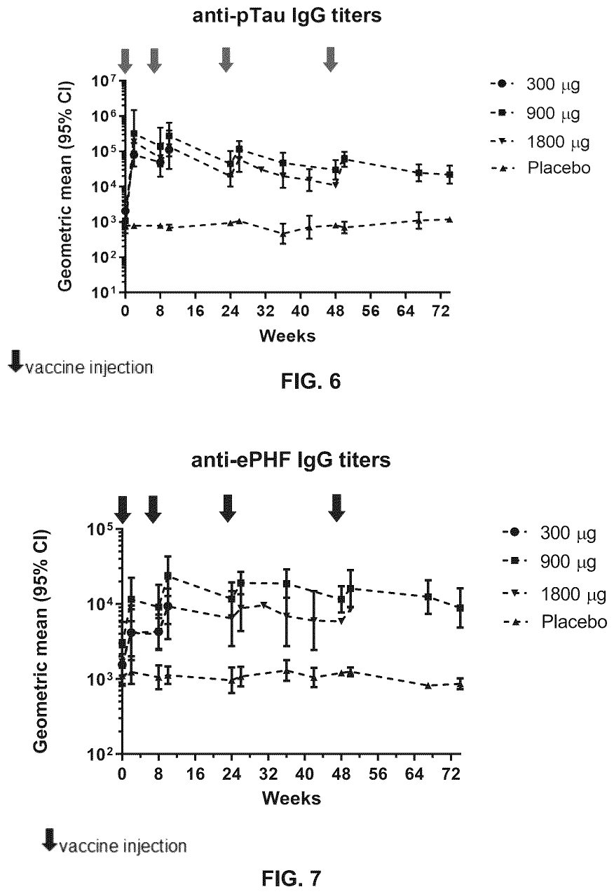

As shown by the results in Tables 3, 4 and 5, and FIGS. 2 and 6, immunization with ACI-35.030 at each of the 300 μg, 900 μg and 1800 μg dose levels induced an anti-pTau IgG response directed against the T3.5 peptide having the amino acid sequence of SEQ ID NO: 28. All subjects treated with ACI-35.030 at the 300 μg dose level were responders from week 2 to week 10, and 66.7% of them were responders at week 36 whereas prior injection at week 24 was not performed in 7/8 subjects in the sub-cohort due to the Covid-19 pandemic. In order to avoid potential study unblinding, the percentage of responders beyond week 36 in the sub-cohort 1 are not reported for the moment. All subjects treated with ACI-35.030 at the 900 μg dose level were responders at all timepoints between week 2 and week 74, while 1 subject receiving placebo generated a limited anti-pTau IgG response at week 67 (after the treatment period) (1.9×baseline) just slightly higher than the threshold (1.81×baseline) set for defining a responder All subjects treated with ACI-35.030 at the 1800 μg dose level were responders at all timepoints between week 2 and week 50, while no subjects receiving placebo generated an anti-pTau IgG response up to week 42.

High responder rates were observed as early as 2 weeks post vaccination for subjects treated with either 300 μg or 900 μg of ACI-35.030. Overall, high responder rates were observed after the first vaccination and following all vaccinations. At the 900 μg dose of ACI-35.030, there was a 100% responder rate for phosphorylated Tau when analyzed at any study timepoint from week 2 to week 74. The responder rate for pathological ePHF at the 900 μg dose of ACI-35.030 ranged from 66.7% to 100% at any timepoint during the treatment period between week 2 and week 48 and from 50% to 100% at any timepoint during the post-treatment period from week 50 to week 74. Furthermore, rapid class-switching was observed from IgM to IgG in patients treated with either the 300 μg or 900 μg of ACI-35.030. A summary of the overall response rates for sub-cohorts 1.1., 1.2 and 1.3 treated with 300 μg, 900 μg or 1800 μg of ACI-35.030, respectively is shown in Table 6.

TABLE 6

| | Week | Week | Week | Week | Week |
|---|---|---|---|---|---|
| | 2 | 10 | 26 | 50 | 74 |
| | Sub-cohort 1.1 (300 μg (i.m.)) | | | | |
| Anti- | 83.3% | 33.3% | NA | NA | NA |
| Tau | | | | | |
| IgG | | | | | |
| Anti- | 100% | 100% | NA | NA | NA |
| pTau | | | | | |
| IgG | | | | | |
| Anti- | 66.7% | 83.3% | NA | NA | NA |
| ePHF | | | | | |
| IgG | | | | | |
| | Sub-cohort 1.2 (900 μg (i.m.)) | | | | |
| Anti- | 83.3% | 100% | 50% | 16.7% | 16.7% |
| Tau | | | | | |
| IgG | | | | | |
| Anti- | 100% | 100% | 100% | 100% | 100% |
| pTau | | | | | |
| IgG | | | | | |
| Anti- | 100% | 100% | 100% | 100% | 50% |
| ePHF | | | | | |
| IgG | | | | | |
| | Sub-cohort 1.3 (1800 μg (i.m.)) | | | | |
| Anti- | 83.3% | 66.7% | 50% | 80% | NA |
| Tau | | | | | |
| IgG | | | | | |
| Anti- | 100% | 100% | 100% | 100% | NA |
| pTau | | | | | |
| IgG | | | | | |
| Anti- | 66.7% | 83.3% | 66.7% | 80% | NA |
| ePHF | | | | | |
| IgG | | | | | |

Responders were defined as subjects with an antibody response higher than a positivity threshold, i.e., a pretreatment value (baseline antibody titer), multiplied by a threshold factor (>~2×). Any post-baseline result greater than or equal to this value defines a positive antibody response. Baseline antibody titer value is the mean value of the titers measured at screening and visit 1 (including unscheduled visits) provided that they occur prior to the first injection. In particular, a responder has anti-Tau IgG titers ≥ 3.38 × baseline, anti-pTau IgG titers ≥ 1.81 × baseline, and/or anti-ePHF IgG titers ≥ 2.21 × baseline.
NA = data not yet available

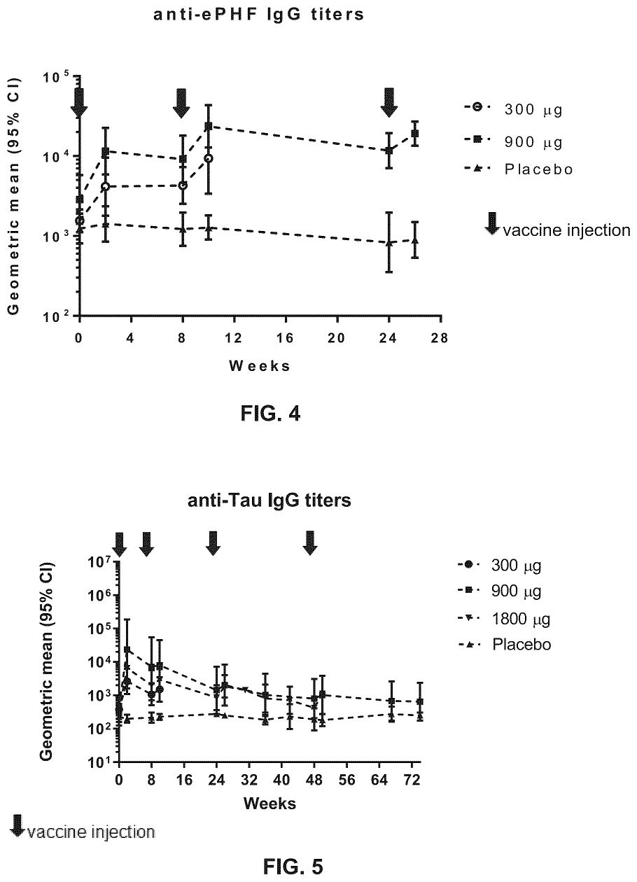

In general, each additional immunization at weeks 8, 24 or 48 at both the 300 and 900 μg dose levels led to a boosting of the anti-pTau IgG response as shown by increased anti-pTau-specific IgG titers measured 2 weeks after the administration of the additional immunization, at weeks 10, 26 and 50, respectively (FIGS. 2 and 6). Additional immunization at weeks 8, 24, and 48 with the 1800 μg dose level also led to a boosting of the anti-pTau IgG response as shown by increased anti-pTau-specific IgG titers measured 2 weeks after the administration of the additional immunization at weeks 10, 26 and 50 (FIGS. 4 and 7).

### Anti-pTau IgM Response of ACI-35.030 in Humans

Specific IgM antibody responses directed against the phosphorylated Tau peptide induced by ACI-35.030 vaccine in the three sub-cohorts of Table 2 were measured by MSD. The anti-pTau IgM titers and responder rate (ITT population) following immunization with either ACI-35.030 300 μg or placebo in sub-cohort 1.1 are shown in Table 7.

TABLE 7

| | Sub-cohort 1.1 | | | |
|---|---|---|---|---|
| | ACI-35.030 300 μg | | Placebo | |
| | (n = 6) | | (n = 2) | |
| | Antibody | | Antibody | |
| Treatment groups | titers | Responder | titers | Responder |
| (number of subjects) | (AU/mL) | rate^{♦} | (AU/mL) | rate^{♦} |
| Baseline † | | | | |
| Geom. Mean (Geom. Std) | 355 (2.66) | — | 442 (1.16) | — |
| Min; max | 111; 1720 | | 397; 492 | |
| 95% CI | 162, 777 | | 358, 545 | |
| Week 2 | | | | |
| Geom. Mean (Geom. Std) | 25952 (2.66) | 6/6 | 459 (1.31) | 0/2 |
| Min; max | 8690; 92200 | | 380; 555 | |
| 95% CI | 11859, 56794 | | 317, 666 | |
| Week 8 | | | | |
| Geom. Mean (Geom. Std) | 3667 (2.44) | 6/6 | 400 (1.23) | 0/2 |
| Min; max | 1140; 14500 | | 346; 462 | |
| 95% CI | 1795, 7489 | | 301, 531 | |
| Week 10 | | | | |
| Geom. Mean (Geom. Std) | 4437 (1.84) | 6/6 | 421 (1.25) | 0/2 |
| Min; max | 2070; 11300 | | 361, 492 | |
| 95% CI | 2723, 7229 | | 311, 571 | |
| Week 24 * | | | | |
| Geom. Mean (Geom. Std) | — | — | — | — |
| Min; max | — | | — | |
| 95% CI | — | | — | |
| Week 26 * | | | | |
| Geom. Mean (Geom. Std) | — | — | — | — |
| Min; max | — | | — | |
| 95% CI | — | | — | |
| Week 36 | | | | |
| Geom. Mean (Geom. Std) | 806 (2.44) | 3/6 | 365 (1.27) | 0/2 |
| Min; max | 187; 2560 | | 309; 431 | |
| 95% CI | 394, 1648 | | 263, 506 | |
| Week 48 # | | | | |
| Geom. Mean (Geom. Std) | 625 (2.04) # | — | # | — |
| Min; max | 308; 2150 | | | |
| 95% CI | 353, 1106 | | | |
| Week 50 # | | | | |
| Geom. Mean (Geom. Std) | 2138 (4.16) # | — | # | — |
| Min; max | 293; 10600 | | | |
| 95% CI | 684, 6681 | | | |
| Week 67 # | | | | |
| Geom. Mean (Geom. Std) | 1049 (3.03) # | NA | # | NA |
| Min; max | — | | | |
| 95% CI | 432; 2547 | | | |
| Week 74 # | | | | |
| Geom. Mean (Geom. Std) | 1077 (2.91) # | NA | # | NA |
| Min; max | — | | | |
| 95% CI | 458; 2534 | | | |

* Due to the Covid-19 pandemic, 7/8 subjects did not receive an immunization of ACI-35.030 (300 μg dose) or placebo at week 24 and hence the data of n = 1 were not reported to avoid potential unblinding. Likewise, no blood sampling was performed at week 26.
# For sub-cohort 1.1, data at weeks 48, 50, 67 and 74 are pooled between active and placebo to avoid potential unblinding, following withdrawal of 2 subjects from the study.
NA = Data not yet available
^{♦}A responder is defined as a subject with an antibody response above the positivity threshold. A post-baseline value is considered positive if ≥ an analytical threshold × baseline. The analytical threshold is defined from samples from human donors (obtained during the validation of each assay). In particular, a responder has anti-pTau IgM titers ≥ 2.11 × baseline.
† Baseline antibody titer value is the mean value of the titers measured at screening and visit 1 (including unscheduled visits) provided that they occur prior to the first injection.

The anti-pTau IgM titers and responder rate (ITT population) following immunization with either ACI-35.030 900 μg or placebo in sub-cohort 1.2 are shown in Table 8.

TABLE 8

| | Sub-cohort 1.2 | | | |
|---|---|---|---|---|
| | ACI-35.030 900 μg | | Placebo | |
| | (n = 6) | | (n = 2) | |
| | Antibody | | Antibody | |
| Treatment groups | titers | Responder | titers | Responder |
| (number of subjects) | (AU/mL) | rate^{♦} | (AU/mL) | rate^{♦} |
| Baseline † | | | | |
| Geom. Mean (Geom. Std) | 200 (2.23) | — | 82 (2.01) | — |
| Min; max | 50; 448 | | 50; 134 | |
| 95% CI | 105, 379 | | 31, 215 | |
| Week 2 | | | | |
| Geom. Mean (Geom. Std) | 29736 (2.14) | 6/6 | 80 (1.94) | 0/2 |
| Min; max | 14000; 117000 | | 50; 128 | |
| 95% CI | 16155, 54736 | | 32, 201 | |
| Week 8 | | | | |
| Geom. Mean (Geom. Std) | 6157 (1.94) | 6/6 | 84 (2.08) | 0/2 |
| Min; max | 2730; 18300 | | 50; 141 | |
| 95% CI | 3629, 10446 | | 30, 232 | |
| Week 10 | | | | |
| Geom. Mean (Geom. Std) | 8546 (1.78) | 6/6 | 84 (2.09) | 0/2 |
| Min; max | 3430; 17100 | | 50; 142 | |
| 95% CI | 5394, 13540 | | 30, 234 | |
| Week 24 | | | | |
| Geom. Mean (Geom. Std) | 2375 (1.68) | 6/6 | 96 (2.53) | 0/2 |
| Min; max | 1690; 6390 | | 50; 186 | |
| 95% CI | 1565, 3605 | | 27, 349 | |
| Week 26 | | | | |
| Geom. Mean (Geom. Std) | 5317 (2.09) | 6/6 | 90 (2.31) | 0/2 |
| Min; max | 2020; 10200 | | 50; 163 | |
| 95% CI | 2952, 9576 | | 28, 287 | |
| Week 36 | | | | |
| Geom. Mean (Geom. Std) | 3111 (2.48) | 6/6 | 78 (1.88) | 0/2 |
| Min; max | 816; 7770 | | 50; 122 | |
| 95% CI | 1502; 6445 | | 33; 187 | |
| Week 48 | | | | |
| Geom. Mean (Geom. Std) | 1728 (2.32) | 6/6 | 77 (1.85) | 0/2 |
| Min; max | 510; 4310 | | 50; 119 | |
| 95% CI | 882; 3386 | | 33; 180 | |
| Week 50 | | | | |
| Geom. Mean (Geom. Std) | 4228 (2.95) | 6/6 | 71 (1.64) | 0/2 |
| Min; max | 1420; 23900 | | 50; 101 | |
| 95% CI | 1779; 10048 | | 36, 142 | |
| Week 67 | | | | |
| Geom. Mean (Geom. Std) | 1746 (3.07) | 6/6 | 164 (5.34) | 1/2 |
| Min; max | 414; 6220 | | 50; 535 | |
| 95% CI | 712; 4282 | | 16; 1669 | |
| Week 74 | | | | |
| Geom. Mean (Geom. Std) | 1471 (2.65) | 6/6 | 106 (2.88) | 0/2 |
| Min; max | 461; 4540 | | 50.0; 223.0 | |
| 95% CI | 674; 3209 | | 24; 457 | |

^{♦}A responder is defined as a subject with an antibody response above the positivity threshold. A post-baseline value is considered positive if ≥ an analytical threshold × baseline. The analytical threshold is defined from samples from human donors (obtained during the validation of each assay). In particular, a responder has anti-pTau IgM titers ≥ 2.11 × baseline.
† Baseline antibody titer value is the mean value of the titers measured at screening and visit 1 (including unscheduled visits) provided that they occur prior to the first injection.

The anti-pTau IgM titers and responder rate (ITT population) following immunization with either ACI-35.030 1800 μg or Placebo in sub-cohort 1.3 are shown in Table 9.

TABLE 9

| | Sub-cohort 1.3 | | | |
|---|---|---|---|---|
| | ACI-35.030 1800 μg | | Placebo | |
| | (n = 6) | | (n = 2) | |
| | Antibody | | Antibody | |
| Treatment groups | titers | Responder | titers | Responder |
| (number of subjects) | (AU/mL) | rate^{♦} | (AU/mL) | rate^{♦} |
| Baseline † | | | | |
| Geom. Mean (Geom. Std) | 369 (1.64) | — | 504 (1.96) | — |
| Min; max | 224; 842 | | 313; 812 | |
| 95% CI | 248; 548 | | 198; 1283 | |
| Week 2 | | | | |
| Geom. Mean (Geom. Std) | 20464 (4.21) | 6/6 | 491 (1.96) | 0/2 |
| Min; max | 5240; 180000 | | 306; 788 | |
| 95% CI | 6475; 64676 | | 194; 1241 | |
| Week 8 | | | | |
| Geom. Mean (Geom. Std) | 4937 (4.01) | 6/6 | 422 (1.90) | 0/2 |
| Min; max | 1410; 48600 | | 268; 665 | |
| 95% CI | 1625; 14995 | | 173; 1029 | |
| Week 10 | | | | |
| Geom. Mean (Geom. Std) | 7673 (2.73) | 6/6 | 473 (1.94) | 0/2 |
| Min; max | 2080; 31200 | | 296; 757 | |
| 95% CI | 3434; 17145 | | 189; 1188 | |
| Week 24 | | | | |
| Geom. Mean (Geom. Std) | 2758 (2.38) | 6/6 | 710 (3.05) | 0/2 |
| Min; max | 1290; 13700 | | 323; 1560 | |
| 95% CI | 1380; 5513 | | 152; 3322 | |
| Week 26 | | | | |
| Geom. Mean (Geom. Std) | 7950 (2.47) | 6/6 | 800 (2.73) | 0/2 |
| Min; max | 2920; 30900 | | 393; 1630 | |
| 95% CI | 3858; 16384 | | 199; 3226 | |
| Week 31 # | | | | |
| Geom. Mean (Geom. Std) | 4061 # | 100% # | NA | NA |
| Min; max | | | | |
| 95% CI | | | | |
| Week 36 | | | | |
| Geom. Mean (Geom. Std) | 2906 (2.25) | 6/6 | 327 (3.32) | 0/2 |
| Min; max | 1360; 13100 | | 140; 765 | |
| 95% CI | 1517; 5569 | | 62; 1729 | |
| Week 42 | | | | |
| Geom. Mean (Geom. Std) | 2298 (2.29) | 6/6 | 397 (2.43) | 0/2 |
| Min; max | 1020; 10800 | | 212; 745 | |
| 95% CI | 1183; 4462 | | 116; 1362 | |
| Week 48 # | | | | |
| Geom. Mean (Geom. Std) | 2363 # | 100% | NA | NA |
| Min; max | | | | |
| 95% CI | | | | |
| Week 50 # | | | | |
| Geom. Mean (Geom. Std) | 8509 # | 100% | NA | NA |
| Min; max | | | | |
| 95% CI | | | | |
| Week 67 | | | | |
| Geom. Mean (Geom. Std) | NA | NA | NA | NA |
| Min; max | | | | |
| 95% CI | | | | |
| Week 74 | | | | |
| Geom. Mean (Geom. Std) | NA | NA | NA | NA |
| Min; max | | | | |
| 95% CI | | | | |

NA = Data not yet available
^{♦}A responder is defined as the number of subjects with an antibody response above the positivity threshold. A post-baseline value is considered positive if ≥ an analytical threshold × baseline. The analytical threshold is defined from samples from human donors (obtained during the validation of each assay). In particular, a responder has anti-pTau IgM titers ≥ 2.11 × baseline.
† Baseline antibody titer value is the mean value of the titers measured at screening and visit 1 (including unscheduled visits) provided that they occur prior to the first injection.
# For sub-cohort 1.3, data at weeks 31, 48 and 50 data from subjects on active treatment are pooled to avoid potential unblinding

As shown by the results of Tables 7 to 9, immunization of early AD subjects with ACI-35.030 at each of the 300 μg, 900 μg and 1800 μg doses induced an anti-pTau IgM response by week 10 directed against the T3.5 peptide having the amino acid sequence of SEQ ID NO:28. All subjects treated with ACI-35.030 were responders, while no subjects treated with placebo generated an anti-pTau IgM response, except for 1 subject at week 67 (after the treatment period), who showed a limited anti-pTau IgM response just slightly higher than the threshold set for defining a responder. Together, the decline of the anti-pTau IgM response by week 8, and the anti-pTau IgG antibody response measured from week 2 onwards suggests that ACI-35.030 induced an IgM to IgG class switch.

### Specificity Against pTau Over Tau (Non-pTau)

See FIGS. 3 and 5 for the IgG titers against nonphosphorylated Tau (anti-Tau IgG titers) induced by the various dosages of ACI-35.030 or placebo over time. The specificity of the IgG antibody response induced by immunization with ACI-35.030 in the three sub-cohorts of Table 2 for binding to pTau over Tau (where the response to Tau represents the response to non-pTau) was measured as the ratio of the anti-pTau IgG/anti-Tau IgG response over time. The anti-Tau IgG titers and responder rate (ITT population) following immunization with either ACI-35.030 300 μg or placebo in sub-cohort 1.1 are shown in Table 10.

TABLE 10

| | Sub-cohort 1.1 | | | |
|---|---|---|---|---|
| | ACI-35.030 300 μg | | Placebo | |
| | (n = 6) | | (n = 2) | |
| | Antibody | | Antibody | |
| Treatment groups | titers | Responder | titers | Responder |
| (number of subjects) | (AU/mL) | rate^{♦} | (AU/mL) | rate^{♦} |
| Baseline † | | | | |
| Geom. Mean (Geom. Std) | 375 (1.39) | — | 154 (1.50) | — |
| Min; max | 295; 705 | | 116; 206 | |
| 95% CI | 287, 489 | | 88, 271 | |
| Week 2 | | | | |
| Geom. Mean (Geom. Std) | 2609 (2.95) | 5/6 | 147 (1.20) | 0/2 |
| Min; max | 723; 15400 | | 129; 167 | |
| 95% CI | 1099, 6197 | | 114, 189 | |
| Week 8 | | | | |
| Geom. Mean (Geom. Std) | 1065 (2.5) | 2/6 | 152 (1.54) | 0/2 |
| Min; max | 385; 5200 | | 112; 206 | |
| 95% CI | 512, 2214 | | 84, 276 | |
| Week 10 | | | | |
| Geom. Mean (Geom. Std) | 1509 (2.87) | 2/6 | 192 (1.16) | 0/2 |
| Min; max | 524; 9340 | | 173; 213 | |
| 95% CI | 649, 3505 | | 157, 235 | |
| Week 24 * | | | | |
| Geom. Mean (Geom. Std) | — | — | — | — |
| Min; max | — | | — | |
| 95% CI | — | | — | |
| Week 26 * | | | | |
| Geom. Mean (Geom. Std) | — | — | — | — |
| Min; max | — | | — | |
| 95% CI | — | | — | |
| Week 36 | | | | |
| Geom. Mean (Geom. Std) | 523 (1.61) | 1/6 | 158 (1.76) | 0/2 |
| Min; max | 288; 1130 | | 106; 235 | |
| 95% CI | 358, 764 | | 72, 344 | |
| Week 48 # | | | | |
| Geom. Mean (Geom. Std) | 303 (2) # | — | # | — |
| Min; max | 123; 572 | | | |
| 95% CI | 186, 493 | | | |
| Week 50 # | | | | |
| Geom. Mean (Geom. Std) | 532 (3) # | — | # | — |
| Min; max | 102; 2310 | | | |
| 95% CI | 217, 1305 | | | |
| Week 67 # | | | | |
| Geom. Mean (Geom. Std) | 297 (1.99) # | NA | # | NA |
| Min; max | — | | | |
| 95% CI | 171; 514 | | | |
| Week 74 # | | | | |
| Geom. Mean (Geom. Std) | 265 (2.46) # | NA | # | NA |
| Min; max | — | | | |
| 95% CI | 129; 544 | | | |

* Due to the Covid-19 pandemic, 7/8 subjects did not receive an immunization of ACI-35.030 (300 μg dose) or placebo at week 24 and hence the data of n = 1 were not reported to avoid potential unblinding. Likewise, no blood sampling was performed at week 26.
# For sub-cohort 1.1, data at weeks 48, 50, 67 and 74 are pooled between active and placebo to avoid potential unblinding, following withdrawal of 2 subjects from the study.
NA = Data not yet available
^{♦}A responder is defined as a subject with an antibody response above the positivity threshold. A post-baseline value is considered positive if ≥ an analytical threshold × baseline. The analytical threshold is defined from samples from human donors (obtained during the validation of each assay). In particular, a responder has anti-Tau IgG titers ≥ 3.38 × baseline.
† Baseline antibody titer value is the mean value of the titers measured at screening and visit 1 (including unscheduled visits) provided that they occur prior to the first injection.

The anti-Tau IgG titers and responder rate (ITT population) following immunization with either ACI-35.030 900 μg or placebo in sub-cohort 1.2 are shown in Table 11.

TABLE 11

| | Sub-cohort 1.2 | | | |
|---|---|---|---|---|
| | ACI-35.030 900 μg | | Placebo | |
| | (n = 6) | | (n = 2) | |
| | Antibody | | Antibody | |
| Treatment groups | titers | Responder | titers | Responder |
| (number of subjects) | (AU/mL) | rate^{♦} | (AU/mL) | rate^{♦} |
| Baseline † | | | | |
| Geom. Mean (Geom. Std) | 477 (2.26) | — | 242 (1.54) | — |
| Min; max | 209; 1960 | | 179; 328 | |
| 95% CI | 249, 914 | | 133, 438 | |
| Week 2 | | | | |
| Geom. Mean (Geom. Std) | 23242 (13.53) | 5/6 | 235 (1.19) | 0/2 |
| Min; max | 1950; 3550000 | | 208; 266 | |
| 95% CI | 2891, 186826 | | 185, 299 | |
| Week 8 | | | | |
| Geom. Mean (Geom. Std) | 7108 (12.79) | 4/6 | 277 (1.30) | 0/2 |
| Min; max | 1070; 1150000 | | 230; 333 | |
| 95% CI | 925, 54634 | | 193, 398 | |
| Week 10 | | | | |
| Geom. Mean (Geom. Std) | 7993 (8.65) | 6/6 | 230 (1.35) | 0/2 |
| Min; max | 1700; 607000 | | 186; 284 | |
| 95% CI | 1423, 44914 | | 152, 348 | |
| Week 24 | | | | |
| Geom. Mean (Geom. Std) | 1423 (7.67) | 1/6 | 313 (1.07) | 0/2 |
| Min; max | 520; 88400 | | 299; 327 | |
| 95% CI | 279, 7266 | | 286, 341 | |
| Week 26 | | | | |
| Geom. Mean (Geom. Std) | 2025 (5.89) | 3/6 | 257 (1.31) | 0/2 |
| Min; max | 676; 70400 | | 212; 312 | |
| 95% CI | 490, 8370 | | 176, 376 | |
| Week 36 | | | | |
| Geom. Mean (Geom. Std) | 1032 (6.21) | 1/6 | 203 (1.61) | 0/2 |
| Min; max | 285; 39900 | | 145; 283 | |
| 95% CI | 239, 4451 | | 105; 390 | |
| Week 48 | | | | |
| Geom. Mean (Geom. Std) | 798 (5.48) | 1/6 | 190.45 (1.72) | 0/2 |
| Min; max | 238; 24200 | | 130; 279 | |
| 95% CI | 205; 3113 | | 90; 403 | |
| Week 50 | | | | |
| Geom. Mean (Geom. Std) | 1019 (5.21) | 1/6 | 182 (1.36) | 0/2 |
| Min; max | 367; 28600 | | 146; 225 | |
| 95% CI | 272; 3819 | | 119; 277 | |
| Week 67 | | | | |
| Geom. Mean (Geom. Std) | 682 (5.45) | 1/6 | 275 (1.46) | 0/2 |
| Min; max | 242; 20500 | | 210; 359 | |
| 95% CI | 176, 2646 | | 162; 464 | |
| Week 74 | | | | |
| Geom. Mean (Geom. Std) | 648 (5.06) | 1/6 | 251 (1.16) | 0/2 |
| Min; max | 236; 16500 | | 226; 278 | |
| 95% CI | 177; 2371 | | 205; 307 | |

^{♦}A responder is defined as a subject with an antibody response above the positivity threshold. A post-baseline value is considered positive if ≥ an analytical threshold × baseline. The analytical threshold is defined from samples from human donors (obtained during the validation of each assay). In particular, a responder has anti-Tau IgG titers ≥ 3.38 × baseline.
† Baseline antibody titer value is the mean value of the titers measured at screening and visit 1 (including unscheduled visits) provided that they occur prior the date and time of the first injection.

The anti-Tau IgG titers and responder rate (ITT population) following immunization with either ACI-35.030 1800 μg or placebo in sub-cohort 1.3 are shown in Table 12.

TABLE 12

| | Sub-cohort 1.3 | | | |
|---|---|---|---|---|
| | ACI-35.030 1800 microg | | Placebo | |
| | (n = 6) | | (n = 2) | |
| | Antibody | | Antibody | |
| Treatment groups | titers | Responder | titers | Responder |
| (number of subjects) | (AU/mL) | rate^{♦} | (AU/mL) | rate^{♦} |
| Baseline † | | | | |
| Geom. Mean (Geom. Std) | 355 (3.68) | — | 306 (1.45) | — |
| Min; max | 81; 3780 | | 235; 399 | |
| 95% CI | 125; 1009 | | 182; 514 | |
| Week 2 | | | | |
| Geom. Mean (Geom. Std) | 6550 (5.75) | 5/6 | 228 (1.63) | 0/2 |
| Min; max | 660; 50800 | | 162; 322 | |
| 95% CI | 1615; 26565 | | 116; 448 | |
| Week 8 | | | | |
| Geom. Mean (Geom. Std) | 2001 (3.69) | 3/6 | 238 (1.71) | 0/2 |
| Min; max | 432; 11500 | | 163; 347 | |
| 95% CI | 704; 5685 | | 113; 499 | |
| Week 10 | | | | |
| Geom. Mean (Geom. Std) | 2922 (2.53) | 4/6 | 265 (1.45) | 0/2 |
| Min; max | 1120; 9340 | | 204; 344 | |
| 95% CI | 1392; 6134 | | 159; 442 | |
| Week 24 | | | | |
| Geom. Mean (Geom. Std) | 852 (2.89) | 1/6 | 246 (1.03) | 0/2 |
| Min; max | 251; 4750 | | 241; 252 | |
| 95% CI | 364; 1990 | | 236; 257 | |
| Week 26 | | | | |
| Geom. Mean (Geom. Std) | 1859 (2.59) | 3/6 | 246 (1.04) | 0/2 |
| Min; max | 491; 5420 | | 239; 253 | |
| 95% CI | 868; 3982 | | 233; 260 | |
| Week 31 # | | | | |
| Geom. Mean (Geom. Std) | 1437 # | 40% # | NA | NA |
| Min; max | | | | |
| 95% CI | | | | |
| Week 36 | | | | |
| Geom. Mean (Geom. Std) | 806 (3.35) | 1/6 | 202 (1.49) | 0/2 |
| Min; max | 237; 6070 | | 153; 268 | |
| 95% CI | 306; 2121 | | 117; 351 | |
| Week 42 | | | | |
| Geom. Mean (Geom. Std) | 709 (3.26) | 1/6 | 230 (1.86) | 0/2 |
| Min; max | 175; 5360 | | 148; 356 | |
| 95% CI | 275; 1824 | | 97; 543 | |
| Week 48 # | | | | |
| Geom. Mean (Geom. Std) | 417 # | 20% # | NA | NA |
| Min; max | | | | |
| 95% CI | | | | |
| Week 50 # | | | | |
| Geom. Mean (Geom. Std) | 1133 # | 80% # | NA | NA |
| Min; max | | | | |
| 95% CI | | | | |
| Week 67 | | | | |
| Geom. Mean (Geom. Std) | NA | NA | NA | NA |
| Min; max | | | | |
| 95% CI | | | | |
| Week 74 | | | | |
| Geom. Mean (Geom. Std) | NA | NA | NA | NA |
| Min; max | | | | |
| 95% CI | | | | |

NA = Data not yet available
^{♦}A responder is defined as the number of subjects with an antibody response above the positivity threshold. A post-baseline value is considered positive if ≥ an analytical threshold × baseline. The analytical threshold is defined from samples from human donors (obtained during the validation of each assay). In particular, a responder has anti-Tau IgG titers ≥ 3.38 × baseline.
† Baseline antibody titer value is the mean value of the titers measured at screening and visit 1 (including unscheduled visits) provided that they occur prior the date and time of the first injection.
# For sub-cohort 1.3, data at weeks 31, 48 and 50 data from subjects on active treatment are pooled to avoid potential unblinding
NA = Data not yet available
* Due to the Covid-19 pandemic, 7/8 subjects did not receive an immunization of ACI-35.030 (300 μg dose) or placebo at week 24 and hence the data of n = 1 were not reported to avoid study unblinding. Likewise, no blood sampling was performed at week 26.

The ratio of the anti-pTau IgG/anti-Tau IgG titers (ITT population) following immunization with ACI-35.030 300 μg in sub-cohort 1 is shown in Table 13.

TABLE 13

| | | | Ratio geometric |
|---|---|---|---|
| | Geometric mean | Geometric mean | mean anti-pTau |
| | anti-pTau IgG | anti-Tau IgG | IgG/anti-Tau |
| | titers (AU/mL) | titers (AU/mL) | IgG titers (AU/mL) |
| | 300 μg dose | 300 μg dose | 300 μg dose |
| Week | ACI-35.030 | ACI-35.030 | ACI-35.030 |
| Baseline | 2048 | 375 | 5.5 |
| 2 | 80292 | 2610 | 30.8 |
| 8 | 49361 | 1065 | 46.4 |
| 10 | 110984 | 1509 | 73.6 |
| 24 * | * | * | — |
| 26 * | * | * | — |
| 36 | 8207 | 523 | 15.7 |
| 48 # | 2397 # | 303 # | 7.9 |
| 50 # | 13514 # | 532 # | 25.4 |
| 67 # | 5030 # | 297 # | 16.9 |
| 74 # | 4018 # | 265 # | 15.2 |

NA = Data not yet available
* Due to the Covid-19 pandemic, 7/8 subjects did not receive an immunization of ACI-35.030 (300 μg dose) or placebo at week 24 and hence the data of n = 1 were not reported to avoid study unblinding. Likewise, no blood sampling was performed at week 26.
# For sub-cohort 1.1, data at weeks 48, 50, 67, and 74 are pooled between active and placebo to avoid study unblinding, following withdrawal of 2 subjects from the study

Table 14 shows the ratio of the anti-pTau IgG/anti-Tau IgG titers (ITT population) following immunization with ACI-35.030 900 μg in sub-cohort 1.2.

TABLE 14

| | | | Ratio geometric |
|---|---|---|---|
| | Geometric mean | Geometric mean | mean anti-pTau |
| | anti-pTau IgG | anti-Tau IgG | IgG/anti-Tau |
| | titers (AU/mL) | titers (AU/mL) | IgG titers (AU/mL) |
| | 900 μg dose | 900 μg dose | 900 μg dose |
| Week | ACI-35.030 | ACI-35.030 | ACI-35.030 |
| Baseline | 1079 | 477 | 2.3 |
| 2 | 321012 | 23242 | 13.8 |
| 8 | 141063 | 7108 | 19.9 |
| 10 | 272400 | 7993 | 34.1 |
| 24 | 44635 | 1423 | 31.4 |
| 26 | 113771 | 2025 | 56.2 |
| 36 | 46785 | 1032 | 45.3 |
| 48 | 29963 | 798 | 37.5 |
| 50 | 59710 | 1019 | 58.6 |
| 67 | 24727 | 682 | 36.3 |
| 74 | 21991 | 648 | 33.9 |

Table 15 shows the ratio of the anti-pTau IgG/anti-Tau IgG titers (ITT population) following immunization with ACI-35.030 1800 μg in sub-cohort 1.3.

TABLE 15

| | | | Ratio geometric |
|---|---|---|---|
| | Geometric mean | Geometric mean | mean anti-pTau |
| | anti-pTau IgG | anti-Tau IgG | IgG/anti-Tau |
| | titers (AU/mL) | titers (AU/mL) | IgG titers (AU/mL) |
| | 1800 μg | 1800 μg | 1800 μg |
| Week | dose ACI-35.030 | dose ACI-35.030 | dose ACI-35.030 |
| Baseline | 676 | 355 | 2.0 |
| 2 | 187981 | 6550 | 28.7 |
| 8 | 66036 | 2001 | 33.0 |
| 10 | 135520 | 2922 | 46.4 |
| 24 | 19853 | 852 | 23.3 |
| 26 | 60105 | 1859 | 32.3 |
| 31 | 30328 | 1437 | 21.1 |
| 36 | 19811 | 806 | 24.6 |
| 42 | 15349 | 709 | 21.7 |
| 48 | 10848 | 417 | 26.0 |
| 50 | 63736 | 1133 | 56.3 |
| 67 | NA | NA | NA |
| 74 | NA | NA | NA |

NA = Data not yet available

As shown by the results of Tables 13 to 15, immunization with ACI-35.030 at each of the 300 μg, 900 μg and 1800 μg doses induced an IgG antibody response, which preferentially recognizes pTau peptide over non-pTau peptide and this preference lasted over time.

### Recognition of Pathological pTau (enriched Paired Helical Filaments—ePHF) Derived from Human AD Brain

The ability of the IgG polyclonal antibodies induced by immunization with ACI-35.030 in the three sub-cohorts of Table 2 to bind to ePHF derived from human AD brain was measured over time by MSD. Table 16 shows the anti-ePHF IgG titers and responder rate (ITT population) following immunization with either ACI-35.030 300 μg or placebo in sub-cohort 1.1.

TABLE 16

| | Sub-cohort 1.1 | | | |
|---|---|---|---|---|
| | ACI-35.030 300 μg | | Placebo | |
| | (n = 6) | | (n = 2) | |
| | Antibody | | Antibody | |
| Treatment groups | titers | Responder | titers | Responder |
| (number of subjects) | (AU/mL) | rate^{♦} | (AU/mL) | rate^{♦} |
| Baseline † | | | | |
| Geom. Mean (Geom. Std) | 1553 (1.51) | — | 1657 (1.57) | — |
| Min; max | 897; 3080 | | 1205; 2275 | |
| 95% CI | 1117, 2160 | | 888, 3086 | |
| Week 2 | | | | |
| Geom. Mean (Geom. Std) | 4147 (2.86) | 4/6 | 1954 (1.48) | 0/2 |
| Min; max | 2120; 33700 | | 1480; 2580 | |
| 95% CI | 1791, 9603 | | 1133, 3369 | |
| Week 8 | | | | |
| Geom. Mean (Geom. Std) | 4282 (1.94) | 5/6 | 1622 (1.55) | 0/2 |
| Min; max | 2160; 14900 | | 1190; 2210 | |
| 95% CI | 2522, 7268 | | 884, 2975 | |
| Week 10 | | | | |
| Geom. Mean (Geom. Std) | 9372 (3.55) | 5/6 | 1659 (1.28) | 0/2 |
| Min; max | 2420; 62600 | | 1390, 1980 | |
| 95% CI | 3398, 25844 | | 1173, 2347 | |
| Week 24 * | | | | |
| Geom. Mean (Geom. Std) | — | — | — | — |
| Min; max | — | | — | |
| 95% CI | — | | — | |
| Week 26 * | | | | |
| Geom. Mean (Geom. Std) | — | — | — | — |
| Min; max | — | | — | |
| 95% CI | — | | — | |
| Week 36 | | | | |
| Geom. Mean (Geom. Std) | 2709 (1.31) | 2/6 | 1627 (2.17) | 0/2 |
| Min; max | 2120; 4080 | | 942; 2810 | |
| 95% CI | 2188, 3354 | | 557, 4748 | |
| Week 48 # | | | | |
| Geom. Mean (Geom. Std) | 2097 (1.46) # | — | # | — |
| Min; max | 1140; 3220 | | | |
| 95% CI | 1551, 2835 | | | |
| Week 50 # | | | | |
| Geom. Mean (Geom. Std) | 4550 (2.83) # | — | # | — |
| Min; max | 1070; 21700 | | | |
| 95% CI | 1978, 10466 | | | |
| Week 67 # | | | | |
| Geom. Mean (Geom. Std) | 2538 (1.64) # | NA | # | NA |
| Min; max | — | | | |
| 95% CI | 1712; 3765 | | | |
| Week 74 # | | | | |
| Geom. Mean (Geom. Std) | 2150 (1.66) # | NA | # | NA |
| Min; max | — | | | |
| 95% CI | 1437, 3218 | | | |

* Due to the Covid-19 pandemic, 7/8 subjects did not receive an immunization of ACI-35.030 (300 μg dose) or placebo at week 24 and hence the data of n = 1 were not reported to avoid potential unblinding. Likewise, no blood sampling was performed at week 26.
# For sub-cohort 1.1, data at weeks 48, 50, 67 and 74 are pooled between active and placebo to avoid potential unblinding, following withdrawal of 2 subjects from the study.
NA = Data not yet available
^{♦}A responder is defined as a subject with an antibody response above the positivity threshold. A post-baseline value is considered positive if ≥ an analytical threshold × baseline. The analytical threshold is defined from samples from human donors (obtained during the validation of each assay). In particular, a responder has anti-ePHF IgG titers ≥ 2.21 × baseline.
† Baseline antibody titer value is the mean value of the titers measured at screening and visit 1 (including unscheduled visits) provided that they occur prior to the first injection.

Table 17 shows the anti-ePHF IgG titers and responder rate (ITT population) following immunization with either ACI-35.030 900 μg or placebo in sub-cohort 1.2.

TABLE 17

| | Sub-cohort 1.2 | | | |
|---|---|---|---|---|
| | ACI-35.030 900 μg | | Placebo | |
| | (n = 6) | | (n = 2) | |
| Treatment groups | Antibody titers | Responder | Antibody titers | Responder |
| (number of subjects) | (AU/mL) | rate^{♦} | (AU/mL) | rate^{♦} |
| Baseline † | | | | |
| Geom. Mean (Geom. Std) | 2858 (2.41) | — | 924 (1.18) | — |
| Min; max | 1370; 14400 | | 824; 1036 | |
| 95% CI | 1415, 5771 | | 738, 1156 | |
| Week 2 | | | | |
| Geom. Mean (Geom. Std) | 11507 (2.3) | 6/6 | 1026 (1.63) | 0/2 |
| Min; max | 4320; 38100 | | 726; 1450 | |
| 95% CI | 5909, 22411 | | 521, 2021 | |
| Week 8 | | | | |
| Geom. Mean (Geom. Std) | 9156 (2.33) | 4/6 | 914 (1.54) | 0/2 |
| Min; max | 3120; 27900 | | 673; 1240 | |
| 95% CI | 4650, 18027 | | 502, 1663 | |
| Week 10 | | | | |
| Geom. Mean (Geom. Std) | 23508 (2.14) | 6/6 | 977 (1.21) | 0/2 |
| Min; max | 5910; 49200 | | 852; 1120 | |
| 95% CI | 12768, 43284 | | 747, 1277 | |
| Week 24 | | | | |
| Geom. Mean (Geom. Std) | 11698 (1.88) | 4/6 | 831 (1.86) | 0/2 |
| Min; max | 3970; 24800 | | 535; 1290 | |
| 95% CI | 7051, 19408 | | 351, 1968 | |
| Week 26 | | | | |
| Geom. Mean (Geom. Std) | 19085 (1.54) | 6/6 | 892 (1.45) | 0/2 |
| Min; max | 10200; 34100 | | 686; 1160 | |
| 95% CI | 13499, 26983 | | 533, 1493 | |
| Week 36 | | | | |
| Geom. Mean (Geom. Std) | 18742 (1.72) | 5/6 | 1232 (1.17) | 0/2 |
| Min; max | 9440; 33400 | | 1100; 1380 | |
| 95% CI | 12132; 28954 | | 987; 1539 | |
| Week 48 | | | | |
| Geom. Mean (Geom. Std) | 11528 (1.66) | 5/6 | 1205 (1.04) | 0/2 |
| Min; max | 5730; 20000 | | 1170; 1240 | |
| 95% CI | 7695, 17271 | | 1138; 1275 | |
| Week 50 | | | | |
| Geom. Mean (Geom. Std) | 16078 (2.04) | 6/6 | 1252 (1.10) | 0/2 |
| Min; max | 7460; 38000 | | 1170; 1340 | |
| 95% CI | 9091; 28434 | | 1096; 1430 | |
| Week 67 | | | | |
| Geom. Mean (Geom. Std) | 12455 (1.91) | 5/6 | 818 (1.03) | 0/2 |
| Min; max | 5750; 32700 | | 800; 836 | |
| 95% CI | 7438; 20855 | | 783; 854 | |
| Week 74 | | | | |
| Geom. Mean (Geom. Std) | 8863 (2.12) | 3/6 | 863 (1.13) | 0/2 |
| Min; max | 2950; 25400 | | 793; 939 | |
| 95% CI | 4863; 16153 | | 731; 1018 | |

^{♦}A responder is defined as a subject with an antibody response above the positivity threshold. A post-baseline value is considered positive if ≥ an analytical threshold × baseline. The analytical threshold is defined from samples from human donors (obtained during the validation of each assay). In particular, a responder has anti-ePHF IgG titers ≥ 2.21 × baseline.
† Baseline antibody titer value is the mean value of the titers measured at screening and visit 1 (including unscheduled visits) provided that they occur prior to the first injection.

Table 18 shows the anti-ePHF IgG titers and responder rate (ITT population) following immunization with either ACI-35.030 1800 μg or placebo in sub-cohort 1.3.

TABLE 18

| | Sub-cohort 1.3 | | | |
|---|---|---|---|---|
| | ACI-35.030 1800 μg | | Placebo | |
| | (n = 6) | | (n = 2) | |
| Treatment groups | Antibody titers | Responder | Antibody titers | Responder |
| (number of subjects) | (AU/mL) | rate^{♦} | (AU/mL) | rate^{♦} |
| Baseline † | | | | |
| Geom. Mean (Geom. Std) | 1692 (2.32) | — | 893 (1.23) | — |
| Min; max | 456; 6200 | | 770; 1035 | |
| 95% CI | 862; 3319 | | 668; 1193 | |
| Week 2 | | | | |
| Geom. Mean (Geom. Std) | 4133 (2.47) | 4/6 | 955 (1.18) | 0/2 |
| Min; max | 773; 9310 | | 852; 1070 | |
| 95% CI | 2008; 8506 | | 764; 1194 | |
| Week 8 | | | | |
| Geom. Mean (Geom. Std) | 3973 (1.84) | 2/6 | 796 (1.34) | 0/2 |
| Min; max | 1900; 9470 | | 646; 980 | |
| 95% CI | 2440; 6470 | | 529; 1197 | |
| Week 10 | | | | |
| Geom. Mean (Geom. Std) | 9318 (1.98) | 5/6 | 877 (1.08) | 0/2 |
| Min; max | 4750; 28400 | | 833; 924 | |
| 95% CI | 5404; 16064 | | 793; 971 | |
| Week 24 | | | | |
| Geom. Mean (Geom. Std) | 6424 (2.88) | 3/6 | 1117 (1.11) | 0/2 |
| Min; max | 2950; 50600 | | 1040; 1200 | |
| 95% CI | 2758; 14962 | | 971; 1285 | |
| Week 26 | | | | |
| Geom. Mean (Geom. Std) | 8780 (2.38) | 4/6 | 1315 (1.04) | 0/2 |
| Min; max | 3960; 47300 | | 1280; 1350 | |
| 95% CI | 4383; 17585 | | 1248; 1385 | |
| Week 31 # | | | | |
| Geom. Mean (Geom. Std) | 9637 # | 60% # | NA | NA |
| Min; max | | | | |
| 95% CI | | | | |
| Week 36 | | | | |
| Geom. Mean (Geom. Std) | 6887 (3.14) | 3/6 | 1094 (1.05) | 0/2 |
| Min; max | 3330; 65200 | | 1060; 1130 | |
| 95% CI | 2759; 17192 | | 1028; 1165 | |
| Week 42 | | | | |
| Geom. Mean (Geom. Std) | 6025 (3.10) | 4/6 | 1048 (1.24) | 0/2 |
| Min; max | 2730; 55600 | | 900; 1220 | |
| 95% CI | 2435; 14909 | | 778; 1412 | |
| Week 48 # | | | | |
| Geom. Mean (Geom. Std) | 5887 # | 40% # | NA | NA |
| Min; max | | | | |
| 95% CI | | | | |
| Week 50 # | | | | |
| Geom. Mean (Geom. Std) | 8536 # | 80% # | NA | NA |
| Min; max | | | | |
| 95% CI | | | | |
| Week 67 | | | | |
| Geom. Mean (Geom. Std) | NA | NA | NA | NA |
| Min; max | | | | |
| 95% CI | | | | |
| Week 74 | | | | |
| Geom. Mean (Geom. Std) | NA | NA | NA | NA |
| Min; max | | | | |
| 95% CI | | | | |

NA = Data not yet available
^{♦}A responder is defined as the number of subjects with an antibody response above the positivity threshold. A post-baseline value is considered positive if ≥ an analytical threshold × baseline. The analytical threshold is defined from samples from human donors (obtained during the validation of each assay). In particular, a responder has anti-ePHF IgG titers ≥ 2.21 × baseline.
† Baseline antibody titer value is the mean value of the titers measured at screening and visit 1 (including unscheduled visits) provided that they occur prior to the first injection.
# For sub-cohort 1.3, data at weeks 31, 48 and 50 data from subjects on active treatment are pooled to avoid potential unblinding

The results of Tables 16 to 18 and FIG. 4 and FIG. 7 showed that immunization with ACI-35.030 at each of the 300 μg, 900 μg and 1800 μg doses could induce an IgG antibody response which recognizes pathological ePHF Tau derived from human AD brain. Moreover, the anti-ePHF IgG titers were boostable. The geometric mean of anti-ePHF IgG titers are increased 2 weeks after each immunization. Responder rates for IgG anti-ePHF in subjects treated with ACI-35.030 300 μg were respectively 66.7% at week 2, 83.3% at weeks 8 and 10 and 33.3% at week 36 whereas prior injection at week 24 was not performed in 7/8 subjects in the sub-cohort due to Covid-19 pandemic. In order to avoid potential study unblinding, the percentage of responders beyond week 36 in the sub-cohort are not currently reported. Responder rates for anti-ePHF IgG in subjects treated with ACI-35.030 900 μg were respectively 100% at weeks 2, 10 and 26, 66.7% at weeks 8 and 24, 83.3% at weeks 36 and 48, and 100% at week 50. It appears that the same 2 actively treated subjects were non-responders at these 2 timepoints (weeks 8 and 24) whereas they were responders at weeks 2, 10 and 26 (e.g., 2 weeks after the injections performed at weeks 0, 8 and 24 respectively). Responder rates for IgG anti-ePHF IgG in subjects treated with ACI-35.030 1800 μg at weeks 2, 8 and 10 were 66.7%, 33.3% and 83.3% respectively, then 50% at week 24, 66.7% at week 26, and 80% at week 50.

### Avidity to Pathological pTau (enriched Paired Helical Filaments—ePHF) Derived from Human AD Brain

The ability of the IgG antibody response induced by immunization with ACI-35.030 in sub-cohort 1.1, sub-cohort 1.2, and sub-cohort 1.3 for binding to ePHF derived from human AD brain was measured over time by MSD using both a low-and high-density coating of ePHF on the plate. Antibody concentration is measured on the low-density coating (only antibodies with the highest binding capacity can bind) and on the high-density coating (all antibodies can bind). The avidity index is calculated by the ratio of antibody concentrations on low/high density coating. Table 19 shows the avidity index on ePHF following immunization with ACI-35.030 at 300 μg in sub-cohort 1.1.

TABLE 19

| | Avidity index | | | | | |
|---|---|---|---|---|---|---|
| Treatment | Subject | Subject | Subject | Subject | Subject | Subject |
| groups | 1 | 2 | 3 | 4 | 5 | 6 |
| Baseline † | — | — | — | — | — | — |
| Week 2 | 0.307 | * | 0.655 | * | * | 0.51 |
| Week 8 | 0.516 | 0.462 | 0.711 | 0.545 | * | 0.743 |
| Week 10 | 0.611 | 0.484 | 0.853 | 0.593 | * | 0.733 |
| Week 24 | NA | NA | NA | NA | NA | NA |
| Week 26 | NA | NA | NA | NA | NA | NA |
| Week 36 | NA | NA | NA | NA | NA | NA |
| Week 48 | NA | NA | NA | NA | NA | NA |
| Week 50 | NA | NA | NA | NA | NA | NA |
| Week 67 | NA | NA | NA | NA | NA | NA |
| Week 74 | NA | NA | NA | NA | NA | NA |

* measurement not valid

Table 20 shows the avidity index on ePHF following immunization with ACI-35.030 at 900 μg in sub-cohort 1.2.

TABLE 20

| | Avidity index | | | | | |
|---|---|---|---|---|---|---|
| Treatment | Subject | Subject | Subject | Subject | Subject | Subject |
| groups | 1 | 2 | 3 | 4 | 5 | 6 |
| Baseline † | — | — | — | — | — | — |
| Week 2 | 0.508 | 0.383 | 0.571 | 0.484 | 0.682 | 0.788 |
| Week 8 | 0.554 | 0.487 | 0.639 | 0.464 | * | * |
| Week 10 | * | 0.678 | 0.676 | * | 0.707 | 0.822 |
| Week 24 | * | 0.743 | 0.656 | * | * | * |
| Week 26 | * | 0.754 | 0.433 | * | * | 0.777 |
| Week 36 | * | 0.73 | * | * | * | * |
| Week 48 | * | 0.732 | * | * | * | * |
| Week 50 | * | 0.714 | * | * | * | 1.14 |
| Week 67 | * | 0.748 | * | * | * | * |
| Week 74 | * | 0.87 | * | * | * | * |

* measurement not valid

Table 21 shows the avidity index on ePHF following immunization with ACI-35.030 at 1800 μg in sub-cohort 1.3.

TABLE 21

| | Avidity index | | | | | |
|---|---|---|---|---|---|---|
| Treatment | Subject | Subject | Subject | Subject | Subject | Subject |
| groups | 1 | 2 | 3 | 4 | 5 | 6 |
| Baseline † | — | — | — | — | — | — |
| Week 2 | * | 0.617 | 0.559 | 0.419 | 0.383 | * |
| Week 8 | 0.550 | * | * | * | 0.408 | * |
| Week 10 | 0.702 | 0.786 | 0.714 | 0.557 | 0.338 | * |
| Week 24 | 0.598 | * | 0.458 | * | * | * |
| Week 26 | 0.559 | * | * | 0.495 | * | * |
| Week 36 | 0.593 | * | * | 0.474 | * | * |
| Week 42 | 0.736 | * | 0.752 | 0.53 | * | * |
| Week 48 | NA | NA | NA | NA | NA | NA |
| Week 50 | NA | NA | NA | NA | NA | NA |
| Week 67 | NA | NA | NA | NA | NA | NA |
| Week 74 | NA | NA | NA | NA | NA | NA |

* measurement not valid

The results of Tables 19, 20, and 21 showed that, in most patients, immunization with ACI-35.030 at each of 300 μg, 900 and 1800 μg doses induced an IgG immune response which shows an increase in binding avidity between week 2 and week 10.

To-date, the preliminary results showed that, for example, ACI-35.030 induced a high, specific and sustained antibody response oriented toward Tau-pathological species (phospho-Tau and ePHF), with an apparent dose-response between the low-and mid-dose with evidence of immunoglobulin class switch from IgM to IgG. Individual responder rates were high and consistent, especially for anti-pTau and ePHF antibodies. The administration of ACI-35.030 did not appear to have caused any particular safety concerns related to the study vaccine as of the date of the data analyses providing support for ACI-35.030's favorable safety and tolerability profile, and ACI-35.030 was able to induce a lasting antibody response above the baseline values in immunized patients. Over time, the data demonstrates that the IgG response matures towards a stronger preference for binding ePHF, the more pathologic species, while concomitantly lowering antibody titers towards the non-pathological, non-phosphorylated Tau. The preliminary results support the further development of this vaccine as an effective AD disease-modifying treatment as well as an approach for a potential prevention of AD.

### Example 2 Vaccination With ACI-35.030 Induces Relatively Homogeneous Antibody Response With a Broad Epitope Coverage

To further profile the antibody response for breadth and selectivity towards pathological pTau, epitope mapping was performed on the human subjects' sera. A study was performed to determine the epitope recognition profile of antibodies induced by the liposome vaccine in human subjects. Seven Alzheimer's Disease (AD) patients were immunized intramuscularly at week 0, 8, 24, and 48 with 900 μg of acetate tetrapalmitoylated phosphorylated Tau peptide T3 (SEQ ID NO:28) per dose of liposome vaccine or placebo (sub-cohort 1.2) in a phase 1/2 clinical study. The epitope recognition profile of antibodies was determined by epitope mapping ELISA before the first immunization (V1, week 0) and after the third immunization (V6, week 26) using a library of N-terminally biotinylated 8-mer peptides, shifted by one amino acid and covering the entire sequence of phospho-Tau peptide T3.30 (SEQ ID NO: 45) as well as the corresponding sequence of non-phosphorylated Tau peptide T3.56 (SEQ ID NO: 46). In addition, binding of antibodies to a full-length phospho-Tau peptide T3.30 (and Tau peptide T3.56 as well as another N-terminally biotinylated phospho-Tau peptide T3.85 (SEQ ID NO: 47) and the corresponding non-phosphorylated Tau peptide T3.86 (SEQ ID NO: 48) (with an additional C-terminal amino acid) was determined.

Data are expressed as pre-treatment-subtracted optical density (O.D.) values obtained before the initial immunization (V1, week 0) subtracted from O.D. obtained after the third immunization (V6, week 26) for each peptide and each patient. Negative values after subtraction were set to 0.000.

Tables 22 and 23 show the epitope recognition profile of antibodies induced by vaccination with ACI-35.030, as determined by epitope mapping ELISA on short 8-mer overlapping peptides, covering phospho-peptides T3.30 and T3.85 and non-phospho-peptides T3.56 and T3.86.

TABLE 22

| Phospho-Tau | Patient | | | | | | |
|---|---|---|---|---|---|---|---|
| peptide | #1 | #2 | #3 | #4 | #5 | #6 | #7 |
| pTau393-400 | 0.000 | 0.000 | 2.788 | 3.131 | 2.582 | 3.110 | 1.208 |
| pTau394-401 | 0.003 | 0.000 | 1.885 | 2.163 | 1.491 | 1.721 | 0.553 |
| pTau395-402 | 0.000 | 0.000 | 1.769 | 0.926 | 1.292 | 0.495 | 0.319 |
| pTau396-403 | 0.000 | 0.000 | 0.117 | 0.272 | 0.734 | 0.284 | 0.150 |
| pTau397-404 | 0.002 | 0.000 | 0.078 | 0.113 | 0.459 | 0.087 | 0.082 |
| pTau398-405 | 0.000 | 0.000 | 0.119 | 0.155 | 0.342 | 0.660 | 0.173 |
| pTau399-406 | 0.020 | 0.000 | 0.063 | 0.157 | 0.163 | 0.627 | 0.098 |
| pTau400-407 | 0.012 | 0.000 | 0.277 | 0.294 | 0.060 | 0.616 | 0.118 |
| pTau401-408 | 0.000 | 0.000 | 0.239 | 1.262 | 0.381 | 0.253 | 0.147 |
| pTau393-408 | 0.000 | 0.000 | 3.617 | 3.558 | 2.529 | 3.548 | 3.024 |
| pTau393-409 | 0.001 | 0.000 | 3.546 | 3.404 | 3.411 | 3.509 | 2.207 |

TABLE 23

| Tau | Patient | | | | | | |
|---|---|---|---|---|---|---|---|
| peptide | #1 | #2 | #3 | #4 | #5 | #6 | #7 |
| Tau393-400 | 0.023 | 0.000 | 1.688 | 0.165 | 0.000 | 0.105 | 0.173 |
| Tau394-401 | 0.008 | 0.006 | 1.155 | 0.071 | 0.000 | 0.060 | 0.087 |
| Tau395-402 | 0.007 | 0.018 | 1.694 | 0.077 | 0.020 | 0.070 | 0.102 |
| Tau396-403 | 0.005 | 0.000 | 0.050 | 0.113 | 0.063 | 0.097 | 0.188 |
| Tau397-404 | 0.013 | 0.000 | 0.016 | 0.053 | 0.047 | 0.044 | 0.076 |
| Tau398-405 | 0.014 | 0.001 | 0.005 | 0.095 | 0.001 | 0.046 | 0.070 |
| Tau399-406 | 0.020 | 0.000 | 0.011 | 0.116 | 0.000 | 0.034 | 0.062 |
| Tau400-407 | 0.001 | 0.000 | 0.298 | 0.350 | 0.031 | 0.110 | 0.083 |
| Tau401-408 | 0.008 | 0.000 | 0.621 | 1.768 | 0.439 | 0.175 | 0.230 |
| Tau393-408 | 0.010 | 0.000 | 3.323 | 1.197 | 0.714 | 0.251 | 0.585 |
| Tau393-409 | 0.009 | 0.000 | 3.429 | 0.244 | 0.264 | 0.129 | 0.452 |

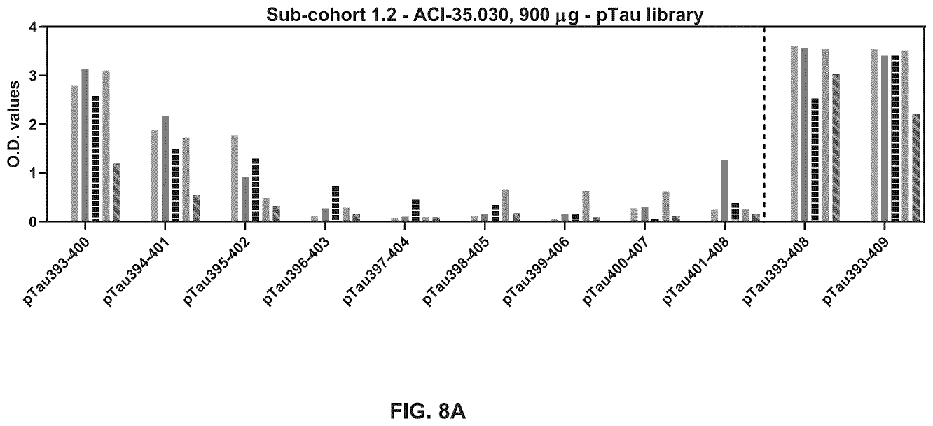

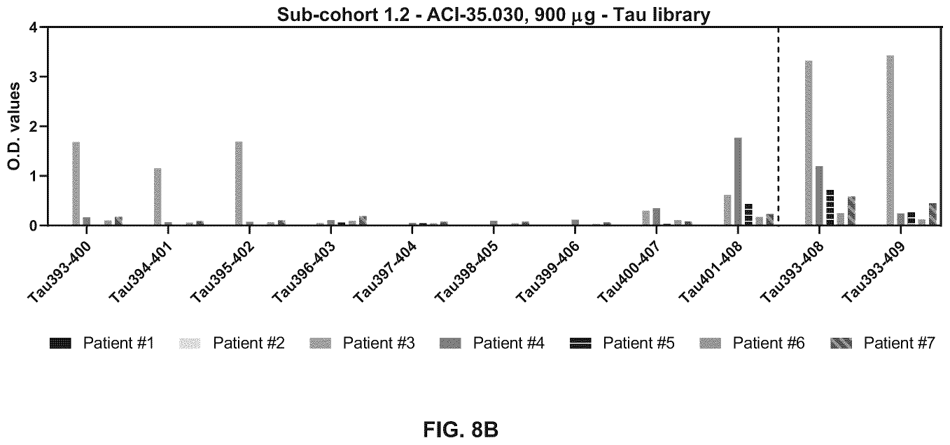

Table 22 and FIG. 8A shows that two AD patients essentially did not produce any IgG antibodies after three immunizations at week 26 against the sequences of or within phospho-Tau peptides T3.30 and T3.85 (patients #1 and #2), whereas the other five AD patients generated IgG antibodies against the sequences of or within phospho-Tau peptides T3.30 and T3.85 with overall similar binding to the sequences of phospho-Tau peptides T3.30 and T3.85. O.D. values obtained on 8-mer peptides indicate that IgG antibodies induced after three immunizations bound mostly to the N-terminal part of the sequence of phospho-Tau peptides T3.30 (SEQ ID NO: 45) and T3.85 (SEQ ID NO: 47), including the phosphorylated serine at position 396. Overall lower binding was observed to the C-terminal part of the sequence of phospho-Tau peptides T3.30 (SEQ ID NO: 45) and T3.85 (SEQ ID NO: 47), including the phosphorylated serine at position 404.

Table 23 and FIG. 8B shows that two AD patients essentially did not produce any IgG antibodies at week 26 against the sequence of non-phosphorylated Tau peptides T3.56 and T3.86 (patients #1 and #2). Four AD patients generated IgG antibodies with minor recognition of the sequences of or within the non-phosphorylated Tau peptides T3.56 and T3.86, and the binding appeared to be linked to binding of the C-terminal 8-mer peptide (tau401-408). One AD patient (patient #3) produced IgG antibodies against the sequences of or within the non-phosphorylated Tau peptides T3.56 and T3.86, and the binding appears to be mostly linked to the N-terminal part of the sequence.

For ACI-35.030, the IgG response of the subjects was relatively homogenous displaying a broad epitope coverage as binding occurred across the pTau sequences tested and importantly, without substantial specificity for terminal end of the peptide sequence or substantial binding to non-phosphorylated sequences.

SEQUENCE LISTING

SEQ ID NO: 1 - phospho-Tau peptide (7.1)
GDRSGYS[pS]PG[pS]PG[pT]PGSRSRT
SEQ ID NO: 2 - phospho-Tau peptide (T3.5)
VYK[pS]PVVSGDT[pS]PRHL
SEQ ID NO: 3 - phospho-Tau peptide (22.1)
SSTGSIDMVD[pS]PQLA[pT]LA
SEQ ID NO: 4 - Tau peptide (T3.6)
VYKSPVVSGDTSPRHL
SEQ ID NO: 5 - phospho-Tau peptide
RENAKAKTDHGAEIVYK[pS]PVVSGDT[pS]PRHL
SEQ ID NO: 6 - phospho-Tau peptide
RQEFEVMEDHAGT[pY]GL
SEQ ID NO: 7 - phospho-Tau peptide
PGSRSR[pT]P[pS]LPTPPTR
SEQ ID NO: 8 - phospho-Tau peptide
GYSSPG[pS]PG[pT]PGSRSR
SEQ ID NO: 9 - phospho-Tau peptide
GDT[pS]PRHL[pS]NVSSTGSID
SEQ ID NO: 10 - phospho-Tau peptide
PG[pS]PG[pT]PGSRSR[pT]P[pS]LP
SEQ ID NO: 11 - phospho-Tau peptide
HL[pS]NVSSTGSID
SEQ ID NO: 12 - phospho-Tau peptide
VSGDT[pS]PRHL
SEQ ID NO: 13 - T50 T cell epitope
AKFVAAWTLKAAAVVRQYIKANSKFIGITELVVRFNNFTVSFWLRVPKVS
ASHLE-NH₂
SEQ ID NO: 14 - T46 T cell epitope
AKFVAAWTLKAAAGSQYIKANSKFIGITELGSFNNFTVSFWLRVPKVSAS
HLEK(Pal)K(Pal)-NH₂
SEQ ID NO: 15 - T48 helper T cell epitope
AKFVAAWTLKAAAGSQYIKANSKFIGITELGSFNNFTVSFWLRVPKVSAS
HLEGSLINSTKIYSYFPSVISKVNQ-NH₂
SEQ ID NO: 16 - T51 helper T cell epitope
AKFVAAWTLKAAARRQYIKANSKFIGITELRRFNNFTVSFWLRVPKVSAS
HLE-NH₂
SEQ ID NO: 17 - T52 helper T cell epitope
AKFVAAWTLKAAARKQYIKANSKFIGITELRKFNNFTVSFWLRVPKVSAS
HLE-NH₂
SEQ ID NO: 18 - CpG 2006 (also known as CpG 7909)
5′-tcgtcgttttgtcgttttgtcgtt-3′
wherein lower case means phosphorothioate (ps)
internucleotide linkages
SEQ ID NO: 19 - CpG 1018
5′-tgactgtgaacgttcgagatga-3′
wherein lower case means phosphorothioate
internucleotide linkages
SEQ ID NO: 20 - CpG2395
5′-tcgtcgttttcggcgcgcgccg-3′
wherein lower case means phosphorothioate
internucleotide linkages
SEQ ID NO: 21 - CpG2216
5′-ggGGGACGATCGTCgggggg-3′
wherein lower case means phosphorothioate
internucleotide linkages and capital letters
means phosphodiester (po) linkages
SEQ ID NO: 22 - CpG2336
5′-gggGACGACGTCGTGgggggg-3′,
wherein lower case means phosphorothioate
internucleotide linkages and capital letters
means phosphodiester linkages
SEQ ID NO: 23 - Pan DR epitope (PADRE) peptide
AKFVAAWTLKAAA
SEQ ID NO: 24 - P2
QYIKANSKFIGITEL
SEQ ID NO: 25 - P30
FNNFTVSFWLRVPKVSASHLE
SEQ ID NO: 26 - TT₅₈₆₋₆₀₅
LINSTKIYSYFPSVISKVNQ
SEQ ID NO: 27 - palmitoylated phospho-Tau peptide
(palmitoylated 7.1)
K(pal)K(pal)GDRSGYS[pS]PG[pS]PG[pT]PGSRSRTK(pal)
K(pal)
SEQ ID NO: 28 - palmitoylated phospho-Tau peptide
(T3, palmitoylated T3.5)
K(pal)K(pal)VYK[pS]PVVSGDT[pS]PRHLK(pal)K(pal)
SEQ ID NO: 29 - palmitoylated phospho-Tau peptide
(palmitoylated 22.1)
K(pal)K(pal)SSTGSIDMVD[pS]PQLA[pT]LAK(pal)K(pal)
SEQ ID NO: 30 - palmitoylated Tau peptide
K(pal)K(pal)VYKSPVVSGDTSPRHLK(pal)K(pal)
SEQ ID NO: 31 - palmitoylated phospho-Tau peptide
K(pal)K(pal)RENAKAKTDHGAEIVYK[pS]PVVSGDT[pS]PRHL
K(pal)K(pal)
SEQ ID NO: 32 - palmitoylated phospho-Tau peptide
K(pal)K(pal)RQEFEVMEDHAGT[pY]GLK(pal)K(pal)
SEQ ID NO: 33 - palmitoylated phospho-Tau peptide
K(pal)K(pal)PGSRSR[pT]P[pS]LPTPPTRK(pal)K(pal)
SEQ ID NO: 34 - palmitoylated phospho-Tau peptide
K(pal)K(pal)GYSSPG[pS]PG[pT]PGSRSRK(pal)K(pal)
SEQ ID NO: 35 - palmitoylated phospho-Tau peptide
K(pal)K(pal)GDT[pS]PRHL[pS]NVSSTGSIDK(pal)K(pal)
SEQ ID NO: 36 - palmitoylated phospho-Tau peptide
K(pal)K(pal)PG[pS]PG[pT]PGSRSR[pT]P[pS]LPK(pal)
K(pal)
SEQ ID NO: 37 - palmitoylated phospho-Tau peptide
K(pal)K(pal)HL[pS]NVSSTGSIDK(pal)K(pal)
SEQ ID NO: 38 - palmitoylated phospho-Tau peptide
K(pal)K(pal)VSGDT[pS]PRHLK(pal)K(pal)
SEQ ID NO: 39 - T50 without the C-terminal amide
AKFVAAWTLKAAAVVRQYIKANSKFIGITELVVRFNNFTVSFWLRVPKVS
ASHLE
SEQ ID NO: 40 - T46 without the -Lys(Pal)-
Lys(Pal)-NH₂ at the C-terminal
AKFVAAWTLKAAAGSQYIKANSKFIGITELGSFNNFTVSFWLRVPKVSAS
HLE
SEQ ID NO: 41 - T48 without the C-terminal amide
AKFVAAWTLKAAAGSQYIKANSKFIGITELGSFNNFTVSFWLRVPKVSAS
HLEGSLINSTKIYSYFPSVISKVNQ
SEQ ID NO: 42 - T51 without the C-terminal amide
AKFVAAWTLKAAARRQYIKANSKFIGITELRRFNNFTVSFWLRVPKVSAS
HLE
SEQ ID NO: 43 - T52 without the C-terminal amide
AKFVAAWTLKAAARKQYIKANSKFIGITELRKFNNFTVSFWLRVPKVSAS
HLE
SEQ ID NO: 44 - T57
AKFVAAWTLKAAAVVRQYIKANSKFIGITELVVRFNNFTVSFWLRVPKVS
ASHLE-K(Pal)K(Pal)-NH2
SEQ ID NO: 45 - biotinylated phosphorylated tau
peptide (T3.30)
Biotin-LC linker(Ahx)-GVYK[pS]PVVSGDT[pS]PRHL-NH2
SEQ ID NO: 46 - biotinylated non-phosphorylated
tau peptide (T3.56)
Biotin-LC linker(Ahx)-GVYKSPVVSGDTSPRHL-NH2
SEQ ID NO: 47 - biotinylated phosphorylated tau
peptide (T3.85)
Biotin-LC linker(Ahx)-VYK[pS]PVVSGDT[pS]PRHLS-NH2
SEQ ID NO: 48 - biotinylated non-phosphorylated
tau peptide (T3.86)
Biotin-LC linker(Ahx)-VYKSPVVSGDTSPRHLS-NH2

It is claimed:

1. A method of inducing at least 20 weeks of an immune response against a phosphorylated Tau protein (pTau) in a human subject in need thereof, comprising:
i. intramuscularly administering to the subject a primer vaccine comprising an effective amount of a liposome; and
ii. intramuscularly administering to the subject a first booster vaccine comprising the effective amount of the liposome 6-10 weeks after the administration of the primer vaccine, wherein:
(a) the immune response lasts at least 20 weeks after the administration of the primer vaccine;
(b) the liposome comprises:
(1) a Tau phosphopeptide consisting of the amino acid sequence of SEQ ID NO: 28, and the Tau phosphopeptide is presented on the surface of the liposome;
(2) a toll-like receptor 4 agonist comprising monophosphoryl lipid A;
(3) a helper T-cell epitope having an amino acid sequence selected from the group consisting of SEQ ID NOs:23, 24, 25, and 26; and
(4) a CpG oligonucleotide having a nucleotide sequence selected from the group consisting of SEQ ID NO:18 to SEQ ID NO:22; and
(c) the effective amount of the liposome comprises the Tau phosphopeptide at an amount of 300 μg to 1800 μg per dose, and
wherein the immune response is boosted after the administration of the first booster vaccine as measured at least 2 weeks after the administration of the first booster vaccine.

2. The method of claim 1, wherein the effective amount of the liposome comprises:
(1) the Tau phosphopeptide at the amount of 300 μg to 1800 μg per dose;
(2) the toll-like receptor 4 agonist at an amount of 100 μg to 585 μg per dose;
(3) the helper T-cell epitope at an amount of 75 μg to 550 μg per dose; and
(4) the CpG oligonucleotide at an amount of 150 μg to 900 μg per dose.

3. The method of claim 1, wherein the effective amount of the liposome comprises 300 μg, 900 μg or 1800 μg per dose of the Tau phosphopeptide.

4. The method of claim 1, wherein the CpG oligonucleotide has one or more phosphorothioate internucleotide linkages, and the CpG oligonucleotide is covalently linked to at least one lipophilic group, optionally via a PEG linker.

5. The method of claim 1, wherein the immune response comprises an IgG immune response that preferentially recognizes the pTau over non-phosphorylated Tau protein with a ratio of the anti-pTau IgG titer to the anti-Tau IgG titer of at least 5.

6. The method of claim 1, wherein the immune response further comprises a class switch of a specific IgM antibody response to a specific IgG antibody response directed against the pTau, and/or an IgG immune response against an enriched Paired Helical Filament (ePHF) having an anti-ePHF IgG titer at least 2 times higher than that of a placebo control, and the anti-ePHF IgG has an increased binding avidity to the pathological ePHF Tau with an avidity index of at least 0.3.

7. The method of claim 1, wherein the subject is in need of a treatment of Alzheimer's Disease.

8. The method of claim 1, wherein the helper T cell epitope comprises an amino acid sequence selected from the group consisting of SEQ ID NOs: 13, 14, 15, 16, 17, 39, 40, 41, 42, 43 and 44.

9. A method of inducing at least 36 weeks of an immune response against a phosphorylated Tau protein (pTau) in a human subject in need thereof, comprising:
i. intramuscularly administering to the subject a primer vaccine comprising an effective amount of a liposome;
ii. intramuscularly administering to the subject a first booster vaccine comprising the effective amount of the liposome 6-10 weeks after the administration of the primer vaccine; and
iii. intramuscularly administering to the subject a second booster vaccine comprising the effective amount of the liposome 22-26 weeks after the administration of the primer vaccine, wherein:
(a) the immune response lasts at least 36 weeks after the administration of the primer vaccine;
(b) the liposome comprises:
(1) a Tau phosphopeptide consisting of the amino acid sequence of SEQ ID NO: 28, and the Tau phosphopeptide is presented on the surface of the liposome;
(2) a toll-like receptor 4 agonist comprising monophosphoryl lipid A;
(3) a helper T-cell epitope having an amino acid sequence selected from the group consisting of SEQ ID NOs:23, 24, 25, and 26; and
(4) a CpG oligonucleotide having a nucleotide sequence selected from the group consisting of SEQ ID NO:18 to SEQ ID NO:22; and
(c) the effective amount of the liposome comprises the Tau phosphopeptide at an amount of 300 μg to 1800 μg per dose, and
wherein the immune response is boosted after the administration of each of the first booster vaccine and the second booster vaccine as measured at least 2 weeks after the administration of each of the first booster vaccine and the second booster vaccine, respectively.

10. The method of claim 9, wherein the effective amount of the liposome comprises:
(1) the Tau phosphopeptide at the amount of 300 μg to 1800 μg per dose;
(2) the toll-like receptor 4 agonist at an amount of 100 μg to 585 μg per dose;
(3) the helper T-cell epitope at an amount of 75 μg to 550 μg per dose; and
(4) the CpG oligonucleotide at an amount of 150 μg to 900 μg per dose.

11. The method of claim 9, wherein the effective amount of the liposome comprises 300 μg, 900 μg or 1800 μg per dose of the Tau phosphopeptide.

12. The method of claim 9, wherein the CpG oligonucleotide has one or more phosphorothioate internucleotide linkages, and the CpG oligonucleotide is covalently linked to at least one lipophilic group, optionally via a PEG linker.

13. The method of claim 9, wherein the immune response comprises an IgG immune response that preferentially recognizes the pTau over non-phosphorylated Tau protein with a ratio of the anti-pTau IgG titer to the anti-Tau IgG titer of at least 5.

14. The method of claim 9, wherein the immune response further comprises a class switch of a specific IgM antibody response to a specific IgG antibody response directed against the pTau, and/or an IgG immune response against an enriched Paired Helical Filament (ePHF) having an anti-ePHF IgG titer at least 2 times higher than that of a placebo control, and the anti-ePHF IgG has an increased binding avidity to the pathological ePHF Tau with an avidity index of at least 0.3.

15. The method of claim 9, wherein the subject is in need of a treatment of Alzheimer's Disease.

16. A method of inducing at least 60 weeks of an immune response against a phosphorylated Tau protein (pTau) in a human subject in need thereof, comprising:
i. intramuscularly administering to the subject a primer vaccine comprising an effective amount of a liposome;
ii. intramuscularly administering to the subject a first booster vaccine comprising the effective amount of the liposome 6-10 weeks after the administration of the primer vaccine;
iii. intramuscularly administering to the subject a second booster vaccine comprising the effective amount of the liposome 22-26 weeks after the administration of the primer vaccine; and
iv. intramuscularly administering to the subject a third booster vaccine comprising the effective amount of the liposome 45-50 weeks after the administration of the primer vaccine, wherein:
(a) the immune response lasts at least 60 weeks after the administration of the primer vaccine;
(b) the liposome comprises:
(1) a Tau phosphopeptide consisting of the amino acid sequence of SEQ ID NO: 28, and the Tau phosphopeptide is presented on the surface of the liposome;
(2) a toll-like receptor 4 agonist comprising monophosphoryl lipid A;
(3) a helper T-cell epitope having an amino acid sequence selected from the group consisting of SEQ ID NOs:23, 24, 25, and 26; and
(4) a CpG oligonucleotide having a nucleotide sequence selected from the group consisting of SEQ ID NO:18 to SEQ ID NO:22; and
(c) the effective amount of the liposome comprises the Tau phosphopeptide at an amount of 300 μg to 1800 μg per dose, and
wherein the immune response is boosted after the administration of each of the first booster vaccine, the second booster vaccine and the third booster vaccine as measured at least 2 weeks after the administration of each of the first booster vaccine, the second booster vaccine and the third booster vaccine, respectively.

17. The method of claim 16, wherein the effective amount of the liposome comprises:
(1) the Tau phosphopeptide at the amount of 300 μg to 1800 μg per dose;
(2) the toll-like receptor 4 agonist at an amount of 100 μg to 585 μg per dose;
(3) the helper T-cell epitope at an amount of 75 μg to 550 μg per dose; and
(4) the CpG oligonucleotide at an amount of 150 μg to 900 μg per dose.

18. The method of claim 16, wherein the effective amount of the liposome comprises 300 μg, 900 μg or 1800 μg per dose of the Tau phosphopeptide.

19. The method of claim 16, wherein the CpG oligonucleotide has one or more phosphorothioate internucleotide linkages, and the CpG oligonucleotide is covalently linked to at least one lipophilic group, optionally via a PEG linker.

20. The method of claim 16, wherein the immune response comprises an IgG immune response that preferentially recognizes the pTau over non-phosphorylated Tau protein with a ratio of the anti-pTau IgG titer to the anti-Tau IgG titer of at least 5.

21. The method of claim 16, wherein the immune response further comprises a class switch of a specific IgM antibody response to a specific IgG antibody response directed against the pTau, and/or an IgG immune response against an enriched Paired Helical Filament (ePHF) having an anti-ePHF IgG titer at least 2 times higher than that of a placebo control, and the anti-ePHF IgG has an increased binding avidity to the pathological ePHF Tau with an avidity index of at least 0.3.

22. The method of claim 16, wherein the subject is in need of a treatment of Alzheimer's Disease.

23. A method of inducing at least 20 weeks of an immune response against a phosphorylated Tau protein (pTau) in a human subject in need thereof, comprising:
i. intramuscularly administering to the subject a primer vaccine comprising an effective amount of a liposome; and
ii. intramuscularly administering to the subject a first booster vaccine comprising an effective amount of the liposome 6-10 weeks after the administration of the primer vaccine, wherein:
(a) the immune response lasts at least 20 weeks after the administration of the primer vaccine;
(b) the liposome comprises:
(1) a Tau phosphopeptide consisting of the amino acid sequence of SEQ ID NO: 28;
(2) a toll-like receptor 4 agonist comprising monophosphoryl lipid A;
(3) a helper T-cell epitope having an amino acid sequence selected from the group consisting of SEQ ID NOs:13, 14 or 15; and
(4) a CpG oligonucleotide having the nucleotide sequence consisting of SEQ ID NO:18,
(c) the effective amount of the liposome comprises the Tau phosphopeptide at an amount of 900 μg per dose, and
wherein the immune response is boosted after the administration of the first booster vaccine as measured at least 2 weeks after the administration of the first booster vaccine.

24. The method of claim 23, wherein the effective amount of the liposome comprises:
(1) the Tau phosphopeptide at the amount of 900 μg per dose;
(2) the toll-like receptor 4 agonist at an amount of 100 μg to 585 μg per dose;
(3) the helper T-cell epitope at an amount of 75 μg to 550 μg per dose; and
(4) the CpG oligonucleotide at an amount of 150 μg to 900 μg per dose.

25. The method of claim 23, wherein the CpG oligonucleotide has one or more phosphorothioate internucleotide linkages, and the CpG oligonucleotide is covalently linked to at least one lipophilic group, optionally via a PEG linker.

26. The method of claim 23, further comprising intramuscularly administering to the subject a second booster vaccine comprising the effective amount of the liposome 22-26 weeks after the administration of the primer vaccine, wherein the immune response is boosted after the administration of the second booster vaccine as measured at least 2 weeks after the administration of the second booster vaccine and the immune response lasts at least 36 weeks after the administration of the primer vaccine.

27. The method of claim 26, further comprising intramuscularly administering to the subject a third booster vaccine comprising the effective amount of the liposome 45-50 weeks after the administration of the primer vaccine, wherein the immune response is boosted after the administration of the third booster vaccine as measured at least 2 weeks after the administration of the third booster vaccine and the immune response lasts at least 60 weeks after the administration of the primer vaccine.

28. The method of claim 23, wherein the immune response comprises an IgG immune response that preferentially recognizes the pTau over non-phosphorylated Tau protein with a ratio of the anti-pTau IgG titer to the anti-Tau IgG titer of at least 5.

29. The method of claim 23, wherein the immune response further comprises a class switch of a specific IgM antibody response to a specific IgG antibody response directed against the pTau, and/or an IgG immune response against an enriched Paired Helical Filament (ePHF) having an anti-ePHF IgG titer at least 2 times higher than that of a placebo control, and the anti-ePHF IgG has an increased binding avidity to the pathological ePHF Tau with an avidity index of at least 0.3.

30. The method of claim 23, wherein the subject is in need of a treatment of Alzheimer's Disease.
